(19) United States
(12) Patent Application Publication
(10) Pub. No.: US 2021/0059614 A1
(43) Pub. Date: Mar. 4, 2021

(54) SARCOPENIA EVALUATION METHOD, SARCOPENIA EVALUATION DEVICE, AND NON-TRANSITORY COMPUTER-READABLE RECORDING MEDIUM IN WHICH SARCOPENIA EVALUATION PROGRAM IS RECORDED

(71) Applicant: Panasonic Intellectual Property Corporation of America, Torrance, CA (US)

(72) Inventors: Takahiro HIYAMA, Tokyo (JP); Yoshikuni SATO, Tokyo (JP); Takahiro AIHARA, Osaka (JP); Kengo WADA, Osaka (JP); Taichi HAMATSUKA, Osaka (JP); Yoshihiro MATSUMURA, Osaka (JP)

(21) Appl. No.: 16/991,551

(22) Filed: Aug. 12, 2020

Related U.S. Application Data
(60) Provisional application No. 62/893,294, filed on Aug. 29, 2019.

(30) Foreign Application Priority Data
Feb. 14, 2020 (JP) 2020-023431

(51) Int. Cl. A61B 5/00; A61B 5/11; G16H 20/30; G16H 50/50

(52) U.S. Cl. CPC A61B 5/7282; A61B 5/112; A61B 5/1121; A61B 5/1128; A61B 2503/08; A61B 5/4595; A61B 5/4585; G16H 20/30; G16H 50/50; A61B 5/0077

(57) ABSTRACT

A sarcopenia evaluation method in a sarcopenia evaluation device that evaluates sarcopenia based on a walking motion of a subject includes: acquiring walking data related to walking of the subject; detecting, from the walking data, at least one walking parameter of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of an ankle joint of the one foot in the stance phase, and an angle of the ankle joint of the one foot in the swing phase; and determining whether or not the subject has sarcopenia using at least one walking parameter.

### FIELD OF THE INVENTION

The present disclosure relates to a technology for evaluating sarcopenia based on walking motion of a subject.

### BACKGROUND ART

In recent years, in order to grasp the health condition of the elderly, a technology for easily estimating a physical function has been developed. In particular, as for the elderly, muscle mass decreases and fat mass increases, with age. In general, a condition showing a decrease in skeletal muscle mass with aging is called sarcopenia. Sarcopenia is said to be closely associated with falls, fractures, bedridden, and weakness. Therefore, it is necessary to find elderly people with sarcopenia early and take countermeasures.

Conventionally, technologies have been proposed for evaluating cognitive functions or motor functions based on parameters measured from daily walking.

For example, Japanese Patent Application Laid-Open No. 2013-255786 discloses a method for evaluating the likelihood of a senile disorder (senile disorder risk) based on walking parameters measured by walking.

In Japanese Patent Application Laid-Open No. 2018-114319, for example, acceleration sensor attached to the waist of a subject measures a longitudinal acceleration, a lateral acceleration, and a vertical acceleration during the movement of the subject, and the movement ability is evaluated based on temporal changes in the longitudinal acceleration, the lateral acceleration, and the vertical acceleration.

However, with the above-mentioned conventional technologies, it is difficult to easily and highly accurately evaluate sarcopenia, and further improvement has been required.

### SUMMARY OF THE INVENTION

The present disclosure has been made to solve the above problems, and an object of the present disclosure is to provide a technology capable of easily and highly accurately evaluating sarcopenia.

A sarcopenia evaluation method according to an aspect of the present disclosure is a sarcopenia evaluation method in a sarcopenia evaluation device that evaluates sarcopenia based on the walking motion of a subject, the sarcopenia evaluation method including: acquiring walking data related to walking of the subject; detecting, from the walking data, at least one of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of an ankle joint of the one foot in the stance phase, and an angle of the ankle joint of the one foot in the swing phase; and determining whether or not the subject has sarcopenia using at least one of the angle of the knee joint in the stance phase, the angle of the knee joint in the swing phase, the vertical displacement of the toe in the stance phase, the vertical displacement of the toe in the swing phase, the angle of the ankle joint in the stance phase, and the angle of the ankle joint in the swing phase.

### BRIEF DESCRIPTION OF THE DRAWINGS

FIG. 1 is a block diagram showing a configuration of a sarcopenia evaluation system in an embodiment of the present disclosure;

FIG. 2 is a view for explaining processing of extracting skeleton data from two-dimensional image data in the present embodiment;

FIG. 3 is a view for explaining a walking cycle in the present embodiment;

FIG. 4 is a flowchart for explaining sarcopenia evaluation processing using a walking motion of a subject in the present embodiment;

FIG. 5 is a flowchart for explaining the sarcopenia determination processing in step S4 of FIG. 4;

FIG. 6 is a flowchart for explaining another example of the sarcopenia determination processing in step S4 of FIG. 4;

FIG. 7 is a view showing a change in the angle of the knee joint of one leg in one walking cycle in the present embodiment;

FIG. 8 is a view showing an ROC curve obtained as a result of determining a healthy subject and a sarcopenia subject using a prediction model in the present embodiment;

FIG. 9 is a view showing an ROC curve obtained as a result of determining a healthy subject and a pre-sarcopenia subject using the prediction model in the present embodiment;

FIG. 10 is a view showing an ROC curve obtained as a result of determining a healthy subject and a sarcopenia subject using a prediction model in a first modification of the present embodiment;

FIG. 11 is a view showing an ROC curve obtained as a result of determining a healthy subject and a pre-sarcopenia subject using the prediction model in the first modification of the present embodiment;

FIG. 12 is a view showing an ROC curve obtained as a result of determining a healthy subject and a sarcopenia subject using a prediction model in a second modification of the present embodiment;

FIG. 13 is a view showing an ROC curve obtained as a result of determining a healthy subject and a pre-sarcopenia subject using the prediction model in the second modification of the present embodiment;

FIG. 14 is a view showing an average of mean values of time series data of the angle of the knee joint of one leg of sarcopenia subjects, an average of mean values of time series data of the angle of the knee joint of one leg of pre-sarcopenia subjects, and an average of mean values of time series data of the angle of the knee joint of one leg of healthy subjects in the second modification of the present embodiment;

FIG. 15 is a view showing a vertical displacement of the toe of one foot in one walking cycle in a third modification of the present embodiment;

FIG. 16 is a view showing an ROC curve obtained as a result of determining a healthy subject and a sarcopenia subject using a prediction model in the third modification of the present embodiment;

FIG. 17 is a view showing an ROC curve obtained as a result of determining a healthy subject and a pre-sarcopenia subject using the prediction model in the third modification of the present embodiment;

FIG. 18 is a view showing an ROC curve obtained as a result of determining a healthy subject and a sarcopenia subject using a prediction model in a fourth modification of the present embodiment;

FIG. 19 is a view showing an ROC curve obtained as a result of determining a healthy subject and a pre-sarcopenia subject using the prediction model in the fourth modification of the present embodiment;

FIG. 20 is a view showing a vertical displacement of the toe of one foot in one walking cycle in a fifth modification of the present embodiment;

FIG. 21 is a view showing an ROC curve obtained as a result of determining whether or not to be a sarcopenia subject or a pre-sarcopenia subject using a prediction model in the fifth modification of the present embodiment;

FIG. 22 is a view showing an average of mean values of time series data of the vertical displacement of the toe of one foot of sarcopenia subjects or pre-sarcopenia subjects and an average of mean values of time series data of the vertical displacement of the toe of one foot of healthy subjects in the fifth modification of the present embodiment;

FIG. 23 is a view showing a change in the angle of the ankle joint of one foot in one walking cycle in a sixth modification of the present embodiment;

FIG. 24 is a view showing an ROC curve obtained as a result of determining a healthy subject and a sarcopenia subject using a prediction model in the sixth modification of the present embodiment;

FIG. 25 is a view showing an ROC curve obtained as a result of determining a healthy subject and a pre-sarcopenia subject using the prediction model in the sixth modification of the present embodiment;

FIG. 26 is a view showing an ROC curve obtained as a result of determining a healthy subject and a sarcopenia subject using a prediction model in a seventh modification of the present embodiment;

FIG. 27 is a view showing an ROC curve obtained as a result of determining a healthy subject and a pre-sarcopenia subject using the prediction model in the seventh modification of the present embodiment;

FIG. 28 is a view showing an ROC curve obtained as a result of determining a healthy subject and a sarcopenia subject using a prediction model in an eighth modification of the present embodiment;

FIG. 29 is a view showing an ROC curve obtained as a result of determining a healthy subject and a sarcopenia subject using a prediction model in a ninth modification of the present embodiment;

FIG. 30 is a view showing an average of stride distances of one leg of sarcopenia subjects and an average of stride distances of one leg of healthy subjects in the ninth modification of the present embodiment;

FIG. 31 is a view showing an ROC curve obtained as a result of determining a healthy subject and a sarcopenia subject using a prediction model in a tenth modification of the present embodiment;

FIG. 32 is a view showing an ROC curve obtained as a result of determining a healthy subject and a pre-sarcopenia subject using the prediction model in an eleventh modification of the present embodiment;

FIG. 33 is a view showing a change in the angle of the knee joint of one leg in one walking cycle in a twelfth modification of the present embodiment;

FIG. 34 is a view showing an ROC curve obtained as a result of determining whether or not to be a sarcopenia subject or a pre-sarcopenia subject using a prediction model in the twelfth modification of the present embodiment;

FIG. 35 is a view showing an ROC curve obtained as a result of determining a healthy subject and a sarcopenia subject using a prediction model in a thirteenth modification of the present embodiment;

FIG. 36 is a view showing an ROC curve obtained as a result of determining a healthy subject and a pre-sarcopenia subject using the prediction model in a fourteenth modification of the present embodiment;

FIG. 37 is a view showing an ROC curve obtained as a result of determining a healthy subject and a sarcopenia subject using a prediction model in a fifteenth modification of the present embodiment;

FIG. 38 is a view showing an ROC curve obtained as a result of determining a healthy subject and a pre-sarcopenia subject using the prediction model in a sixteenth modification of the present embodiment;

FIG. 39 is a view showing an ROC curve obtained as a result of determining a healthy subject and a sarcopenia subject using a prediction model in a seventeenth modification of the present embodiment;

FIG. 40 is a view showing an ROC curve obtained as a result of determining a healthy subject and a pre-sarcopenia subject using the prediction model in an eighteenth modification of the present embodiment; and

FIG. 41 is a view showing an example of an evaluation result screen displayed in the present embodiment.

### DESCRIPTION OF EMBODIMENTS

(Findings on which the Present Disclosure is Based)

A sheet type pressure sensor or a three-dimensional motion analysis system is used for measurement of a walking parameter in Japanese Patent Application Laid-Open No. 2013-255786. The sheet type pressure sensor measures a pressure distribution at the time of walking, and measures a walking parameter from the pressure distribution. A three-dimensional motion analysis system measures a walking parameter by acquiring, from a plurality of video cameras, image information in which a marker attached on a foot is captured, and analyzing the motion from the image information. It requires a great amount of time and effort to install such a sheet type pressure sensor or a three-dimensional motion analysis system. Therefore, with Japanese Patent Application Laid-Open No. 2013-255786, it is difficult to easily evaluate the senile disorder risk.

Furthermore, walking parameters used in Japanese Patent Application Laid-Open No. 2013-255786 are two or more selected from a cadence, a stride, a walking ratio, a step, a walking interval, a walking angle, a toe angle, a stride right-and-left difference, a walking interval right-and-left difference, a walking angle right-and-left difference, and both legs support period right-and-left difference. The walking angle is an angle formed by a straight line connecting one of the right and left heels with the other heel and the travel direction. The toe angle is an angle formed by a straight line connecting the heel with the toe and the travel direction. Furthermore, in Japanese Patent Application Laid-Open No. 2013-255786, the senile disorder risk of a senile disorder selected from at least knee pain, lower back pain, incontinence of urine, dementia, and sarcopenia is evaluated. However, Japanese Patent Application Laid-Open No. 2013-255786 does not disclose evaluating a senile disorder risk using another walking parameter, and there is a possibility that the use of another walking parameter further improves the evaluation accuracy of the senile disorder risk.

The moving ability evaluation device in Japanese Patent Application Laid-Open No. 2018-114319 evaluates at least one of longitudinal balance, body weight movement, and lateral balance when a subject is moving, from longitudinal acceleration, lateral acceleration, and vertical acceleration when the subject is moving. However, Japanese Patent Application Laid-Open No. 2018-114319 does not disclose evaluating sarcopenia using another parameter, and there is a possibility that the use of another walking parameter further improves the evaluation accuracy of sarcopenia.

In order to solve the above problems, the sarcopenia evaluation method according to an aspect of the present disclosure is a sarcopenia evaluation method in a sarcopenia evaluation device that evaluates sarcopenia based on the walking motion of a subject, the sarcopenia evaluation method including: acquiring walking data related to walking of the subject; detecting, from the walking data, at least one of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of an ankle joint of the one foot in the stance phase, and an angle of the ankle joint of the one foot in the swing phase; and determining whether or not the subject has sarcopenia using at least one of the angle of the knee joint in the stance phase, the angle of the knee joint in the swing phase, the vertical displacement of the toe in the stance phase, the vertical displacement of the toe in the swing phase, the angle of the ankle joint in the stance phase, and the angle of the ankle joint in the swing phase.

According to this configuration, at least one of an angle of the knee joint of one leg in a stance phase of the one leg of a walking subject, an angle of the knee joint of one leg in a swing phase of the one leg, a vertical displacement of the toe of one foot in the stance phase, a vertical displacement of the toe of one foot in the swing phase, an angle of the ankle joint of one foot in the stance phase, and an angle of the ankle joint of one foot in the swing phase is used as a parameter correlated with sarcopenia of the subject. Walking motion of subjects with sarcopenia tends to be different from walking motion of subjects without sarcopenia. In this manner, since it is determined whether or not the subject has sarcopenia using a parameter correlated with sarcopenia of the walking subject, the sarcopenia of the subject can be evaluated with high accuracy.

Furthermore, a large-scale device is unnecessary because at least one of an angle of the knee joint of one leg in a stance phase of the one leg of a walking subject, an angle of the knee joint of the one leg in a swing phase of the one leg, a vertical displacement of the toe of the one foot in the stance phase, a vertical displacement of the toe of the one foot in the swing phase, an angle of the ankle joint of the one foot in the stance phase, and an angle of the ankle joint of the one foot in the swing phase can be easily detected from image data obtained by capturing an image of a walking subject, for example. Therefore, the present configuration can easily evaluate sarcopenia of a subject.

In addition, in the sarcopenia evaluation method described above, in the detection, time series data of an angle of the knee joint in a predetermined period of the swing phase may be detected, and in the determination, whether or not the subject has the sarcopenia may be determined by using a mean value of the time series data of the angle of the knee joint.

There is a significant difference in angle of the knee joint of one leg in a predetermined period of the swing phase of the one leg of a walking subject between subjects with sarcopenia and subjects without sarcopenia. Therefore, according to the present configuration, the sarcopenia of the subject can be reliably evaluated by using a mean value of time series data of the angle of the knee joint of one leg in a predetermined period of the swing phase of the one leg of a walking subject.

Furthermore, in the above-described sarcopenia evaluation method, on the condition that a period from when one foot of the subject touches the ground to when the one foot touches the ground again is expressed as one walking cycle and the one walking cycle is expressed by 1% to 100%, the predetermined period may be a period of 61% to 100% of the one walking cycle.

According to the present configuration, the period from when one foot of the subject touches the ground to when the one foot touches the ground again is expressed as one walking cycle, and one walking cycle is expressed as 1% to 100%. At this time, the sarcopenia of the subject can be reliably evaluated by using a mean value of time series data of the angle of the knee joint of one leg in a period of 61% to 100% of one walking cycle.

In addition, in the sarcopenia evaluation method described above, in the detection, time series data of an angle of the knee joint in a predetermined period of the stance phase may be detected, and in the determination, whether or not the subject has sarcopenia may be determined by using a mean value of the time series data of the angle of the knee joint.

There is a significant difference in angle of the knee joint of one leg in a predetermined period of the stance phase of the one leg of a walking subject between subjects with sarcopenia and subjects without sarcopenia. Therefore, according to the present configuration, the sarcopenia of the subject can be reliably evaluated by using a mean value of time series data of the angle of the knee joint of one leg in a predetermined period of the stance phase of the one leg of a walking subject.

Furthermore, in the above-described sarcopenia evaluation method, on the condition that a period from when one foot of the subject touches the ground to when the one foot touches the ground again is expressed as one walking cycle and the one walking cycle is expressed by 1% to 100%, the predetermined period may be a period of 50% to 60% of the one walking cycle.

According to the present configuration, the period from when one foot of the subject touches the ground to when the one foot touches the ground again is expressed as one walking cycle, and one walking cycle is expressed as 1% to 100%. At this time, the sarcopenia of the subject can be reliably evaluated by using a mean value of time series data of the angle of the knee joint of one leg in a period of 50% to 60% of one walking cycle.

In addition, in the sarcopenia evaluation method described above, in the detection, whether or not the subject has sarcopenia may be determined by detecting time series data of the vertical displacement of the toe in a predetermined period of the stance phase, and in the determination, by using a mean value of the time series data of the vertical displacement of the toe.

There is a significant difference in vertical displacement of the toe of one foot in a predetermined period of the stance phase of the one leg of a walking subject between subjects with sarcopenia and subjects without sarcopenia. Therefore, according to the present configuration, the sarcopenia of the subject can be reliably evaluated by using a mean value of time series data of the vertical displacement of the toe of one foot in a predetermined period of the stance phase of the one leg of a walking subject.

Furthermore, in the above-described sarcopenia evaluation method, on the condition that a period from when one foot of the subject touches the ground to when the one foot touches the ground again is expressed as one walking cycle and the one walking cycle is expressed by 1% to 100%, the predetermined period may be a period of 1% to 60% of the one walking cycle.

According to the present configuration, the period from when one foot of the subject touches the ground to when the one foot touches the ground again is expressed as one walking cycle, and one walking cycle is expressed as 1% to 100%. At this time, the sarcopenia of the subject can be reliably evaluated by using a mean value of time series data of the vertical displacement of the toe of one foot in a period of 1% to 60% of one walking cycle.

In addition, in the sarcopenia evaluation method described above, in the detection, whether or not the subject has sarcopenia may be determined by detecting time series data of the vertical displacement of the toe in a predetermined period of the swing phase, and in the determination, by using a mean value of the time series data of the vertical displacement of the toe.

There is a significant difference in vertical displacement of the toe of one foot in a predetermined period of the swing phase of the one leg of a walking subject between subjects with sarcopenia and subjects without sarcopenia. Therefore, according to the present configuration, the sarcopenia of the subject can be reliably evaluated by using a mean value of time series data of the vertical displacement of the toe of one foot in a predetermined period of the swing phase of the one leg of a walking subject.

Furthermore, in the above-described sarcopenia evaluation method, on the condition that a period from when one foot of the subject touches the ground to when the one foot touches the ground again is expressed as one walking cycle and the one walking cycle is expressed by 1% to 100%, the predetermined period may be a period of 65% to 70% of the one walking cycle.

According to the present configuration, the period from when one foot of the subject touches the ground to when the one foot touches the ground again is expressed as one walking cycle, and one walking cycle is expressed as 1% to 100%. At this time, the sarcopenia of the subject can be reliably evaluated by using a mean value of time series data of the vertical displacement of the toe of one foot in a period of 65% to 70% of one walking cycle.

In addition, in the sarcopenia evaluation method described above, in the detection, time series data of a first angle of the ankle joint in a first period of the stance phase and time series data of a second angle of the ankle joint in a second period of the swing phase may be detected, and in the determination, whether or not the subject has sarcopenia may be determined by using a mean value of the time series data of the first angle of the ankle joint and a mean value of the time series data of the second angle of the ankle joint.

According to the present configuration, a mean value of time series data of a first angle of the ankle joint of one foot in a first period of the stance phase of the one leg and a mean value of time series data of a second angle of the ankle joint of one foot in a second period of the swing phase of the one leg are used in combination, whereby sarcopenia can be evaluated more accurately than by using each of them in isolation.

In addition, in the sarcopenia evaluation method described above, in the detection, time series data of the vertical displacement of the toe in a first period of the stance phase, time series data of the angle of the knee joint in a second period of the stance phase, time series data of the angle of the knee joint in a third period of the swing phase, and time series data of the angle of the knee joint in a fourth period of the swing phase may be detected, and in the determination, whether or not the subject has sarcopenia may be determined by using a mean value of the time series data of the vertical displacement of the toe in the first period and each of mean values of the time series data of the angles of the knee joint in each of the second period, the third period, and the fourth period.

According to the present configuration, a mean value of time series data of a vertical displacement of the toe of one foot in a first period of the stance phase of the one leg, a mean value of time series data of an angle of the knee joint of one leg in a second period of the stance phase of the one leg, a mean value of time series data of an angle of the knee joint of one leg in a third period of the swing phase of the one leg, and a mean value of time series data of an angle of the knee joint of one leg in a fourth period of the swing phase of the one leg are used in combination, whereby sarcopenia can be evaluated more accurately than by using each of them in isolation.

In addition, in the sarcopenia evaluation method described above, in the detection, time series data of the vertical displacement of the toe in a first period of the stance phase, time series data of the angle of the ankle joint in a second period of the stance phase, time series data of the angle of the ankle joint in a third period of the stance phase, time series data of the angle of the ankle joint in a fourth period of the swing phase, and time series data of the angle of the ankle joint in a fifth period of the swing phase may be detected, and in the determination, whether or not the subject has sarcopenia may be determined by using a mean value of the time series data of the vertical displacement of the toe in the first period and each of mean values of the time series data of the angles of the ankle joint in each of the second period, the third period, the fourth period, and the fifth period.

According to the present configuration, a mean value of time series data of a vertical displacement of the toe of one foot in a first period of the stance phase of the one leg, a mean value of time series data of an angle of the ankle joint of one foot in a second period of the stance phase of the one leg, a mean value of time series data of an angle of the ankle joint of one foot in a third period of the stance phase of the one leg, a mean value of time series data of an angle of the ankle joint of one foot in a fourth period of the swing phase of the one leg, and a mean value of time series data of an angle of the ankle joint of one foot in a fifth period of the swing phase of the one leg are used in combination, whereby sarcopenia can be evaluated more accurately than by using each of them in isolation.

In addition, in the sarcopenia evaluation method described above, in the detection, time series data of the angle of the knee joint in a first period of the stance phase, time series data of the angle of the knee joint in a second period of the swing phase, time series data of the angle of the knee joint in a third period of the swing phase, time series data of the angle of the ankle joint in a fourth period of the stance phase, time series data of the angle of the ankle joint in a fifth period of the swing phase, and time series data of the angle of the ankle joint in a sixth period of the swing phase may be detected, and in the determination, whether or not the subject has sarcopenia may be determined by using each of mean values of the time series data of the angles of the knee joint in each of the first period, the second period, and the third period, and each of mean values of the time series data of the angles of the ankle joint in each of the fourth period, the fifth period, and the sixth period.

According to the present configuration, a mean value of time series data of an angle of the knee joint of one leg in a first period of the stance phase of the one leg, a mean value of time series data of an angle of the knee joint of one leg in a second period of the swing phase of the one leg, a mean value of time series data of an angle of the knee joint of one leg in a third period of the swing phase of the one leg, a mean value of time series data of an angle of the ankle joint of one foot in a fourth period of the stance phase of the one leg, a mean value of time series data of an angle of the ankle joint of one foot in a fifth period of the swing phase of the one leg, and a mean value of time series data of an angle of the ankle joint of one foot in a sixth period of the swing phase of the one leg are used in combination, whereby sarcopenia can be evaluated more accurately than by using each of them in isolation.

In addition, in the sarcopenia evaluation method described above, in the detection, time series data of the vertical displacement of the toe in a first period of the stance phase, time series data of the vertical displacement of the toe in a second period of the stance phase, time series data of the vertical displacement of the toe in a third period of the swing phase, time series data of the angle of the knee joint in a fourth period of the stance phase, time series data of the angle of the knee joint in a fifth period of the stance phase and the swing phase, time series data of the angle of the ankle joint in a sixth period of the stance phase, and time series data of the angle of the ankle joint in a seventh period of the stance phase and the swing phase may be detected, and in the determination, whether or not the subject has sarcopenia may be determined by using each of mean values of the time series data of the vertical displacements of the toe in each of the first period, the second period, and the third period, each of mean values of the time series data of the angles of the knee joint in each of the fourth period and the fifth period, and each of mean values of the time series data of the angles of the ankle joint in each of the sixth period and the seventh period.

According to the present configuration, a mean value of time series data of a vertical displacement of the toe of one foot in a first period of the stance phase of the one leg, a mean value of time series data of a vertical displacement of the toe of one foot in a second period of the stance phase of the one leg, a mean value of time series data of a vertical displacement of the toe of one foot in a third period of the swing phase of the one leg, a mean value of time series data of an angle of the knee joint of one leg in a fourth period of the stance phase of the one leg, a mean value of time series data of an angle of the knee joint of one leg in a fifth period of the stance phase and the swing phase of the one leg, a mean value of time series data of an angle of the ankle joint of one foot in a sixth period of the stance phase of the one leg, and a mean value of time series data of an angle of the ankle joint of one foot in a seventh period of the stance phase and the swing phase of the one leg are used in combination, whereby sarcopenia can be evaluated more accurately than by using each of them in isolation.

Furthermore, in the sarcopenia evaluation method described above, it may be further determined whether or not the subject is a pre-sarcopenia subject, who will potentially have sarcopenia in the future, using at least one of an angle of the knee joint in the stance phase, an angle of the knee joint in the swing phase, the vertical displacement of the toe in the stance phase, the vertical displacement of the toe in the swing phase, an angle of the ankle joint in the stance phase, and an angle of the ankle joint in the swing phase.

According to this configuration, at least one of an angle of the knee joint of one leg in a stance phase of the one leg of a walking subject, an angle of the knee joint of one leg in a swing phase of the one leg, a vertical displacement of the toe of one foot in the stance phase, a vertical displacement of the toe of one foot in the swing phase, an angle of the ankle joint of one foot in the stance phase, and an angle of the ankle joint of one foot in the swing phase is used as a parameter correlated with sarcopenia of the subject. Walking motion of a subject who is a pre-sarcopenia subject, who will potentially have sarcopenia in the future, tends to be different from walking motion of a subject who is not a pre-sarcopenia subject. In this manner, since it is determined whether or not the subject is a pre-sarcopenia subject by using a parameter correlated with sarcopenia of the walking subject, the sarcopenia of the subject can be evaluated with high accuracy.

Furthermore, in the above-described sarcopenia evaluation method, in the determination, when an angle of the knee joint in the stance phase is larger than a threshold value, when an angle of the knee joint in the swing phase is larger than a threshold value, when the vertical displacement of the toe in the stance phase is larger than a threshold value, when the vertical displacement of the toe in the swing phase is larger than a threshold value, when an angle of the ankle joint in the stance phase is larger than a threshold value, or when an angle of the ankle joint in the swing phase is larger than a threshold value, it may be determined that the subject has the sarcopenia.

According to this configuration, when an angle of the knee joint of one leg in the stance phase of the one leg is larger than a threshold value, when an angle of the knee joint of one leg in the swing phase of the one leg is larger than the threshold value, when a vertical displacement of the toe of one foot in the stance phase of the one leg is larger than a threshold value, when a vertical displacement of the toe of one foot in the swing phase of the one leg is larger than the threshold value, when an angle of the ankle joint of one foot in the stance phase of the one leg is larger than a threshold value, or when an angle of the ankle joint of one foot in the swing phase of the one leg is larger than the threshold value, it is determined that the subject has sarcopenia.

Accordingly, it is possible to easily determine whether or not the subject has sarcopenia by comparing, with a threshold value, an angle of the knee joint of one leg in the stance phase of the one leg, an angle of the knee joint of one leg in the swing phase of the one leg, a vertical displacement of the toe of one foot in the stance phase of the one leg, a vertical displacement of the toe of one foot in the swing phase of the one leg, an angle of the ankle joint of one foot in the stance phase of the one leg, or an angle of the ankle joint of one foot in the swing phase of the one leg.

Furthermore, in the above-described sarcopenia evaluation method, in the determination, whether or not the subject has the sarcopenia may be determined by inputting at least one of an angle of the knee joint in the stance phase, an angle of the knee joint in the swing phase, the vertical displacement of the toe in the stance phase, the vertical displacement of the toe in the swing phase, an angle of the ankle joint in the stance phase, and an angle of the ankle joint in the swing phase that has been detected into a prediction model generated with at least one of an angle of the knee joint in the stance phase, an angle of the knee joint in the swing phase, the vertical displacement of the toe in the stance phase, the vertical displacement of the toe in the swing phase, an angle of the ankle joint in the stance phase, and an angle of the ankle joint in the swing phase as an input value, and with whether or not the subject has the sarcopenia as an output value.

According to the present configuration, the prediction model is generated with at least one of an angle of the knee joint of one leg in the stance phase of the one leg, an angle of the knee joint of one leg in the swing phase of the one leg, a vertical displacement of the toe of one foot in the stance phase of the one leg, a vertical displacement of the toe of one foot in the swing phase of the one leg, an angle of the ankle joint of one foot in the stance phase of the one leg, and an angle of the ankle joint of one foot in the swing phase of the one leg as an input value, and whether or not the subject has sarcopenia as an output value. Then, whether or not the subject has sarcopenia is determined by inputting, into the prediction model, at least one of an angle of the knee joint of one leg in the stance phase of the one leg, an angle of the knee joint of one leg in the swing phase of the one leg, a vertical displacement of the toe of one foot in the stance phase of the one leg, a vertical displacement of the toe of one foot in the swing phase of the one leg, an angle of the ankle joint of one foot in the stance phase of the one leg, and an angle of the ankle joint of one foot in the swing phase of the one leg that has been detected. Accordingly, it is possible to easily determine whether or not the subject has sarcopenia by storing the prediction model in advance.

A sarcopenia evaluation device according to another aspect of the present disclosure is a sarcopenia evaluation device that evaluates sarcopenia based on the walking motion of a subject, the sarcopenia evaluation device including: an acquisition unit that acquires walking data related to walking of the subject; a detection unit that detects, from the walking data, at least one of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of the ankle joint of the one foot in the stance phase, and an angle of the ankle joint of the one foot in the swing phase; and a determination unit that determines whether or not the subject has sarcopenia using at least one of the angle of the knee joint in the stance phase, the angle of the knee joint in the swing phase, the vertical displacement of the toe in the stance phase, the vertical displacement of the toe in the swing phase, the angle of the ankle joint in the stance phase, and the angle of the ankle joint in the swing phase.

According to this configuration, at least one of an angle of the knee joint of one leg in a stance phase of the one leg of a walking subject, an angle of the knee joint of one leg in a swing phase of the one leg, a vertical displacement of the toe of one foot in the stance phase, a vertical displacement of the toe of one foot in the swing phase, an angle of the ankle joint of one foot in the stance phase, and an angle of the ankle joint of one foot in the swing phase is used as a parameter correlated with sarcopenia of the subject. Walking motion of subjects with sarcopenia tends to be different from walking motion of subjects without sarcopenia. In this manner, since it is determined whether or not the subject has sarcopenia using a parameter correlated with sarcopenia of the walking subject, the sarcopenia of the subject can be evaluated with high accuracy.

Furthermore, a large-scale device is unnecessary because at least one of an angle of the knee joint of one leg in a stance phase of the one leg of a walking subject, an angle of the knee joint of the one leg in a swing phase of the one leg, a vertical displacement of the toe of the one foot in the stance phase, a vertical displacement of the toe of the one foot in the swing phase, an angle of the ankle joint of the one foot in the stance phase, and an angle of the ankle joint of the one foot in the swing phase can be easily detected from image data obtained by capturing an image of a walking subject, for example. Therefore, the present configuration can easily evaluate sarcopenia of a subject.

A non-transitory computer-readable recording medium in which a sarcopenia evaluation program is recorded according to another aspect of the present disclosure is a non-transitory computer-readable recording medium in which a sarcopenia evaluation program that evaluates sarcopenia based on walking motion of a subject is recorded, in which the non-transitory computer-readable recording medium causes a computer to function so as to acquire walking data related to walking of the subject, so as to detect, from the walking data, at least one of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of the ankle joint of the one foot in the stance phase, and an angle of the ankle joint of the one foot in the swing phase, and so as to determine whether or not the subject has sarcopenia using at least one of the angle of the knee joint in the stance phase, the angle of the knee joint in the swing phase, a vertical displacement of the toe in the stance phase, a vertical displacement of the toe in the swing phase, the angle of the ankle joint in the stance phase, and the angle of the ankle joint in the swing phase.

According to this configuration, at least one of an angle of the knee joint of one leg in a stance phase of the one leg of a walking subject, an angle of the knee joint of one leg in a swing phase of the one leg, a vertical displacement of the toe of one foot in the stance phase, a vertical displacement of the toe of one foot in the swing phase, an angle of the ankle joint of one foot in the stance phase, and an angle of the ankle joint of one foot in the swing phase is used as a parameter correlated with sarcopenia of the subject. Walking motion of subjects with sarcopenia tends to be different from walking motion of subjects without sarcopenia. In this manner, since it is determined whether or not the subject has sarcopenia using a parameter correlated with sarcopenia of the walking subject, the sarcopenia of the subject can be evaluated with high accuracy.

Furthermore, a large-scale device is unnecessary because at least one of an angle of the knee joint of one leg in a stance phase of the one leg of a walking subject, an angle of the knee joint of the one leg in a swing phase of the one leg, a vertical displacement of the toe of the one foot in the stance phase, a vertical displacement of the toe of the one foot in the swing phase, an angle of the ankle joint of the one foot in the stance phase, and an angle of the ankle joint of the one foot in the swing phase can be easily detected from image data obtained by capturing an image of a walking subject, for example. Therefore, the present configuration can easily evaluate sarcopenia of a subject.

An embodiment of the present disclosure will now be described with reference to the accompanying drawings. It is to be noted that the following embodiment is an example embodying the present disclosure, and does not limit the technical scope of the present disclosure.

### Embodiment

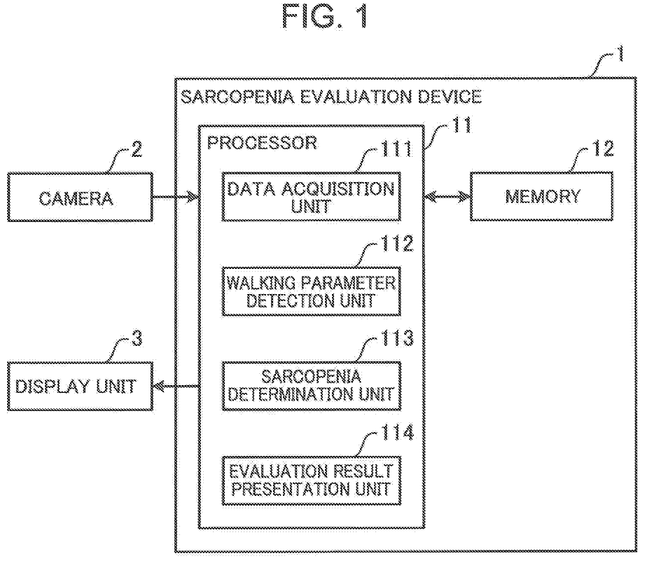

A sarcopenia evaluation system according to the present embodiment will be described below with reference to FIG. 1.

FIG. 1 is a block diagram showing a configuration of a sarcopenia evaluation system in an embodiment of the present disclosure.

The sarcopenia evaluation system shown in FIG. 1 includes a sarcopenia evaluation device 1, a camera 2, and a display unit 3.

The camera 2 captures an image of a walking subject. The camera 2 outputs moving image data showing a walking subject to the sarcopenia evaluation device 1. The camera 2 is connected with the sarcopenia evaluation device 1 by wire or wirelessly.

The sarcopenia evaluation device 1 includes a processor 11 and a memory 12.

The processor 11 is, for example, a central processing unit (CPU), and includes a data acquisition unit 111, a walking parameter detection unit 112, a sarcopenia determination unit 113, and an evaluation result presentation unit 114.

The memory 12 is a storage device capable of storing various kinds of information, such as a random access memory (RAM), a hard disk drive (HDD), a solid state drive (SSD), or a flash memory.

The data acquisition unit 111 acquires walking data related to walking of the subject. The walking data is moving image data obtained by capturing an image of a walking subject, for example. The data acquisition unit 111 acquires moving image data having been output by the camera 2.

The walking parameter detection unit 112 extracts skeleton data showing the skeleton of the subject from moving image data acquired by the data acquisition unit 111. The skeleton data is represented by coordinates of a plurality of feature points indicating the joints and the like of the subject and straight lines connecting the feature points. The walking parameter detection unit 112 may use software (e.g., OpenPose or 3D-pose-baseline) that detects the coordinates of feature points of a person from two-dimensional image data.

The processing of extracting skeleton data from two-dimensional image data will now be described.

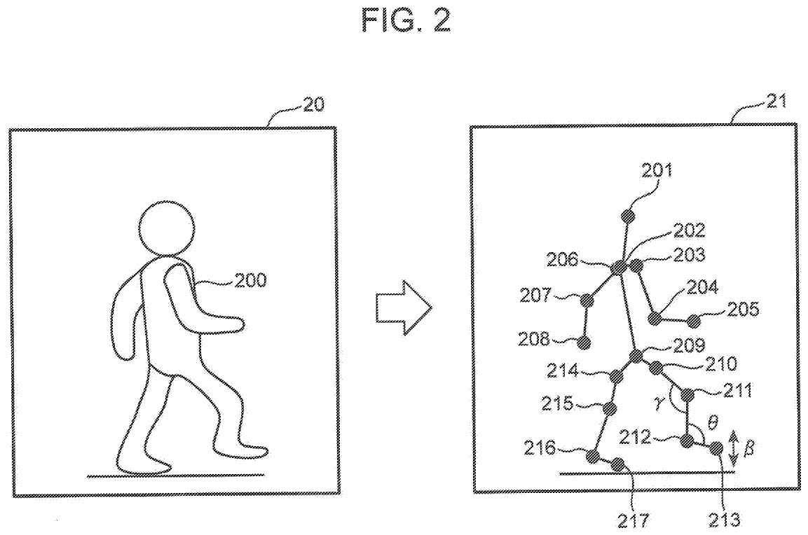

FIG. 2 is a view for explaining processing of extracting skeleton data from two-dimensional image data in the present embodiment.

The walking parameter detection unit 112 extracts skeleton data 21 from two-dimensional image data 20 including an image of a walking subject 200. The skeleton data 21 includes a feature point 201 indicating the head, a feature point 202 indicating the center of both shoulders, a feature point 203 indicating the right shoulder, a feature point 204 indicating the right elbow, a feature point 205 indicating the right hand, a feature point 206 indicating the left shoulder, a feature point 207 indicating the left elbow, a feature point 208 indicating the left hand, a feature point 209 indicating the waist, a feature point 210 indicating the right hip joint, a feature point 211 indicating the right knee joint, a feature point 212 indicating the right ankle joint, a feature point 213 indicating the right toe, a feature point 214 indicating the left hip joint, a feature point 215 indicating the left knee joint, a feature point 216 indicating the left ankle joint, and a feature point 217 indicating the left toe.

The moving image data is composed of a plurality of two-dimensional image data. The walking parameter detection unit 112 extracts time series skeleton data from each of a plurality of two-dimensional image data constituting moving image data. It is to be noted that the walking parameter detection unit 112 may extract skeleton data from two-dimensional image data of all frames or may extract skeleton data from two-dimensional image data of each predetermined frame. In addition, in the present embodiment, sarcopenia is evaluated based on the movement of mainly the lower limbs of the walking subject. Therefore, the walking parameter detection unit 112 may extract only the skeleton data of the lower limbs of the subject.

In addition, the walking parameter detection unit 112 clips skeleton data corresponding to one walking cycle of the subject from time series skeleton data extracted from moving image data. The human walking motion is a cyclic motion.

The walking cycle of the subject will now be described.

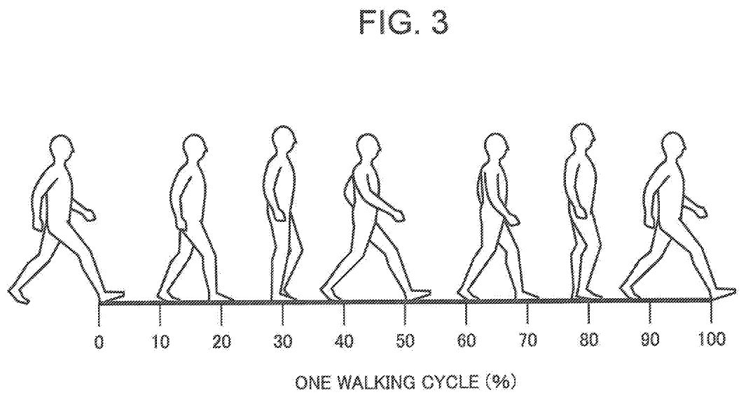

FIG. 3 is a view for explaining a walking cycle in the present embodiment.

As shown in FIG. 3, the period from when one foot of the subject touches the ground to when the one foot touches the ground again is expressed as one walking cycle. The one walking cycle shown in FIG. 3 is a period from when the right foot of the subject touches the ground to when the right foot touches the ground again. In addition, one walking cycle is normalized to 1% to 100%. The period of 1% to 60% of one walking cycle is called a stance phase in which one foot (e.g., right foot) is on the ground, and the period of 61% to 100% of one walking cycle is called a swing phase in which one foot (e.g., right foot) is off the ground. One walking cycle includes the stance phase and the swing phase. It is to be noted that one walking cycle may be a period from when the left foot of the subject touches the ground to when the left foot touches the ground again.

The walking parameter detection unit 112 detects, from walking data, at least one of an angle of the knee joint of one leg in a stance phase of the one leg of a subject, an angle of the knee joint of one leg in a swing phase of the one leg, a vertical displacement of the toe of one foot in the stance phase, a vertical displacement of the toe of one foot in the swing phase, an angle of the ankle joint of one foot in the stance phase, and an angle of the ankle joint of one foot in the swing phase.

In the present embodiment, the walking parameter detection unit 112 detects, from walking data, the angle of the knee joint in the swing phase of one leg of the subject. The walking parameter detection unit 112 detects the angle of the knee joint in the swing phase of one leg of the subject from the time series skeleton data corresponding to the one walking cycle having been clipped. As shown in FIG. 2, an angle γ of the knee joint is an angle formed in the sagittal plane by a straight line connecting the feature point 211 indicating the right knee joint and the feature point 210 indicating the right hip joint and a straight line connecting the feature point 211 indicating the right knee joint and the feature point 212 indicating the right ankle joint.

In particular, the walking parameter detection unit 112 detects time series data of the angle of the knee joint of one leg in a predetermined period of the swing phase of one leg. More specifically, the predetermined period is a period of 61% to 100% of one walking cycle. The walking parameter detection unit 112 calculates, as a walking parameter, a mean value of time series data of the angle of the knee joint of one leg in a predetermined period of the swing phase of the one leg.

It is to be noted that detection of an angle of the knee joint of one leg in the stance phase of the one leg of a subject, a vertical displacement of the toe of one foot in the stance phase, a vertical displacement of the toe of one foot in the swing phase, an angle of the ankle joint of one foot in the stance phase, and an angle of the ankle joint of one foot in the swing phase will be described in modifications of the present embodiment.

The sarcopenia determination unit 113 determines whether or not the subject has sarcopenia using at least one of an angle of the knee joint of one leg in the stance phase, an angle of the knee joint of one leg in the swing phase, a vertical displacement of the toe of one foot in the stance phase, a vertical displacement of the toe of one foot in the swing phase, an angle of the ankle joint of one foot in the stance phase, and an angle of the ankle joint of one foot in the swing phase.

In the present embodiment, the sarcopenia determination unit 113 determines whether or not the subject has sarcopenia using the mean value of time series data of the angle of the knee joint of one leg in the swing phase.

In addition, the sarcopenia determination unit 113 determines whether or not the subject has sarcopenia by inputting at least one of an angle of the knee joint of one leg in the stance phase, an angle of the knee joint of one leg in the swing phase, the vertical displacement of the toe of one foot in the stance phase, the vertical displacement of the toe of one foot in the swing phase, an angle of the ankle joint of one foot in the stance phase, and an angle of the ankle joint of one foot in the swing phase that has been detected into a prediction model generated with at least one of an angle of the knee joint of one leg in the stance phase, an angle of the knee joint of one leg in the swing phase, the vertical displacement of the toe of one foot in the stance phase, the vertical displacement of the toe of one foot in the swing phase, an angle of the ankle joint of one foot in the stance phase, and an angle of the ankle joint of one foot in the swing phase as an input value, and with whether or not the subject has sarcopenia as an output value.

In the present embodiment, the sarcopenia determination unit 113 determines whether or not the subject has sarcopenia by inputting an angle of the knee joint of one leg in the swing phase that has been detected by the walking parameter detection unit 112 into a prediction model generated with an angle of the knee joint of one leg in the swing phase as an input value, and with whether or not the subject has sarcopenia as an output value.

It is to be noted that determination of sarcopenia of the subject using an angle of the knee joint of one leg in the stance phase, a vertical displacement of the toe of one foot in the stance phase, a vertical displacement of the toe of one foot in the swing phase, an angle of the ankle joint of one foot in the stance phase, and an angle of the ankle joint of one foot in the swing phase will be described in modifications of the present embodiment.

In addition, in the present embodiment, the sarcopenia determination unit 113 does not only determine whether or not the subject has sarcopenia. The sarcopenia determination unit 113 determines whether or not the subject is a pre-sarcopenia subject, who will potentially have sarcopenia in the future, using at least one of an angle of the knee joint of one leg in the stance phase, an angle of the knee joint of one leg in the swing phase, a vertical displacement of the toe of one foot in the stance phase, a vertical displacement of the toe of one foot in the swing phase, an angle of the ankle joint of one foot in the stance phase, and an angle of the ankle joint of one foot in the swing phase. That is, the sarcopenia determination unit 113 determines as to which of a sarcopenia subject, a pre-sarcopenia subject, and a healthy subject the subject is.

Sarcopenia refers to a condition in which the muscle mass of the subject has decreased and the muscle strength or physical ability has decreased. Pre-sarcopenia refers to a condition in which the muscle strength and physical ability have not decreased but only the muscle mass of the subject has decreased. The muscle mass is obtained by measuring limb skeletal muscle mass of the subject, for example. When the limb skeletal muscle mass is lower than a threshold value, it can be judged that the muscle mass has decreased. In addition, the muscle strength is obtained by measuring the grip strength of the subject, for example. When the grip strength is lower than a threshold value, it can be judged that the muscle strength has decreased. The physical ability is obtained by measuring the walking speed of the subject, for example. When the walking speed is lower than a threshold value, it can be judged that the physical ability has decreased.

The memory 12 stores in advance a prediction model generated with the angle of the knee joint of one leg in the swing phase as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the time series data of the angle of the knee joint of one leg in a predetermined period of the swing phase of one walking cycle as an explanatory variable. The prediction model outputs either a value (e.g., 2) indicating that the subject has sarcopenia, a value (e.g., 1) indicating that the subject is a pre-sarcopenia subject, or a value (e.g., 0) indicating that the subject is neither a sarcopenia subject nor a pre-sarcopenia subject, i.e., the subject is a healthy subject.

In particular, the sarcopenia determination unit 113 determines which of a sarcopenia subject, a pre-sarcopenia subject, and a healthy subject the subject is by using the mean value of time series data of the angle of the knee joint in the swing phase of one leg. More specifically, the sarcopenia determination unit 113 determines which of a sarcopenia subject, a pre-sarcopenia subject, and a healthy subject the subject is by using the mean value of time series data of the angle of the knee joint of one leg in a period of 61% to 100% of one walking cycle.

It is to be noted that the prediction model may be generated by machine learning. The machine learning includes, for example, supervised learning for learning the relationship between input and output by using training data in which a label (output information) is given to input information, unsupervised learning for constructing a structure of data only from an unlabeled input, semi-supervised learning for handling both the labeled and the unlabeled, and reinforcement learning for learning, on a trial-and-error basis, a behavior that maximizes reward. Specific methods of machine learning include a neural network (including deep learning using a multilayer neural network), genetic programming, a decision tree, a Bayesian network, and support vector machine (SVM). In the machine learning of the present disclosure, any of the above specific examples may be used.

In addition, the prediction model may output a value indicating the possibility that the subject has sarcopenia. The value indicating the possibility that the subject has sarcopenia is represented by 0.0 to 2.0, for example. In that case, for example, the sarcopenia determination unit 113 may determine that the subject is a healthy subject when the value indicating the possibility that the subject has sarcopenia is equal to or less than 0.5, determine that the subject is a pre-sarcopenia subject when the value indicating the possibility that the subject has sarcopenia is larger than 0.5 and equal to or less than 1.5, and determine that the subject has sarcopenia when the value indicating the possibility that the subject has sarcopenia is larger than 1.5.

The memory 12 may store a first prediction model for determining whether or not the subject has sarcopenia and a second prediction model for determining whether or not the subject is a pre-sarcopenia subject. In this case, the sarcopenia determination unit 113 determines whether or not the subject has sarcopenia by inputting, to the first prediction model, at least one of an angle of the knee joint of one leg in the stance phase, an angle of the knee joint of one leg in the swing phase, a vertical displacement of the toe of one foot in the stance phase, a vertical displacement of the toe of one foot in the swing phase, an angle of the ankle joint of one foot in the stance phase, and an angle of the ankle joint of one foot in the swing phase. When determining that the subject has no sarcopenia, the sarcopenia determination unit 113 determines whether or not the subject is a pre-sarcopenia subject by inputting, to the second prediction model, at least one of an angle of the knee joint of one leg in the stance phase, an angle of the knee joint of one leg in the swing phase, a vertical displacement of the toe of one foot in the stance phase, a vertical displacement of the toe of one foot in the swing phase, an angle of the ankle joint of one foot in the stance phase, and an angle of the ankle joint of one foot in the swing phase. When determining that the subject is not a pre-sarcopenia subject, the sarcopenia determination unit 113 determines that the subject is a healthy subject.

The evaluation result presentation unit 114 presents the evaluation result of the sarcopenia determined by the sarcopenia determination unit 113. The evaluation result presentation unit 114 outputs to the display unit 3 the evaluation result determined by the sarcopenia determination unit 113. The evaluation result is at least one of information indicating whether or not the subject determined by the sarcopenia determination unit 113 has sarcopenia and an evaluation message. It is to be noted that the evaluation result presentation unit 114 may present an evaluation result indicating which of a sarcopenia subject, a pre-sarcopenia subject, and a healthy subject the subject determined by the sarcopenia determination unit 113 is.

The display unit 3 displays the evaluation result having been output from the evaluation result presentation unit 114. The display unit 3 is, for example, a liquid crystal display panel or a light emitting element.

It is to be noted that in order to compare the value indicating the possibility that the currently determined subject has sarcopenia with the value indicating the possibility that a past subject has sarcopenia, the display unit 3 may display a graph of transition of the value indicating the possibility that the subject has sarcopenia. It is to be noted that the value indicating the possibility that the past subject has sarcopenia is stored in the memory 12 and is read from the memory 12.

It is to be noted that the sarcopenia evaluation device 1 may include the camera 2 and the display unit 3. The sarcopenia evaluation device 1 may include the display unit 3. The sarcopenia evaluation device 1 may be a personal computer or a server.

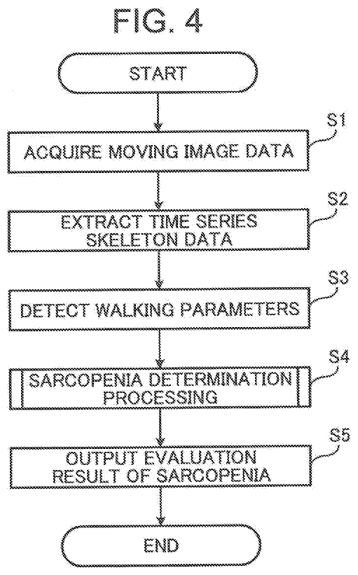

Next, the sarcopenia evaluation processing in the present embodiment will be described with reference to FIG. 4.

FIG. 4 is a flowchart for explaining the sarcopenia evaluation processing using the walking motion of a subject in the present embodiment. The flowchart shown in FIG. 4 shows a procedure of evaluation of sarcopenia using the sarcopenia evaluation device 1.

The subject walks in front of the camera 2. The camera 2 captures an image of the walking subject. The camera 2 transmits moving image data of the walking subject to the sarcopenia evaluation device 1.

First, in step S1, the data acquisition unit 111 acquires the moving image data transmitted by the camera 2.

Next, in step S2, the walking parameter detection unit 112 extracts time series skeleton data from the moving image data.

Next, in step S3, the walking parameter detection unit 112 detects a walking parameter for determining sarcopenia from the time series skeleton data. Here, the walking parameter in the present embodiment is a mean value of the time series data of the angle of the knee joint of one leg in a predetermined period of the swing phase of the one leg in one walking cycle. The predetermined period is a period of 61% to 100% of one walking cycle, for example. A decision method of the walking parameter will be described later.

Next, in step S4, the sarcopenia determination unit 113 executes the sarcopenia determination processing for determining which of a sarcopenia subject, a pre-sarcopenia subject, and a healthy subject the subject is by using the walking parameter. It is to be noted that the sarcopenia determination processing will be described later.

Next, in step S5, the evaluation result presentation unit 114 outputs to the display unit 3 the evaluation result of the sarcopenia determined by the sarcopenia determination unit 113. The sarcopenia evaluation result indicates which of a sarcopenia subject, a pre-sarcopenia subject, or a healthy subject the subject is. It is to be noted that the evaluation result presentation unit 114 may output to the display unit 3 not only which of a sarcopenia subject, a pre-sarcopenia subject, and a healthy subject but also an evaluation message associated with a sarcopenia subject, a pre-sarcopenia subject, or a healthy subject. The display unit 3 displays the sarcopenia evaluation result having been output from the evaluation result presentation unit 114.

The sarcopenia determination processing in step S4 of FIG. 4 will now be described.

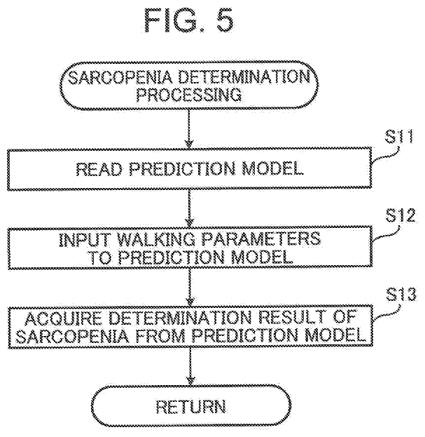

FIG. 5 is a flowchart for explaining the sarcopenia determination processing in step S4 of FIG. 4.

First, in step S11, the sarcopenia determination unit 113 reads the prediction model from the memory 12.

Next, in step S12, the sarcopenia determination unit 113 inputs to the prediction model the walking parameter detected by the walking parameter detection unit 112. The walking parameter in the present embodiment is a mean value of the time series data of the angle of the knee joint of one leg of the subject in a period of 61% to 100% of one walking cycle. The sarcopenia determination unit 113 inputs to the prediction model a mean value of the time series data of the angle of the knee joint of one leg of the subject in the period of 61% to 100% of one walking cycle.

Next, in step S13, the sarcopenia determination unit 113 acquires a sarcopenia determination result from the prediction model. The sarcopenia determination unit 113 obtains, from a prediction model, as a determination result, which of a sarcopenia subject, a pre-sarcopenia subject, and a healthy subject the subject is.

It is to be noted that in the sarcopenia determination processing of the present embodiment, by inputting a walking parameter to a prediction model generated in advance, it is determined which of a sarcopenia subject, a pre-sarcopenia subject, and a healthy subject the subject is. However, the present disclosure is not particularly limited thereto. In another example of the sarcopenia determination processing of the present embodiment, by comparing a threshold value stored in advance with a walking parameter, it may be determined which of a sarcopenia subject, a pre-sarcopenia subject, and a healthy subject the subject is.

In this case, the memory 12 stores in advance a first threshold value for determining whether or not the subject has sarcopenia and a second threshold value for determining whether or not the subject is a pre-sarcopenia subject. The second threshold value is smaller than the first threshold value.

In addition, the sarcopenia determination unit 113 may determine that the subject has sarcopenia when the angle of the knee joint of one leg in the stance phase is larger than the first threshold value, when the angle of the knee joint of one leg in the swing phase is larger than the first threshold value, when the vertical displacement of the toe of one foot in the stance phase is larger than the first threshold value, when the vertical displacement of the toe of one foot in the swing phase is larger than the first threshold value, when the angle of the ankle joint of one foot in the stance phase is larger than the first threshold value, or when the angle of the ankle joint of one foot in the swing phase is larger than the first threshold value.

In the present embodiment, the sarcopenia determination unit 113 may determine that the subject has sarcopenia when the angle of the knee joint of one leg in the swing phase is larger than the first threshold value. The sarcopenia determination unit 113 determines whether or not the mean value of time series data of the angle of the knee joint of one leg of the subject in a period of 61% to 100% of one walking cycle is larger than the first threshold value. The sarcopenia determination unit 113 determines that the subject has sarcopenia when the mean value of the time series data of the angle of the knee joint of the one leg of the subject in the period of 61% to 100% of one walking cycle is larger than the first threshold value.

On the other hand, when the angle of the knee joint of one leg in the stance phase is equal to or less than the first threshold value, the sarcopenia determination unit 113 may determine whether or not the angle of the knee joint of the one leg in the stance phase is larger than the second threshold value. In addition, when the angle of the knee joint of one leg in the swing phase is equal to or less than the first threshold value, the sarcopenia determination unit 113 may determine whether or not the angle of the knee joint of the one leg in the swing phase is larger than the second threshold value. In addition, when the vertical displacement of the toe of one foot in the stance phase is equal to or less than the first threshold value, the sarcopenia determination unit 113 may determine whether or not the vertical displacement of the toe of the one foot in the stance phase is larger than the second threshold value. In addition, when the vertical displacement of the toe of one foot in the swing phase is equal to or less than the first threshold value, the sarcopenia determination unit 113 may determine whether or not the vertical displacement of the toe of the one foot in the swing phase is larger than the second threshold value. In addition, when the angle of the ankle joint of one foot in the stance phase is equal to or less than the first threshold value, the sarcopenia determination unit 113 may determine whether or not the angle of the ankle joint of the one foot in the stance phase is larger than the second threshold value. In addition, when the angle of the ankle joint of one foot in the swing phase is equal to or less than the first threshold value, the sarcopenia determination unit 113 may determine whether or not the angle of the ankle joint of the one foot in the swing phase is larger than the second threshold value.

In addition, the sarcopenia determination unit 113 may determine that the subject is a pre-sarcopenia subject when the angle of the knee joint of one leg in the stance phase is larger than the second threshold value, when the angle of the knee joint of one leg in the swing phase is larger than the second threshold value, when the vertical displacement of the toe of one foot in the stance phase is larger than the second threshold value, when the vertical displacement of the toe of one foot in the swing phase is larger than the second threshold value, when the angle of the ankle joint of one foot in the stance phase is larger than the second threshold value, or when the angle of the ankle joint of one foot in the swing phase is larger than the second threshold value.

In the present embodiment, the sarcopenia determination unit 113 may determine that the subject is a pre-sarcopenia subject when the angle of the knee joint of one leg in the swing phase is larger than the second threshold value. The sarcopenia determination unit 113 determines whether or not the mean value of time series data of the angle of the knee joint of one leg of the subject in a period of 61% to 100% of one walking cycle is larger than the second threshold value. The sarcopenia determination unit 113 determines that the subject is a pre-sarcopenia subject when the mean value of the time series data of the angle of the knee joint of the one leg of the subject in the period of 61% to 100% of one walking cycle is larger than the second threshold value. On the other hand, the sarcopenia determination unit 113 determines that the subject is not a pre-sarcopenia subject, i.e., the subject is a healthy subject when the mean value of the time series data of the angle of the knee joint of the one leg of the subject in the period of 61% to 100% of one walking cycle is equal to or less than the second threshold value.

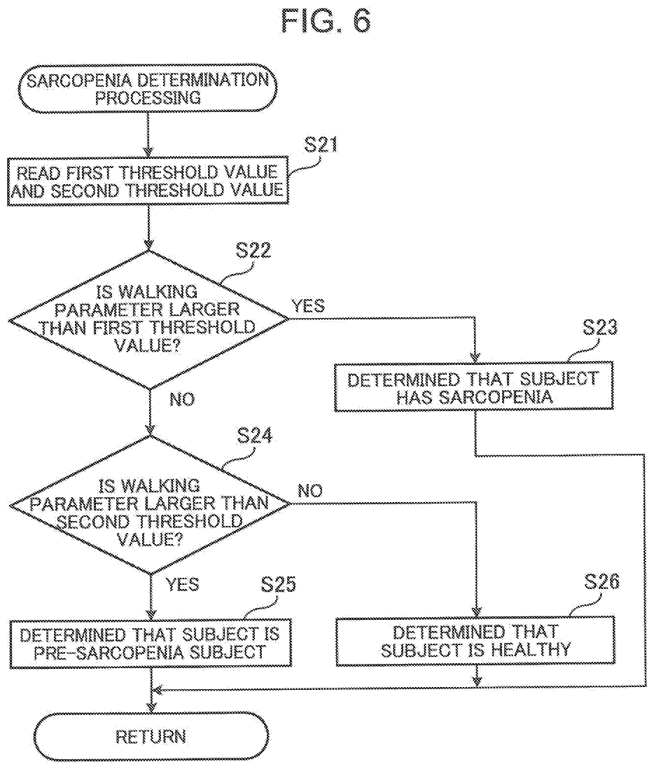

FIG. 6 is a flowchart for explaining another example of the sarcopenia determination processing in step S4 of FIG. 4.

First, in step S21, the sarcopenia determination unit 113 reads the first threshold value and the second threshold value from the memory 12.

Next, in step S22, the sarcopenia determination unit 113 determines whether or not the walking parameter detected by the walking parameter detection unit 112 is larger than the first threshold value. The walking parameter in the present embodiment is a mean value of the time series data of the angle of the knee joint of one leg of the subject in a period of 61% to 100% of one walking cycle. The sarcopenia determination unit 113 determines whether or not the mean value of time series data of the angle of the knee joint of one leg of the subject in a period of 61% to 100% of one walking cycle is larger than the first threshold value.

Here, when it is determined that the walking parameter is larger than the first threshold value (YES in step S22), the sarcopenia determination unit 113 determines in step S23 that the subject has sarcopenia.

On the other hand, when it is determined that the walking parameter is equal to or less than the first threshold value (NO in step S22), the sarcopenia determination unit 113 determines in step S24 whether or not the walking parameter detected by the walking parameter detection unit 112 is larger than the second threshold value. The walking parameter in the present embodiment is a mean value of the time series data of the angle of the knee joint of one leg of the subject in a period of 61% to 100% of one walking cycle. The sarcopenia determination unit 113 determines whether or not the mean value of time series data of the angle of the knee joint of one leg of the subject in a period of 61% to 100% of one walking cycle is larger than the second threshold value.

Here, when it is determined that the walking parameter is larger than the second threshold value (YES in step S24), the sarcopenia determination unit 113 determines in step S25 that the subject is a pre-sarcopenia subject.

On the other hand, when it is determined that the walking parameter is equal to or less than the second threshold value (NO in step S24), the sarcopenia determination unit 113 determines in step S26 that the subject is not a pre-sarcopenia subject, i.e., the subject is a healthy subject.

Thus, in the present embodiment, the angle of the knee joint of one leg in the swing phase of the walking subject is a parameter correlated with sarcopenia of the subject. Walking motion of subjects with sarcopenia tends to be different from walking motion of subjects without sarcopenia. Therefore, sarcopenia of the subject is determined by using a parameter correlated with sarcopenia of the walking subject, the sarcopenia of the subject can be evaluated with high accuracy.

Furthermore, the angle of the knee joint of one leg in the swing phase of a walking subject can be easily detected from image data obtained by capturing an image of the walking subject, for example, and hence a large-scale device is unnecessary. Therefore, the present configuration can easily evaluate sarcopenia of a subject.

The walking parameters and the prediction models in the present embodiment are decided by experiments. Hereinafter, a decision method of a walking parameter and a prediction model in the present embodiment will be described.

The total number of subjects who participated in the experiment was 65. All subjects were female. Conventionally, there are various sarcopenia determination criteria. The sarcopenia determination criterion employed this time was that the limb skeletal muscle mass is less than 5.8 (kg/m′) and the grip strength is less than 19.3 (kg), or the limb skeletal muscle mass is less than 5.8 (kg/m²) and the walking speed is equal to or less than 1.19 (m/s). The limb skeletal muscle mass has a value obtained by dividing a total muscle mass of both arms and both legs by the square of the body height. It was determined that the subject has sarcopenia when the limb skeletal muscle mass was less than 5.8 (kg/m²) and the grip strength was less than 19.3 (kg), or when the limb skeletal muscle mass was less than 5.8 (kg/m²) and the walking speed was equal to or less than 1.19 (m/s). It is to be noted that the criterion value used for the above determination is targeted at females, and in the case where the subject is male, it is determined that the subject has sarcopenia when the limb skeletal muscle mass is less than 7.0 (kg/m′) and the grip strength is less than 30.3 (kg), or when the limb skeletal muscle mass is less than 7.0 (kg/m′) and the walking speed is equal to or less than 1.27 (m/s).

In addition, it was determined that the subject is a pre-sarcopenia subject when only the limb skeletal muscle mass was less than the criterion value. That is, it was determined that the subject is a pre-sarcopenia subject when only the limb skeletal muscle mass was less than 5.8 (kg/m).

It is to be noted that the sarcopenia determination criterion is an example, and is not limited to the above.

As a result of the determination, of the subjects, nine had sarcopenia, 30 were pre-sarcopenia subjects, and 26 were healthy subjects. In the experiment, the subjects performed walking in front of the camera. Images of the walking subjects were captured by the camera, and the skeleton data of each subject was extracted from the moving image data. Then, time series data of the angle of the knee joint of one leg of each subject was detected from the extracted skeleton data.

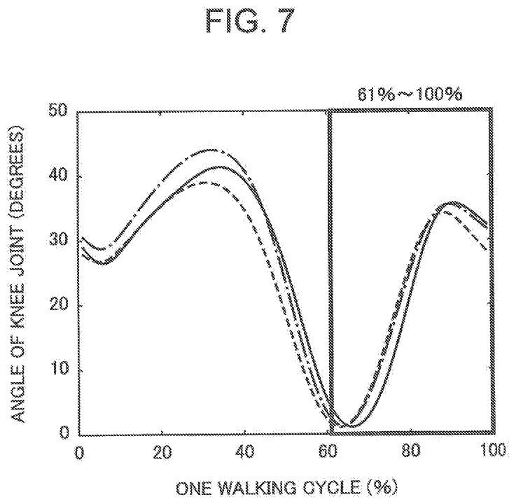

FIG. 7 is a view showing a change in the angle of the knee joint of one leg in one walking cycle in the present embodiment. In FIG. 7, the vertical axis represents the angle of the knee joint, and the horizontal axis represents one normalized walking cycle. In addition, in FIG. 7, the dashed line represents an average waveform of the angles of the knee joint of one leg of the healthy subjects, the dashed-dotted line represents an average waveform of the angles of the knee joint of one leg of the pre-sarcopenia subjects, and the solid line represents an average waveform of the angles of the knee joint of one leg of the sarcopenia subjects.

In the experiment, one normalized walking cycle was divided into ten intervals, and the mean value of the angles of the knee joint of one leg in one interval or two or more consecutive intervals was calculated for each subject. Then, a prediction model was created with which of a sarcopenia subject, a pre-sarcopenia subject, and a healthy subject the subject is as an objective variable, and with the mean value of the angles of the knee joint of one leg in a period of 61% to 100% of one walking cycle as an explanatory variable. The prediction model was evaluated by cross validation. Leave-one-out cross validation was adopted as the cross validation. Then, a receiver operating characteristic (ROC) curve of the prediction model in which a healthy subject and a sarcopenia subject were determined, and an ROC curve of the prediction model in which a healthy subject and a pre-sarcopenia subject were determined were calculated. Furthermore, an area under curve (AUC) value of each of the two ROC curves of the prediction models was calculated.

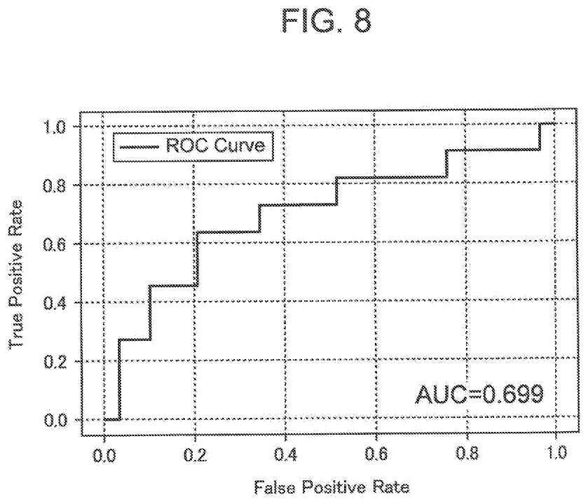

FIG. 8 is a view showing an ROC curve obtained as a result of determining a healthy subject and a sarcopenia subject using a prediction model in the present embodiment.

The prediction model in the present embodiment was created with which of a sarcopenia subject, a pre-sarcopenia subject, and a healthy subject the subject is as an objective variable, and with the mean value of the angles of the knee joint of one leg in a period of 61% to 100% of one walking cycle as an explanatory variable. In FIG. 8, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 8 was obtained by plotting the true positive rate and the false positive rate of the prediction model created with the mean value of the angles of the knee joint of one leg in a period of 61% to 100% of one walking cycle as an explanatory variable. The AUC value of the ROC curve shown in FIG. 8 was 0.699. The AUC value is the area below the ROC curve. It is true that the larger the AUC value is (the more it approaches 1), the higher the performance of the prediction model is.

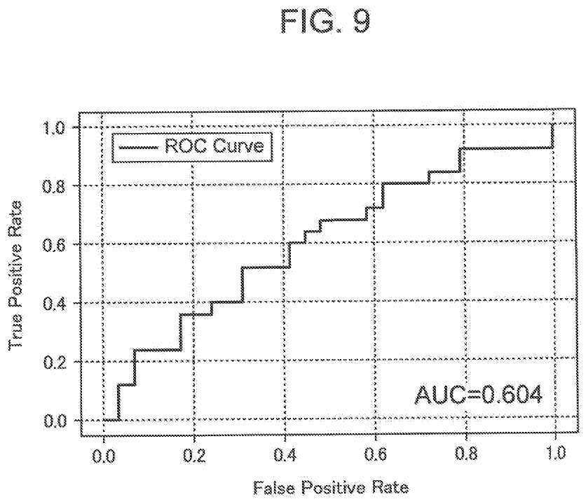

FIG. 9 is a view showing an ROC curve obtained as a result of determining a healthy subject and a pre-sarcopenia subject using the prediction model in the present embodiment. In FIG. 9, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 9 was obtained by plotting the true positive rate and the false positive rate of the prediction model created with the mean value of the angles of the knee joint of one leg in a period of 61% to 100% of one walking cycle as an explanatory variable. The AUC value of the ROC curve shown in FIG. 9 was 0.604.

In the present embodiment, the mean value of the angles of the knee joint of one leg in a period of 61% to 100% of one walking cycle is determined as a walking parameter. In addition, the prediction model created with the mean value of the angles of the knee joint of one leg in a period of 61% to 100% of one walking cycle as an explanatory variable is determined as a prediction model to be used by the sarcopenia determination unit 113.

The memory 12 stores in advance a prediction model generated with the mean value of time series data of the angle of the knee joint of one leg in a period of 61% to 100% of one walking cycle as an input value and with which of a sarcopenia subject, a pre-sarcopenia subject, and a healthy subject the subject is as an output value. The walking parameter detection unit 112 detects time series data of the angle of the knee joint of one leg in a period of 61% to 100% of one walking cycle. By inputting the mean value of time series data of the angle of the knee joint of one leg in a period of 61% to 100% of one walking cycle to the prediction model, the sarcopenia determination unit 113 acquires, from the prediction model, a determination result indicating which of a sarcopenia subject, a pre-sarcopenia subject, or a healthy subject the subject is.

In addition, in the period of 61% to 100% of one walking cycle shown in FIG. 7, the average waveform of the angle of the knee joint of one leg of the sarcopenia subjects is larger than the average waveform of the angle of the knee joint of one leg of the pre-sarcopenia subjects. Therefore, a value between an average of the mean values of time series data of the angle of the knee joint of one leg in a period of 61% to 100% of one walking cycle of the sarcopenia subjects and an average of the mean values of time series data of the angle of the knee joint of one leg in a period of 61% to 100% of one walking cycle of the pre-sarcopenia subjects, having been experimentally obtained, may be stored in the memory 12 as the first threshold value. The sarcopenia determination unit 113 may determine whether or not the subject has sarcopenia by comparing the mean value of time series data of the angle of the knee joint of one leg of the subject in a period of 61% to 100% of one walking cycle with the first threshold value stored in advance.

In addition, in the period of 61% to 100% of one walking cycle shown in FIG. 7, the average waveform of the angle of the knee joint of one leg of the pre-sarcopenia subjects is larger than the average waveform of the angle of the knee joint of one leg of the healthy subjects. Therefore, a value between an average of the mean values of time series data of the angle of the knee joint of one leg in a period of 61% to 100% of one walking cycle of the pre-sarcopenia subjects and an average of the mean values of time series data of the angle of the knee joint of one leg in a period of 61% to 100% of one walking cycle of the healthy subjects, having been experimentally obtained, may be stored in the memory 12 as the second threshold value. The sarcopenia determination unit 113 may determine whether or not the subject is a pre-sarcopenia subject by comparing the mean value of time series data of the angle of the knee joint of one leg of the subject in a period of 61% to 100% of one walking cycle with the second threshold value stored in advance.

It is to be noted that while in the present embodiment, the walking parameter is a mean value of time series data of the angle of the knee joint of one leg in a period of 61% to 100% of one walking cycle, the present disclosure is not particularly limited thereto. Various examples of the walking parameters of the present embodiment will be described below.

First, the walking parameters in the first modification of the present embodiment will be described.

The walking parameter in the first modification of the present embodiment may be a mean value of time series data of the angle of the knee joint of one leg in a predetermined period of the stance phase of one leg of the subject.

In the first modification of the present embodiment, similar to the above experiment, time series data of the angle of one knee joint of each of the plurality of subjects was detected from the skeleton data of a plurality of subjects including a sarcopenia subject, a pre-sarcopenia subject, and a healthy subject. In addition, a prediction model was created with which of a sarcopenia subject, a pre-sarcopenia subject, and a healthy subject the subject is as an objective variable, and with the mean value of the time series data of the angle of one knee joint in a predetermined period of the stance phase as an explanatory variable. The predetermined period is a period of 1% to 60% of one walking cycle. The prediction model was evaluated by cross validation. Leave-one-out cross validation was adopted as the cross validation. Then, the ROC curve of the prediction model was calculated. Furthermore, the AUC value of the ROC curve of the prediction model was calculated.

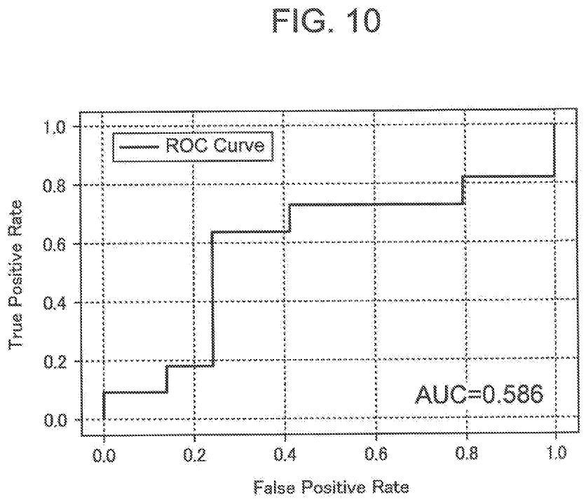

FIG. 10 is a view showing an ROC curve obtained as a result of determining a healthy subject and a sarcopenia subject using a prediction model in the first modification of the present embodiment.

The prediction model in the first modification of the present embodiment was created with which of a sarcopenia subject, a pre-sarcopenia subject, and a healthy subject the subject is as an objective variable, and with the mean value of the angles of the knee joint of one leg in a period of 1% to 60% of one walking cycle as an explanatory variable. In FIG. 10, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 10 was obtained by plotting the true positive rate and the false positive rate of the prediction model created with the mean value of the angles of the knee joint of one leg in a period of 1% to 60% of one walking cycle as an explanatory variable. The AUC value of the ROC curve shown in FIG. 10 was 0.586.

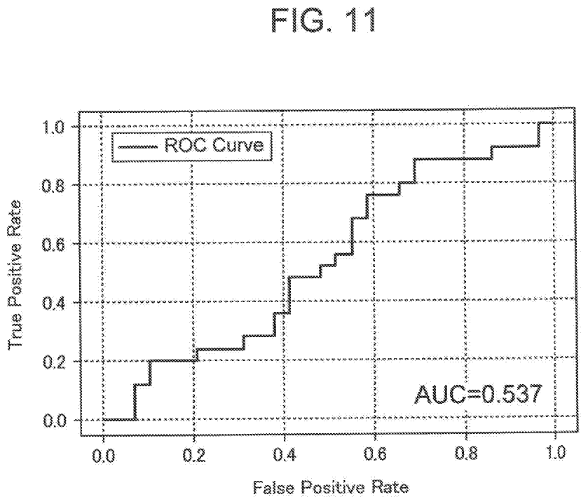

FIG. 11 is a view showing an ROC curve obtained as a result of determining a healthy subject and a pre-sarcopenia subject using the prediction model in the first modification of the present embodiment.

In FIG. 11, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 11 was obtained by plotting the true positive rate and the false positive rate of the prediction model created with the mean value of the angles of the knee joint of one leg in a period of 1% to 60% of one walking cycle as an explanatory variable. The AUC value of the ROC curve shown in FIG. 11 was 0.537.

In the first modification of the present embodiment, the mean value of the angles of the knee joint of one leg in a period of 1% to 60% of one walking cycle is determined as a walking parameter. In addition, the prediction model created with the mean value of the angles of the knee joint of one leg in a period of 1% to 60% of one walking cycle as an explanatory variable is determined as a prediction model to be used by the sarcopenia determination unit 113.

The walking parameter detection unit 112 detects, from walking data, the angle of the knee joint of one leg in a predetermined period of the stance phase of one leg of the subject. The walking parameter detection unit 112 detects the angle of the knee joint of one leg in a predetermined period of the stance phase from the time series skeleton data corresponding to the one walking cycle having been clipped. In particular, the walking parameter detection unit 112 detects time series data of the angle of the knee joint in a predetermined period of the stance phase of one leg. More specifically, the predetermined period is a period of 1% to 60% of one walking cycle. The walking parameter detection unit 112 detects time series data of the angle of the knee joint of one leg in a period of 1% to 60% of one walking cycle. In addition, the walking parameter detection unit 112 calculates the mean value of time series data of the angle of the knee joint of one leg in a period of 1% to 60% of one walking cycle.

The memory 12 stores in advance a prediction model generated with the angle of the knee joint of one leg in a period of 1% to 60% of one walking cycle as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the time series data of the angle of the knee joint of one leg in a period of 1% to 60% of one walking cycle as an explanatory variable. In particular, the memory 12 stores in advance a prediction model generated with the mean value of time series data of the angle of the knee joint of one leg in a period of 1% to 60% of one walking cycle as an input value and with which of a sarcopenia subject, a pre-sarcopenia subject, and a healthy subject the subject is as an output value.

The sarcopenia determination unit 113 determines whether or not the subject has sarcopenia using the mean value of time series data of the angle of the knee joint of one leg of a predetermined period of the stance phase. The predetermined period is a period of 1% to 60% of one walking cycle. The sarcopenia determination unit 113 determines whether or not the subject has sarcopenia by inputting the mean value of time series data of the angle of the knee joint of one leg of a predetermined period of the stance phase detected by the walking parameter detection unit 112 to a prediction model generated with the mean value of time series data of the angle of the knee joint of one leg of a predetermined period of the stance phase as an input value and with whether or not the subject has sarcopenia as an output value.

In addition, the sarcopenia determination unit 113 determines which of a sarcopenia subject, a pre-sarcopenia subject, and a healthy subject the subject is by using the mean value of time series data of the angle of the knee joint in a predetermined period of the stance phase of one leg. The predetermined period is a period of 1% to 60% of one walking cycle. More specifically, the sarcopenia determination unit 113 determines which of a sarcopenia subject, a pre-sarcopenia subject, and a healthy subject the subject is by using the mean value of time series data of the angle of the knee joint of one leg in a period of 1% to 60% of one walking cycle. By inputting the mean value of time series data of the angle of one knee joint in a period of 1% to 60% of one walking cycle to the prediction model, the sarcopenia determination unit 113 acquires, from the prediction model, a determination result indicating which of a sarcopenia subject, a pre-sarcopenia subject, or a healthy subject the subject is.

Subsequently, the walking parameters in the second modification of the present embodiment will be described.

The walking parameter in the second modification of the present embodiment may be a mean value of time series data of the angle of the knee joint of one leg in a period of 50% to 60% of one walking cycle.

In the second modification of the present embodiment, similar to the above experiment, time series data of the angle of the knee joint of one leg of each of the plurality of subjects was detected. In addition, a prediction model was created with which of a sarcopenia subject, a pre-sarcopenia subject, and a healthy subject the subject is as an objective variable, and with the mean value of the angles of the knee joint of one leg in a period of 50% to 60% of one walking cycle as an explanatory variable. The prediction model was evaluated by cross validation. Leave-one-out cross validation was adopted as the cross validation. Then, the ROC curve of the prediction model was calculated. Furthermore, the AUC value of the ROC curve of the prediction model was calculated.

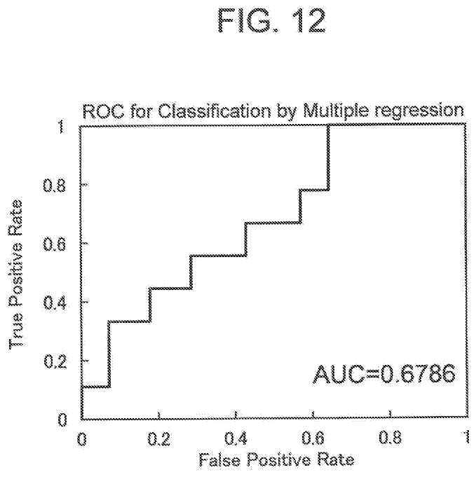

FIG. 12 is a view showing an ROC curve obtained as a result of determining a healthy subject and a sarcopenia subject using a prediction model in the second modification of the present embodiment.

The prediction model in the second modification of the present embodiment was created with which of a sarcopenia subject, a pre-sarcopenia subject, and a healthy subject the subject is as an objective variable, and with the mean value of the angles of one knee joint in a period of 50% to 60% of one walking cycle as an explanatory variable. In FIG. 12, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 12 was obtained by plotting the true positive rate and the false positive rate of the prediction model created with the mean value of the angles of the knee joint of one leg in a period of 50% to 60% of one walking cycle as an explanatory variable. The AUC value of the ROC curve shown in FIG. 12 was 0.6786.

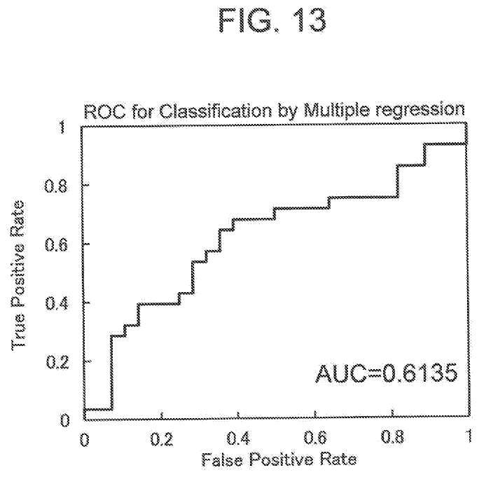

FIG. 13 is a view showing an ROC curve obtained as a result of determining a healthy subject and a pre-sarcopenia subject using the prediction model in the second modification of the present embodiment.

In FIG. 13, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 13 was obtained by plotting the true positive rate and the false positive rate of the prediction model created with the mean value of the angles of the knee joint of one leg in a period of 50% to 60% of one walking cycle as an explanatory variable. The AUC value of the ROC curve shown in FIG. 13 was 0.6135.

In the second modification of the present embodiment, the mean value of the angles of the knee joint of one leg in a period of 50% to 60% of one walking cycle is determined as a walking parameter. In addition, the prediction model created with the mean value of the angles of the knee joint of one leg in a period of 50% to 60% of one walking cycle as an explanatory variable is determined as a prediction model to be used by the sarcopenia determination unit 113.

The walking parameter detection unit 112 detects time series data of the angle of the knee joint in a predetermined period of the stance phase of one leg. More specifically, the predetermined period is a period of 50% to 60% of one walking cycle. The walking parameter detection unit 112 detects time series data of the angle of the knee joint of one leg in a period of 50% to 60% of one walking cycle. In addition, the walking parameter detection unit 112 calculates the mean value of time series data of the angle of the knee joint of one leg in a period of 50% to 60% of one walking cycle.

The memory 12 stores in advance a prediction model generated with the angle of the knee joint of one leg in a period of 50% to 60% of one walking cycle as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the time series data of the angle of the knee joint of one leg in a period of 50% to 60% of one walking cycle as an explanatory variable. In particular, the memory 12 stores in advance a prediction model generated with the mean value of time series data of the angle of the knee joint of one leg in a period of 50% to 60% of one walking cycle as an input value and with which of a sarcopenia subject, a pre-sarcopenia subject, and a healthy subject the subject is as an output value.

The sarcopenia determination unit 113 determines whether or not the subject has sarcopenia using the mean value of time series data of the angle of the knee joint of one leg in a predetermined period of the stance phase. The predetermined period is a period of 50% to 60% of one walking cycle. The sarcopenia determination unit 113 determines whether or not the subject has sarcopenia by inputting the mean value of time series data of the angle of the knee joint of one leg in a predetermined period of the stance phase detected by the walking parameter detection unit 112 to a prediction model generated with the mean value of time series data of the angle of the knee joint of one leg in a predetermined period of the stance phase as an input value and with whether or not the subject has sarcopenia as an output value.

In addition, the sarcopenia determination unit 113 determines which of a sarcopenia subject, a pre-sarcopenia subject, and a healthy subject the subject is by using the mean value of time series data of the angle of the knee joint in a predetermined period of the stance phase of one leg. The predetermined period is a period of 50% to 60% of one walking cycle. More specifically, the sarcopenia determination unit 113 determines which of a sarcopenia subject, a pre-sarcopenia subject, and a healthy subject the subject is by using the mean value of time series data of the angle of the knee joint of one leg in a period of 50% to 60% of one walking cycle. By inputting the mean value of time series data of the angle of one knee joint in a period of 50% to 60% of one walking cycle to the prediction model, the sarcopenia determination unit 113 acquires, from the prediction model, a determination result indicating which of a sarcopenia subject, a pre-sarcopenia subject, or a healthy subject the subject is.

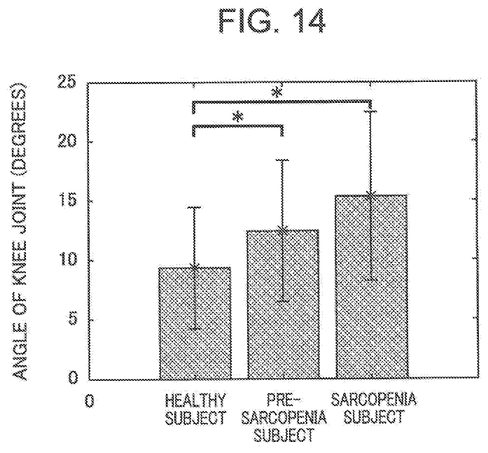

FIG. 14 is a view showing an average of mean values of time series data of the angle of the knee joint of one leg of sarcopenia subjects, an average of mean values of time series data of the angle of the knee joint of one leg of pre-sarcopenia subjects, and an average of mean values of time series data of the angle of the knee joint of one leg of healthy subjects, in the second modification of the present embodiment.

As shown in FIG. 14, an average of the mean values of time series data of the angle of the knee joint of one leg of the sarcopenia subjects in a period of 50% to 60% of one walking cycle was 15.3 degrees, an average of the mean values of time series data of the angle of the knee joint of one leg of the pre-sarcopenia subjects in a period of 50% to 60% of one walking cycle was 12.4 degrees, and an average of the mean values of time series data of the angle of the knee joint of one leg of the healthy subjects in a period of 50% to 60% of one walking cycle was 9.3 degrees.

Thus, in the period of 50% to 60% of one walking cycle, the average of the mean values of time series data of the angle of the knee joint of one leg of the sarcopenia subjects is larger than the average of the mean values of time series data of the angle of the knee joint of one leg of the pre-sarcopenia subjects. Therefore, a value between an average of the mean values of time series data of the angle of the knee joint of one leg in a period of 50% to 60% of one walking cycle of the sarcopenia subjects and an average of the mean values of time series data of the angle of the knee joint of one leg in a period of 50% to 60% of one walking cycle of the pre-sarcopenia subjects, having been experimentally obtained, may be stored in the memory 12 as the first threshold value. The sarcopenia determination unit 113 may determine whether or not the subject has sarcopenia by comparing the mean value of time series data of the angle of the knee joint of one leg of the subject in a period of 50% to 60% of one walking cycle with the first threshold value stored in advance.

In addition, in the period of 50% to 60% of one walking cycle, the average of the mean values of time series data of the angle of the knee joint of one leg of the pre-sarcopenia subjects is larger than the average of the mean values of time series data of the angle of the knee joint of one leg of the healthy subjects. Therefore, a value between an average of the mean values of time series data of the angle of the knee joint of one leg in a period of 50% to 60% of one walking cycle of the pre-sarcopenia subjects and an average of the mean values of time series data of the angle of the knee joint of one leg in a period of 50% to 60% of one walking cycle of the healthy subjects, having been experimentally obtained, may be stored in the memory 12 as the second threshold value. The sarcopenia determination unit 113 may determine whether or not the subject is a pre-sarcopenia subject by comparing the mean value of time series data of the angle of the knee joint of one leg of the subject in a period of 50% to 60% of one walking cycle with the second threshold value stored in advance.

Subsequently, the walking parameters in the third modification of the present embodiment will be described.

The walking parameter in the third modification of the present embodiment may be a mean value of time series data of the vertical displacement of the toe of one foot in a predetermined period of the stance phase of one leg.

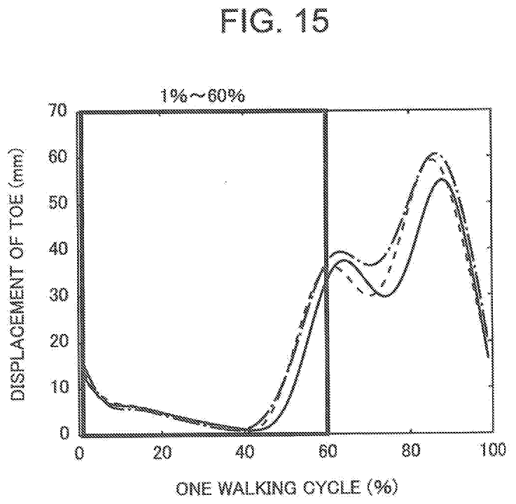

FIG. 15 is a view showing a vertical displacement of the toe of one foot in one walking cycle in a third modification of the present embodiment. In FIG. 15, the vertical axis represents the vertical displacement of the toe, and the horizontal axis represents one normalized walking cycle. In addition, in FIG. 15, the dashed line represents an average waveform of the vertical displacements of the toe of one foot of the healthy subjects, the dashed-dotted line represents an average waveform of the vertical displacements of the toe of the pre-sarcopenia subjects, and the solid line represents an average waveform of the vertical displacements of the toe of the sarcopenia subjects.

In the third modification of the present embodiment, similar to the above experiment, time series data of the vertical displacement of the toe of one foot of each of the plurality of subjects was detected from the skeleton data of a plurality of subjects including a sarcopenia subject, a pre-sarcopenia subject, and a healthy subject. As shown in FIG. 2, a vertical displacement β of the toe is a vertical displacement of the feature point 213 indicating the toe.

In the experiment, one normalized walking cycle was divided into ten intervals, and the mean value of the vertical displacements of the toe of one foot in one interval or two or more consecutive intervals was calculated for each subject. Then, a prediction model was created with which of a sarcopenia subject, a pre-sarcopenia subject, and a healthy subject the subject is as an objective variable, and with the mean value of the vertical displacements of the toe of one foot in a period of 1% to 60% of one walking cycle as an explanatory variable. The prediction model was evaluated by cross validation. Leave-one-out cross validation was adopted as the cross validation. Then, an ROC curve of the prediction model in which a healthy subject and a sarcopenia subject were determined, and an ROC curve of the prediction model in which a healthy subject and a pre-sarcopenia subject were determined were calculated. Furthermore, the AUC value of each of the two ROC curves of the prediction model was calculated.

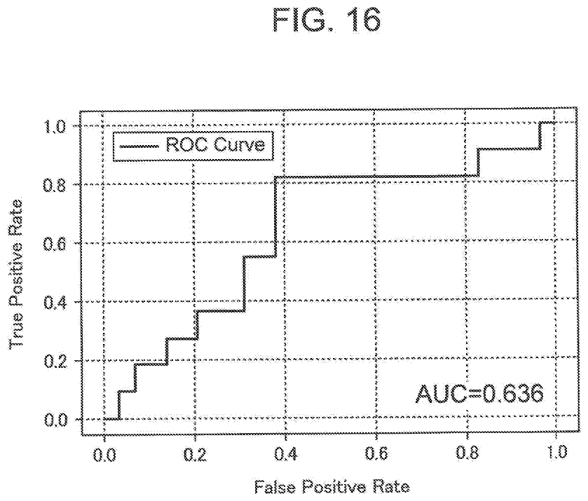

FIG. 16 is a view showing an ROC curve obtained as a result of determining a healthy subject and a sarcopenia subject using a prediction model in the third modification of the present embodiment.

The prediction model in the third modification of the present embodiment was created with which of a sarcopenia subject, a pre-sarcopenia subject, and a healthy subject the subject is as an objective variable, and with the mean value of the vertical displacements of the toe of one foot in a period of 1% to 60% of one walking cycle as an explanatory variable. In FIG. 16, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 16 was obtained by plotting the true positive rate and the false positive rate of the prediction model created with the mean value of the vertical displacements of the toe of one foot in a period of 1% to 60% of one walking cycle as an explanatory variable. The AUC value of the ROC curve shown in FIG. 16 was 0.636.

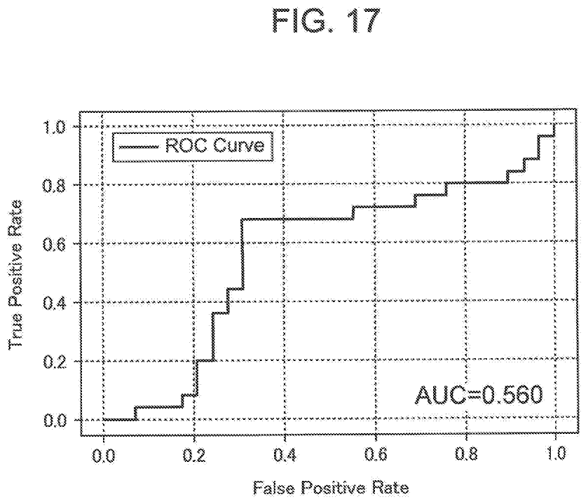

FIG. 17 is a view showing an ROC curve obtained as a result of determining a healthy subject and a pre-sarcopenia subject using the prediction model in the third modification of the present embodiment.

In FIG. 17, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 17 was obtained by plotting the true positive rate and the false positive rate of the prediction model created with the mean value of the vertical displacements of the toe of one foot in a period of 1% to 60% of one walking cycle as an explanatory variable. The AUC value of the ROC curve shown in FIG. 17 was 0.560.

In the third modification of the present embodiment, the mean value of the vertical displacements of the toe of one foot in a period of 1% to 60% of one walking cycle is determined as a walking parameter. In addition, the prediction model created with the mean value of the vertical displacements of the toe of one foot in a period of 1% to 60% of one walking cycle as an explanatory variable is determined as a prediction model to be used by the sarcopenia determination unit 113.

The walking parameter detection unit 112 detects, from walking data, the vertical displacement of the toe of one foot of the subject. The walking parameter detection unit 112 detects the vertical displacement of the toe of one foot of the subject from the time series skeleton data corresponding to the one walking cycle having been clipped. In particular, the walking parameter detection unit 112 detects time series data of the vertical displacement of the toe of one foot in a predetermined period of the stance phase of one leg. More specifically, the predetermined period is a period of 1% to 60% of one walking cycle. The walking parameter detection unit 112 detects time series data of the vertical displacement of the toe of one foot in a period of 1% to 60% of one walking cycle. In addition, the walking parameter detection unit 112 calculates the mean value of time series data of the vertical displacement of the toe of one foot in a period of 1% to 60% of one walking cycle.

It is to be noted that in the third modification of the present embodiment, since the one walking cycle is a period from when the right foot of the subject touches the ground to when the right foot touches the ground again, the walking parameter detection unit 112 detects the vertical displacement β of the toe of the right foot in the stance phase of the right leg. In a case where one walking cycle is a period from when the left foot of the subject touches the ground to when the left foot touches the ground again, the walking parameter detection unit 112 may detect the vertical displacement β of the toe of the left foot when the left foot is in the stance phase.

The memory 12 stores in advance a prediction model generated with the vertical displacement of the toe of one foot in a period of 1% to 60% of one walking cycle as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the time series data of the vertical displacement of the toe of one foot in a period of 1% to 60% of one walking cycle an explanatory variable. In particular, the memory 12 stores in advance a prediction model generated with the mean value of time series data of the vertical displacement of the toe of one foot in a period of 1% to 60% of one walking cycle as an input value and with which of a sarcopenia subject, a pre-sarcopenia subject, and a healthy subject the subject is as an output value.

The sarcopenia determination unit 113 determines whether or not the subject has sarcopenia using the vertical displacement of the toe of one foot. The sarcopenia determination unit 113 determines whether or not the subject has sarcopenia by inputting the vertical displacement of the toe of one foot detected by the walking parameter detection unit 112 to a prediction model generated with the vertical displacement of the toe of one foot as an input value and with whether or not the subject has sarcopenia as an output value.

In addition, the sarcopenia determination unit 113 determines whether or not the subject has sarcopenia using the mean value of time series data of the vertical displacement of the toe of one foot in a predetermined period of the stance phase of one leg. The predetermined period is a period of 1% to 60% of one walking cycle. The sarcopenia determination unit 113 determines whether or not the subject has sarcopenia using the mean value of time series data of the vertical displacement of the toe of one foot in a period of 1% to 60% of one walking cycle. By inputting the mean value of time series data of the vertical displacement of the toe of one foot in a period of 1% to 60% of one walking cycle to the prediction model, the sarcopenia determination unit 113 acquires, from the prediction model, a determination result indicating whether or not the subject has sarcopenia.

In addition, the sarcopenia determination unit 113 determines which of a sarcopenia subject, a pre-sarcopenia subject, and a healthy subject the subject is by using the mean value of time series data of the vertical displacement of the toe of one foot in a predetermined period of the stance phase of one leg. The predetermined period is a period of 1% to 60% of one walking cycle. More specifically, the sarcopenia determination unit 113 determines which of a sarcopenia subject, a pre-sarcopenia subject, and a healthy subject the subject is by using the mean value of time series data of the vertical displacement of the toe of one foot in a period of 1% to 60% of one walking cycle. By inputting the mean value of time series data of the vertical displacement of the toe of one foot in a period of 1% to 60% of one walking cycle to the prediction model, the sarcopenia determination unit 113 acquires, from the prediction model, a determination result indicating which of a sarcopenia subject, a pre-sarcopenia subject, or a healthy subject the subject is.

Subsequently, the walking parameters in the fourth modification of the present embodiment will be described.

The walking parameter in the fourth modification of the present embodiment may be a mean value of time series data of the vertical displacement of the toe of one foot in a predetermined period of the swing phase of one leg of the subject.

In the fourth modification of the present embodiment, similar to the above experiment, time series data of the vertical displacement of the toe of one foot of each of the plurality of subjects was detected from the skeleton data of a plurality of subjects including a sarcopenia subject, a pre-sarcopenia subject, and a healthy subject. In addition, a prediction model was created with which of a sarcopenia subject, a pre-sarcopenia subject, and a healthy subject the subject is as an objective variable, and with the mean value of the time series data of the angle of one knee joint in a predetermined period of the swing phase as an explanatory variable. The predetermined period is a period of 61% to 100% of one walking cycle. The prediction model was evaluated by cross validation. Leave-one-out cross validation was adopted as the cross validation. Then, the ROC curve of the prediction model was calculated. Furthermore, the AUC value of the ROC curve of the prediction model was calculated.

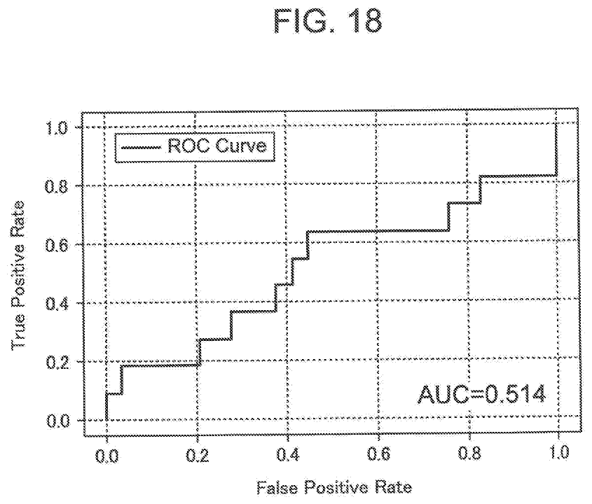

FIG. 18 is a view showing an ROC curve obtained as a result of determining a healthy subject and a sarcopenia subject using a prediction model in the fourth modification of the present embodiment.

The prediction model in the fourth modification of the present embodiment was created with which of a sarcopenia subject, a pre-sarcopenia subject, and a healthy subject the subject is as an objective variable, and with the mean value of the vertical displacements of the toe of one foot in a period of 61% to 100% of one walking cycle as an explanatory variable. In FIG. 18, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 18 was obtained by plotting the true positive rate and the false positive rate of the prediction model created with the mean value of the vertical displacements of the toe of one foot in a period of 61% to 100% of one walking cycle as an explanatory variable. The AUC value of the ROC curve shown in FIG. 18 was 0.514.

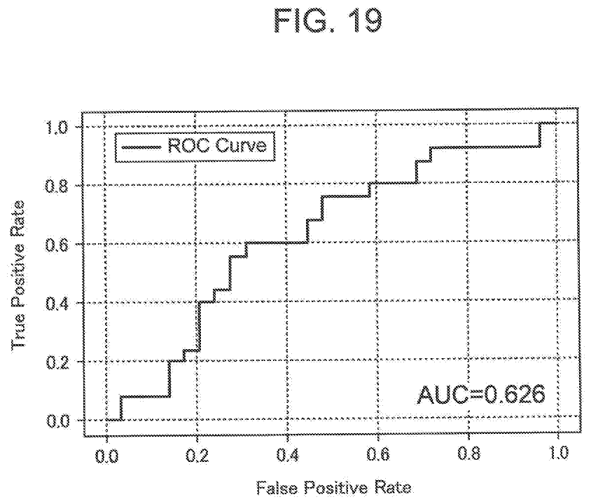

FIG. 19 is a view showing an ROC curve obtained as a result of determining a healthy subject and a pre-sarcopenia subject using the prediction model in the fourth modification of the present embodiment.

In FIG. 19, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 19 was obtained by plotting the true positive rate and the false positive rate of the prediction model created with the mean value of the vertical displacements of the toe of one foot in a period of 61% to 100% of one walking cycle as an explanatory variable. The AUC value of the ROC curve shown in FIG. 19 was 0.626.

In the fourth modification of the present embodiment, the mean value of the vertical displacements of the toe of one foot in a period of 61% to 100% of one walking cycle is determined as a walking parameter. In addition, the prediction model created with the mean value of the vertical displacements of the toe of one foot in a period of 61% to 100% of one walking cycle as an explanatory variable is determined as a prediction model to be used by the sarcopenia determination unit 113.

The walking parameter detection unit 112 detects, from walking data, the vertical displacement of the toe of one foot in a predetermined period of the swing phase of one leg of the subject. The walking parameter detection unit 112 detects the vertical displacement of the toe of one foot in a predetermined period of the swing phase from the time series skeleton data corresponding to the one walking cycle having been clipped. In particular, the walking parameter detection unit 112 detects time series data of the vertical displacement of the toe of one foot in a predetermined period of the swing phase of one leg. More specifically, the predetermined period is a period of 61% to 100% of one walking cycle. The walking parameter detection unit 112 detects time series data of the vertical displacement of the toe of one foot in a period of 61% to 100% of one walking cycle. In addition, the walking parameter detection unit 112 calculates the mean value of time series data of the vertical displacement of the toe of one foot in a period of 61% to 100% of one walking cycle.

The memory 12 stores in advance a prediction model generated with the vertical displacement of the toe of one foot in a period of 61% to 100% of one walking cycle as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the time series data of the vertical displacement of the toe of one foot in a period of 61% to 100% of one walking cycle an explanatory variable. In particular, the memory 12 stores in advance a prediction model generated with the mean value of time series data of the vertical displacement of the toe of one foot in a period of 61% to 100% of one walking cycle as an input value and with which of a sarcopenia subject, a pre-sarcopenia subject, and a healthy subject the subject is as an output value.

The sarcopenia determination unit 113 determines whether or not the subject has sarcopenia using the mean value of time series data of the vertical displacement of the toe of one foot in a predetermined period of the swing phase. The predetermined period is a period of 61% to 100% of one walking cycle. The sarcopenia determination unit 113 determines whether or not the subject has sarcopenia by inputting the mean value of time series data of the vertical displacement of the toe of one foot in a predetermined period of the swing phase detected by the walking parameter detection unit 112 to a prediction model generated with the mean value of time series data of the vertical displacement of the toe of one foot in a predetermined period of the swing phase as an input value and with whether or not the subject has sarcopenia as an output value.

In addition, the sarcopenia determination unit 113 determines which of a sarcopenia subject, a pre-sarcopenia subject, and a healthy subject the subject is by using the mean value of time series data of the vertical displacement of the toe of one foot in a predetermined period of the swing phase of one leg. The predetermined period is a period of 61% to 100% of one walking cycle. More specifically, the sarcopenia determination unit 113 determines which of a sarcopenia subject, a pre-sarcopenia subject, and a healthy subject the subject is by using the mean value of time series data of the vertical displacement of the toe of one foot in a period of 61% to 100% of one walking cycle. By inputting the mean value of time series data of the vertical displacement of the toe of one foot in a period of 61% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Subsequently, the walking parameters in the fifth modification of the present embodiment will be described.

The walking parameter in the fifth modification of the present embodiment may be a mean value of time series data of the vertical displacement of the toe of one foot in a period of 65% to 70% of one walking cycle.

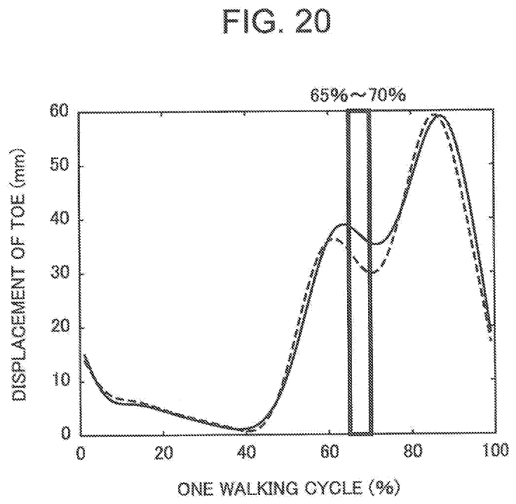

FIG. 20 is a view showing a vertical displacement of the toe of one foot in one walking cycle in a fifth modification of the present embodiment. In FIG. 20, the vertical axis represents the vertical displacement of the toe, and the horizontal axis represents one normalized walking cycle. In addition, in FIG. 20, the dashed line represents an average waveform of the vertical displacements of the toe of one foot of the healthy subjects, and the solid line represents an average waveform of the vertical displacements of the toe of one foot of the sarcopenia subjects or the pre-sarcopenia subjects.

It is to be noted that in the fourth modification of the present embodiment, it is determined which of a sarcopenia subject, a pre-sarcopenia subject, and a healthy subject the subject is, but in the fifth modification of the present embodiment, it is determined whether or not the subject is a sarcopenia subject or a pre-sarcopenia subject. It is to be noted that a subject who is neither a sarcopenia subject nor a pre-sarcopenia subject is determined to be a healthy subject.

In the fifth modification of the present embodiment, similar to the above experiment, time series data of the vertical displacement of the toe of one foot of each of the plurality of subjects was detected. In addition, a prediction model was created with whether or not the subject is a sarcopenia subject or a pre-sarcopenia subject as an objective variable, and with the mean value of the vertical displacements of the toe of one foot in a period of 65% to 70% of one walking cycle as an explanatory variable. The prediction model was evaluated by cross validation. Leave-one-out cross validation was adopted as the cross validation. Then, the ROC curve of the prediction model in which whether or not to be a sarcopenia subject or a pre-sarcopenia subject was determined was calculated. Furthermore, the AUC value of the ROC curve of the prediction model was calculated.

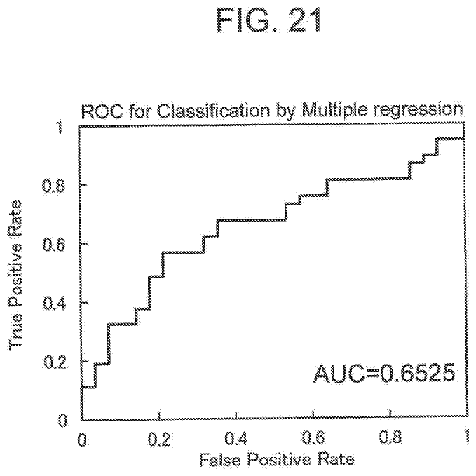

FIG. 21 is a view showing an ROC curve obtained as a result of determining whether or not to be a sarcopenia subject or a pre-sarcopenia subject using a prediction model in the fifth modification of the present embodiment.

The prediction model in the fifth modification of the present embodiment was created with whether or not the subject is a sarcopenia subject or a pre-sarcopenia subject as an objective variable, and with the mean value of the vertical displacements of the toe of one foot in a period of 65% to 70% of one walking cycle as an explanatory variable. In FIG. 21, the vertical axis represents the true positive rate, and the horizontal axis represents the false positive rate. The true positive rate indicates a ratio at which the prediction model has correctly determined a sarcopenia subject or a pre-sarcopenia subject as a sarcopenia subject or a pre-sarcopenia subject, and the false positive rate indicates a ratio at which the prediction model has incorrectly determined a healthy subject as a sarcopenia subject or a pre-sarcopenia subject.

The ROC curve shown in FIG. 21 was obtained by plotting the true positive rate and the false positive rate of the prediction model created with the mean value of the vertical displacements of the toe of one foot in a period of 65% to 70% of one walking cycle as an explanatory variable. The AUC value of the ROC curve shown in FIG. 21 was 0.6525.

In the fifth modification of the present embodiment, the mean value of the vertical displacements of the toe of one foot in a period of 65% to 70% of one walking cycle is determined as a walking parameter. In addition, the prediction model created with the mean value of the vertical displacements of the toe of one foot in a period of 65% to 70% of one walking cycle as an explanatory variable is determined as a prediction model to be used by the sarcopenia determination unit 113.

The walking parameter detection unit 112 detects time series data of the vertical displacement of the toe of one foot in a predetermined period of the swing phase of one leg. More specifically, the predetermined period is a period of 65% to 70% of one walking cycle. The walking parameter detection unit 112 detects time series data of the vertical displacement of the toe of one foot in a period of 65% to 70% of one walking cycle. In addition, the walking parameter detection unit 112 calculates the mean value of time series data of the vertical displacement of the toe of one foot in a period of 65% to 70% of one walking cycle.

The memory 12 stores in advance a prediction model generated with the vertical displacement of the toe of one foot in a period of 65% to 70% of one walking cycle as an input value and with whether or not the subject is a sarcopenia subject or a pre-sarcopenia subject as an output value. The prediction model is a regression model with whether or not the subject is a sarcopenia subject or a pre-sarcopenia subject as an objective variable, and with the time series data of the vertical displacement of the toe of one foot in a period of 65% to 70% of one walking cycle as an explanatory variable. In particular, the memory 12 stores in advance a prediction model generated with the mean value of time series data of the vertical displacement of the toe of one foot in a period of 65% to 70% of one walking cycle as an input value and with whether or not the subject is a sarcopenia subject or a pre-sarcopenia subject as an output value.

The sarcopenia determination unit 113 determines whether or not the subject has sarcopenia using the mean value of time series data of the vertical displacement of the toe of one foot of a predetermined period of the swing phase. The predetermined period is a period of 65% to 70% of one walking cycle. The sarcopenia determination unit 113 determines whether or not the subject has sarcopenia by inputting the mean value of time series data of the vertical displacement of the toe of one foot of a predetermined period of the swing phase detected by the walking parameter detection unit 112 to a prediction model generated with the mean value of time series data of the vertical displacement of the toe of one foot of a predetermined period of the swing phase as an input value and with whether or not the subject has sarcopenia as an output value.

In addition, the sarcopenia determination unit 113 determines whether or not the subject is a sarcopenia subject or a pre-sarcopenia subject by using the mean value of time series data of the vertical displacement of the toe of one foot in a predetermined period of the swing phase of one leg. The predetermined period is a period of 65% to 70% of one walking cycle. More specifically, the sarcopenia determination unit 113 determines whether or not the subject is a sarcopenia subject or a pre-sarcopenia subject by using the mean value of time series data of the vertical displacement of the toe of one foot in a period of 65% to 70% of one walking cycle. By inputting the mean value of time series data of the vertical displacement of the toe of one foot in a period of 65% to 70% of one walking cycle to the prediction model, the sarcopenia determination unit 113 acquires, from the prediction model, a determination result indicating whether or not the subject is a sarcopenia subject or a pre-sarcopenia subject.

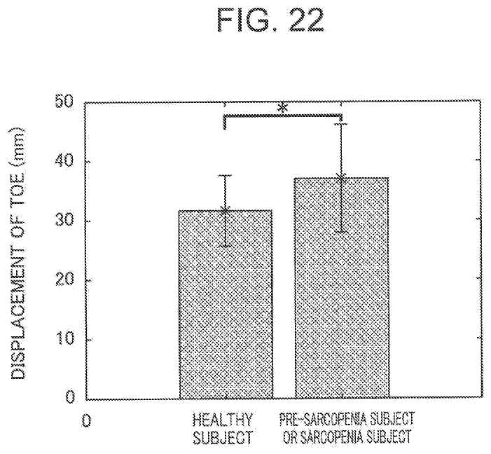

FIG. 22 is a view showing an average of mean values of time series data of the vertical displacement of the toe of one foot of sarcopenia subjects or pre-sarcopenia subjects and an average of mean values of time series data of the vertical displacement of the toe of one foot of healthy subjects in the fifth modification of the present embodiment.

As shown in FIG. 22, the average of mean values of time series data of the vertical displacement of the toe of one foot of the sarcopenia subjects or the pre-sarcopenia subjects in the period of 65% to 70% of one walking cycle was 37 mm, and the average of mean values of time series data of the vertical displacement of the toe of one foot of the healthy subjects in the period of 65% to 70% of one walking cycle was 31 mm.

Thus, in the period of 65% to 70% of one walking cycle, the average of mean values of time series data of the vertical displacement of the toe of one foot of the sarcopenia subjects or the pre-sarcopenia subjects is larger than the average of mean values of time series data of the vertical displacement of the toe of one foot of the healthy subjects. Therefore, a value between an average of the mean values of time series data of the vertical displacement of the toe of one foot in a period of 65% to 70% of one walking cycle of the sarcopenia subjects or the pre-sarcopenia subjects and an average of the mean values of time series data of the vertical displacement of the toe of one foot in a period of 65% to 70% of one walking cycle of the healthy subjects, having been experimentally obtained, may be stored in the memory 12 as a threshold value. The sarcopenia determination unit 113 may determine whether or not the subject is a sarcopenia subject or a pre-sarcopenia subject by comparing the mean value of time series data of the vertical displacement of the toe of one foot of the subject in a period of 65% to 70% of one walking cycle with the threshold value stored in advance.

Subsequently, the walking parameters in the sixth modification of the present embodiment will be described.

The walking parameter in the sixth modification of the present embodiment may be a mean value of time series data of the angle of the ankle joint of one foot in a predetermined period of the stance phase of one leg.

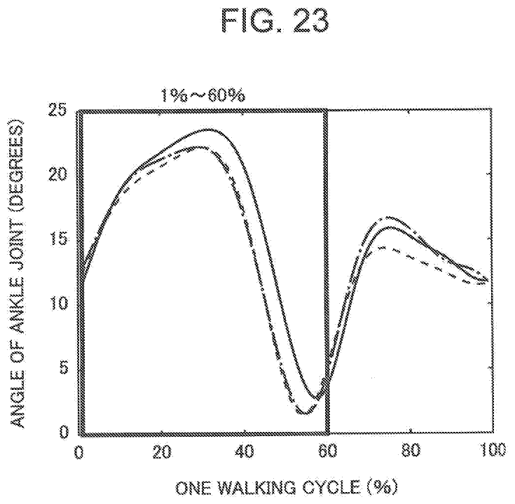

FIG. 23 is a view showing a change in the angle of the ankle joint of one foot in one walking cycle in a sixth modification of the present embodiment. In FIG. 23, the vertical axis represents the angle of the ankle joint, and the horizontal axis represents one normalized walking cycle. In addition, in FIG. 23, the dashed line represents an average waveform of the angles of one ankle joint of the healthy subjects, the dashed-dotted line represents an average waveform of the angles of one ankle joint of the pre-sarcopenia subjects, and the solid line represents an average waveform of the angles of one ankle joint of the sarcopenia subjects.

In the sixth modification of the present embodiment, similar to the above experiment, time series data of the angle of one ankle joint of each of the plurality of subjects was detected from the skeleton data of a plurality of subjects including a sarcopenia subject, a pre-sarcopenia subject, and a healthy subject. As shown in FIG. 2, an angle θ of the ankle joint is an angle formed in the sagittal plane by a straight line connecting the feature point 212 indicating the right ankle joint and the feature point 211 indicating the right knee joint and a straight line connecting the feature point 212 indicating the right ankle joint and the feature point 213 indicating the right toe.

In the experiment, one normalized walking cycle was divided into ten intervals, and the mean value of the angles of one ankle joint in one interval or two or more consecutive intervals was calculated for each subject. Then, a prediction model was created with which of a sarcopenia subject, a pre-sarcopenia subject, and a healthy subject the subject is as an objective variable, and with the mean value of the angles of one ankle joint in a period of 1% to 60% of one walking cycle as an explanatory variable. The prediction model was evaluated by cross validation. Leave-one-out cross validation was adopted as the cross validation. Then, an ROC curve of the prediction model in which a healthy subject and a sarcopenia subject were determined, and an ROC curve of the prediction model in which a healthy subject and a pre-sarcopenia subject were determined were calculated. Furthermore, the AUC value of each of the two ROC curves of the prediction model was calculated.

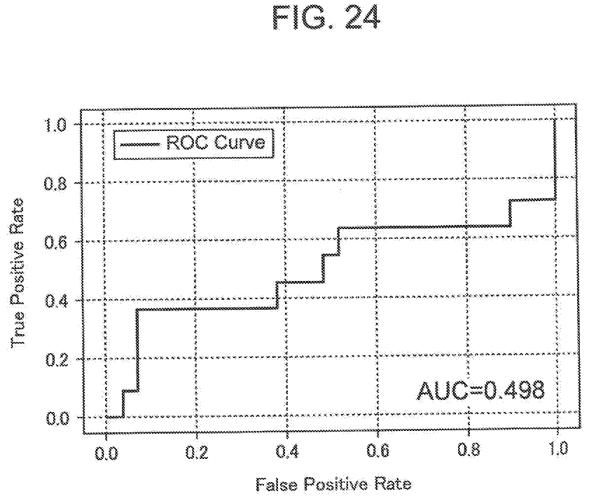

FIG. 24 is a view showing an ROC curve obtained as a result of determining a healthy subject and a sarcopenia subject using a prediction model in the sixth modification of the present embodiment.

The prediction model in the sixth modification of the present embodiment was created with which of a sarcopenia subject, a pre-sarcopenia subject, and a healthy subject the subject is as an objective variable, and with the mean value of the angles of one ankle joint in a period of 1% to 60% of one walking cycle as an explanatory variable. In FIG. 24, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 24 was obtained by plotting the true positive rate and the false positive rate of the prediction model created with the mean value of the angles of one ankle joint in a period of 1% to 60% of one walking cycle as an explanatory variable. The AUC value of the ROC curve shown in FIG. 24 was 0.498.

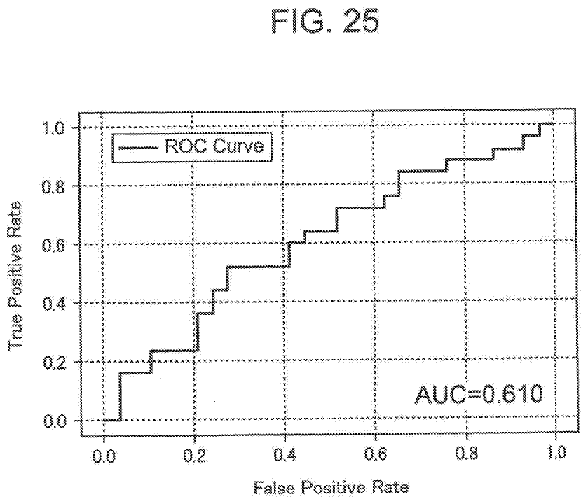

FIG. 25 is a view showing an ROC curve obtained as a result of determining a healthy subject and a pre-sarcopenia subject using the prediction model in the sixth modification of the present embodiment.

In FIG. 25, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 25 was obtained by plotting the true positive rate and the false positive rate of the prediction model created with the mean value of the angles of one ankle joint in a period of 1% to 60% of one walking cycle as an explanatory variable. The AUC value of the ROC curve shown in FIG. 25 was 0.610.

In the sixth modification of the present embodiment, the mean value of the angles of one ankle joint in a period of 1% to 60% of one walking cycle is determined as a walking parameter. In addition, the prediction model created with the mean value of the angles of one ankle joint in a period of 1% to 60% of one walking cycle as an explanatory variable is determined as a prediction model to be used by the sarcopenia determination unit 113.

The walking parameter detection unit 112 detects, from walking data, the angle of the ankle joint of one foot in the stance phase of one leg of the subject. The walking parameter detection unit 112 detects the angle of the ankle joint of one foot in the stance phase of the subject from the time series skeleton data corresponding to the one walking cycle having been clipped. In particular, the walking parameter detection unit 112 detects time series data of the angle of the ankle joint of one foot in a predetermined period of the stance phase of one leg. More specifically, the predetermined period is a period of 1% to 60% of one walking cycle. The walking parameter detection unit 112 detects time series data of the angle of the ankle joint of one foot in a period of 1% to 60% of one walking cycle. In addition, the walking parameter detection unit 112 calculates the mean value of time series data of the angle of the ankle joint of one foot in a period of 1% to 60% of one walking cycle.

It is to be noted that in the sixth modification of the present embodiment, since the one walking cycle is a period from when the right foot of the subject touches the ground to when the right foot touches the ground again, the walking parameter detection unit 112 detects the angle θ of the ankle joint of the right foot. In a case where one walking cycle is a period from when the left foot of the subject touches the ground to when the left foot touches the ground again, the walking parameter detection unit 112 may detect the angle θ of the ankle joint of the left foot.

The memory 12 stores in advance a prediction model generated with the angle of the ankle joint of one foot in a period of 1% to 60% of one walking cycle as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the time series data of the angle of the ankle joint of one foot in a period of 1% to 60% of one walking cycle as an explanatory variable. In particular, the memory 12 stores in advance a prediction model generated with the mean value of time series data of the angle of the ankle joint of one foot in a period of 1% to 60% of one walking cycle as an input value and with which of a sarcopenia subject, a pre-sarcopenia subject, and a healthy subject the subject is as an output value.

The sarcopenia determination unit 113 determines whether or not the subject has sarcopenia using the angle of the ankle joint of one foot in the stance phase. The sarcopenia determination unit 113 determines whether or not the subject has sarcopenia by inputting the angle of the ankle joint of one foot in the stance phase detected by the walking parameter detection unit 112 to a prediction model generated with the angle of the ankle joint of one foot in the stance phase as an input value and with whether or not the subject has sarcopenia as an output value.

In addition, the sarcopenia determination unit 113 determines whether or not the subject has sarcopenia using the mean value of time series data of the angle of the ankle joint of one foot in a predetermined period of the stance phase of one leg. The predetermined period is a period of 1% to 60% of one walking cycle. The sarcopenia determination unit 113 determines whether or not the subject has sarcopenia using the mean value of time series data of the angle of the ankle joint of one foot in a period of 1% to 60% of one walking cycle. By inputting the mean value of time series data of the angle of the ankle joint of one foot in a period of 1% to 60% of one walking cycle to the prediction model, the sarcopenia determination unit 113 acquires, from the prediction model, a determination result indicating whether or not the subject has sarcopenia.

In addition, the sarcopenia determination unit 113 determines which of a sarcopenia subject, a pre-sarcopenia subject, and a healthy subject the subject is by using the mean value of time series data of the angle of the ankle joint of one foot in a predetermined period of the stance phase of one leg. The predetermined period is a period of 1% to 60% of one walking cycle. More specifically, the sarcopenia determination unit 113 determines which of a sarcopenia subject, a pre-sarcopenia subject, and a healthy subject the subject is by using the mean value of time series data of the angle of the ankle joint of one foot in a period of 1% to 60% of one walking cycle. By inputting the mean value of time series data of the angle of the ankle joint of one foot in a period of 1% to 60% of one walking cycle to the prediction model, the sarcopenia determination unit 113 acquires, from the prediction model, a determination result indicating which of a sarcopenia subject, a pre-sarcopenia subject, or a healthy subject the subject is.

In addition, in the period of 1% to 60% of one walking cycle shown in FIG. 23, the average waveform of the angle of the ankle joint of the sarcopenia subjects is larger than the average waveform of the angle of the ankle joint of the pre-sarcopenia subjects. Therefore, a value between an average of the mean values of time series data of the angle of the ankle joint in a period of 1% to 60% of one walking cycle of the sarcopenia subjects and an average of the mean values of time series data of the angle of the ankle joint in a period of 1% to 60% of one walking cycle of the pre-sarcopenia subjects, having been experimentally obtained, may be stored in the memory 12 as the first threshold value. The sarcopenia determination unit 113 may determine whether or not the subject has sarcopenia by comparing the mean value of time series data of the angle of the ankle joint of the subject in a period of 1% to 60% of one walking cycle with the first threshold value stored in advance.

In addition, in the period of 1% to 60% of one walking cycle shown in FIG. 23, the average waveform of the angle of the ankle joint of the pre-sarcopenia subjects is larger than the average waveform of the angle of the ankle joint of the healthy subjects. Therefore, a value between an average of the mean values of time series data of the angle of the ankle joint in a period of 1% to 60% of one walking cycle of the pre-sarcopenia subjects and an average of the mean values of time series data of the angle of the ankle joint in a period of 1% to 60% of one walking cycle of the healthy subjects, having been experimentally obtained, may be stored in the memory 12 as the second threshold value. The sarcopenia determination unit 113 may determine whether or not the subject is a pre-sarcopenia subject by comparing the mean value of time series data of the angle of the ankle joint of the subject in a period of 1% to 60% of one walking cycle with the second threshold value stored in advance.

Subsequently, the walking parameter in the seventh modification of the present embodiment will be described.

The walking parameter in the seventh modification of the present embodiment may be a mean value of time series data of the angle of the ankle joint of one foot in a predetermined period of the swing phase of one leg.

In the seventh modification of the present embodiment, similar to the above experiment, time series data of the angle of one ankle joint of each of the plurality of subjects was detected from the skeleton data of a plurality of subjects including a sarcopenia subject, a pre-sarcopenia subject, and a healthy subject.

In the experiment, one normalized walking cycle was divided into ten intervals, and the mean value of the angles of one ankle joint in one interval or two or more consecutive intervals was calculated for each subject. Then, a prediction model was created with which of a sarcopenia subject, a pre-sarcopenia subject, and a healthy subject the subject is as an objective variable, and with the mean value of the angles of one ankle joint in a period of 61% to 100% of one walking cycle as an explanatory variable. The prediction model was evaluated by cross validation. Leave-one-out cross validation was adopted as the cross validation. Then, an ROC curve of the prediction model in which a healthy subject and a sarcopenia subject were determined, and an ROC curve of the prediction model in which a healthy subject and a pre-sarcopenia subject were determined were calculated. Furthermore, the AUC value of each of the two ROC curves of the prediction model was calculated.

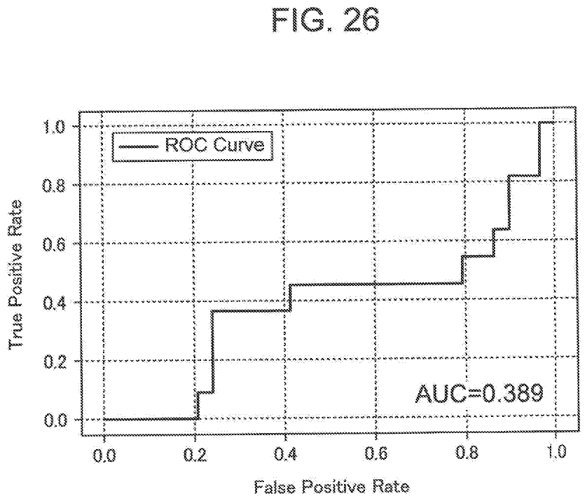

FIG. 26 is a view showing an ROC curve obtained as a result of determining a healthy subject and a sarcopenia subject using a prediction model in the seventh modification of the present embodiment.

The prediction model in the seventh modification of the present embodiment was created with which of a sarcopenia subject, a pre-sarcopenia subject, and a healthy subject the subject is as an objective variable, and with the mean value of the angles of one ankle joint in a period of 61% to 100% of one walking cycle as an explanatory variable. In FIG. 26, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 26 was obtained by plotting the true positive rate and the false positive rate of the prediction model created with the mean value of the angles of one ankle joint in a period of 61% to 100% of one walking cycle as an explanatory variable. The AUC value of the ROC curve shown in FIG. 26 was 0.389.

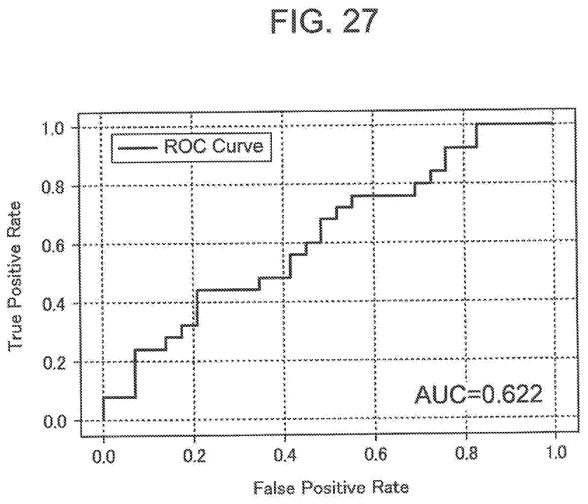

FIG. 27 is a view showing an ROC curve obtained as a result of determining a healthy subject and a pre-sarcopenia subject using the prediction model in the seventh modification of the present embodiment.

In FIG. 27, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 27 was obtained by plotting the true positive rate and the false positive rate of the prediction model created with the mean value of the angles of one ankle joint in a period of 61% to 100% of one walking cycle as an explanatory variable. The AUC value of the ROC curve shown in FIG. 27 was 0.622.

In the seventh modification of the present embodiment, the mean value of the angles of one ankle joint in a period of 61% to 100% of one walking cycle is determined as a walking parameter. In addition, the prediction model created with the mean value of the angles of one ankle joint in a period of 61% to 100% of one walking cycle as an explanatory variable is determined as a prediction model to be used by the sarcopenia determination unit 113.

The walking parameter detection unit 112 detects, from walking data, the angle of the ankle joint of one foot in the swing phase of one leg of the subject. The walking parameter detection unit 112 detects the angle of the ankle joint of one foot in the swing phase of the subject from the time series skeleton data corresponding to the one walking cycle having been clipped. In particular, the walking parameter detection unit 112 detects time series data of the angle of the ankle joint of one foot in a predetermined period of the swing phase of one leg. More specifically, the predetermined period is a period of 61% to 100% of one walking cycle. The walking parameter detection unit 112 detects time series data of the angle of the ankle joint of one foot in a period of 61% to 100% of one walking cycle. In addition, the walking parameter detection unit 112 calculates the mean value of time series data of the angle of the ankle joint of one foot in a period of 61% to 100% of one walking cycle.

The memory 12 stores in advance a prediction model generated with the angle of the ankle joint of one foot in a period of 61% to 100% of one walking cycle as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the time series data of the angle of the ankle joint of one foot in a period of 61% to 100% of one walking cycle as an explanatory variable. In particular, the memory 12 stores in advance a prediction model generated with the mean value of time series data of the angle of the ankle joint of one foot in a period of 61% to 100% of one walking cycle as an input value and with which of a sarcopenia subject, a pre-sarcopenia subject, and a healthy subject the subject is as an output value.

The sarcopenia determination unit 113 determines whether or not the subject has sarcopenia using the angle of the ankle joint of one foot in the swing phase. The sarcopenia determination unit 113 determines whether or not the subject has sarcopenia by inputting the angle of the ankle joint of one foot in the swing phase detected by the walking parameter detection unit 112 to a prediction model generated with the angle of the ankle joint of one foot in the swing phase as an input value and with whether or not the subject has sarcopenia as an output value.

In addition, the sarcopenia determination unit 113 determines whether or not the subject has sarcopenia using the mean value of time series data of the angle of the ankle joint of one foot in a predetermined period of the swing phase of one leg. The predetermined period is a period of 61% to 100% of one walking cycle. The sarcopenia determination unit 113 determines whether or not the subject has sarcopenia using the mean value of time series data of the angle of the ankle joint of one foot in a period of 61% to 100% of one walking cycle. By inputting the mean value of time series data of the angle of the ankle joint of one foot in a period of 61% to 100% of one walking cycle to the prediction model, the sarcopenia determination unit 113 acquires, from the prediction model, a determination result indicating whether or not the subject has sarcopenia.

In addition, the sarcopenia determination unit 113 determines which of a sarcopenia subject, a pre-sarcopenia subject, and a healthy subject the subject is by using the mean value of time series data of the angle of the ankle joint of one foot in a predetermined period of the swing phase of one leg. The predetermined period is a period of 61% to 100% of one walking cycle. More specifically, the sarcopenia determination unit 113 determines which of a sarcopenia subject, a pre-sarcopenia subject, and a healthy subject the subject is by using the mean value of time series data of the angle of the ankle joint of one foot in a period of 61% to 100% of one walking cycle. By inputting the mean value of time series data of the angle of the ankle joint of one foot in a period of 61% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Subsequently, the walking parameters in the eighth modification of the present embodiment will be described.

The walking parameter in the eighth modification of the present embodiment may be a mean value of time series data of the first angle of the ankle joint in the first period of the stance phase of one leg and a mean value of time series data of the second angle of the ankle joint in the second period of the swing phase of one leg.

In the eighth modification of the present embodiment, similar to the above experiment, time series data of the angle of one ankle joint of each of the plurality of subjects was detected from the skeleton data of a plurality of subjects including a sarcopenia subject, a pre-sarcopenia subject, and a healthy subject. In addition, a prediction model was created with which of a sarcopenia subject, a pre-sarcopenia subject, and a healthy subject the subject is as an objective variable, and with the mean value of the angles of one ankle joint in a period of 11% to 40% of one walking cycle and the mean value of the angles of one ankle joint in a period of 71% to 80% of one walking cycle as explanatory variables. The prediction model was evaluated by cross validation. Leave-one-out cross validation was adopted as the cross validation. Then, the ROC curve of the prediction model in which a healthy subject and a sarcopenia subject were determined was calculated. Furthermore, the AUC value of the ROC curve of the prediction model was calculated.

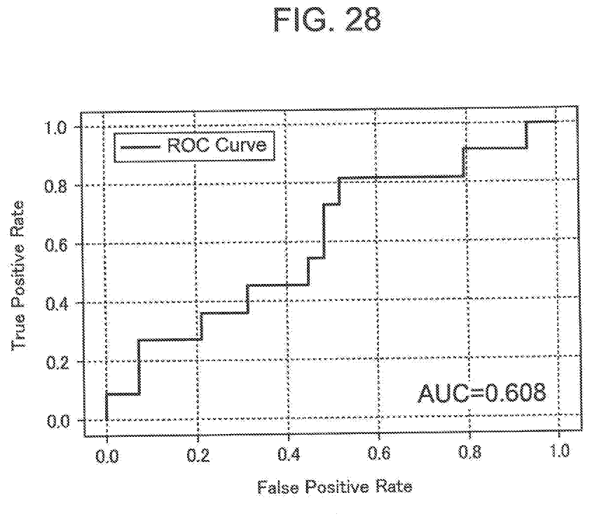

FIG. 28 is a view showing an ROC curve obtained as a result of determining a healthy subject and a sarcopenia subject using a prediction model in the eighth modification of the present embodiment.

The prediction model in the eighth modification of the present embodiment was created with which of a sarcopenia subject, a pre-sarcopenia subject, and a healthy subject the subject is as an objective variable, and with the mean value of the angles of one ankle joint in a period of 11% to 40% of one walking cycle and the mean value of the angles of one ankle joint in a period of 71% to 80% of one walking cycle as explanatory variables. In FIG. 28, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 28 was obtained by plotting the true positive rate and the false positive rate of the prediction model created with the mean value of the angles of one ankle joint in a period of 11% to 40% of one walking cycle and the mean value of the angles of one ankle joint in a period of 71% to 80% of one walking cycle as explanatory variables. The AUC value of the ROC curve shown in FIG. 28 was 0.608.

In the eighth modification of the present embodiment, the mean value of the angles of one ankle joint in a period of 11% to 40% of one walking cycle and the mean value of the angles of one ankle joint in a period of 71% to 80% of one walking cycle are determined as walking parameters. In addition, the prediction model created with the mean value of the angles of one ankle joint in a period of 11% to 40% of one walking cycle and the mean value of the angles of one ankle joint in a period of 71% to 80% of one walking cycle as explanatory variables is determined as a prediction model to be used by the sarcopenia determination unit 113.

The walking parameter detection unit 112 detects time series data of the first angle of the ankle joint in the first period of the stance phase of one leg and time series data of the second angle of the ankle joint in the second period of the swing phase of one leg. The first period is a period of 11% to 40% of one walking cycle, and the second period is a period of 71% to 80% of one walking cycle. The walking parameter detection unit 112 detects time series data of the angle of one ankle joint in a period of 11% to 40% of one walking cycle and time series data of the angle of one ankle joint in a period of 71% to 80% of one walking cycle. In addition, the walking parameter detection unit 112 calculates the mean value of time series data of the angle of one ankle joint in a period of 11% to 40% of one walking cycle and the mean value of time series data of the angle of one ankle joint in a period of 71% to 80% of one walking cycle.

The sarcopenia determination unit 113 determines whether or not the subject has sarcopenia using the mean value of time series data of the first angle of the ankle joint and the mean value of time series data of the second angle of the ankle joint.

The memory 12 stores in advance a prediction model generated with the mean value of time series data of the first angle of the ankle joint in the first period of the stance phase of one leg and the mean value of time series data of the second angle of the ankle joint in the second period of the swing phase of the one leg as input values and with which of a sarcopenia subject, a pre-sarcopenia subject, and a healthy subject the subject is as an output value. The memory 12 stores in advance a prediction model generated with the mean value of the angles of one ankle joint in a period of 11% to 40% of one walking cycle and the mean value of the angles of the one ankle joint in a period of 71% to 80% of one walking cycle as input values and with which of a sarcopenia subject, a pre-sarcopenia subject, and a healthy subject the subject is as an output value.

The sarcopenia determination unit 113 determines which of a sarcopenia subject, a pre-sarcopenia subject, and a healthy subject the subject is by using the mean value of time series data of the angle of one ankle joint in a period of 11% to 40% of one walking cycle and the mean value of time series data of the angle of one ankle joint in a period of 71% to 80% of one walking cycle. By inputting the mean value of time series data of the angle of one ankle joint in a period of 11% to 40% of one walking cycle and the mean value of time series data of the angle of one ankle joint in a period of 71% to 80% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining sarcopenia by the prediction model created using the mean value of the angles of the ankle joint in the stance phase in isolation was 0.498, and the AUC value obtained as a result of determining sarcopenia by the prediction model created using the mean value of the angles of the ankle joint in the swing phase in isolation was 0.389. On the other hand, the AUC value obtained as a result of determining sarcopenia by the prediction model created using the mean value of the angles of the ankle joint in the two periods was 0.608. Accordingly, it is possible to determine sarcopenia more accurately in a prediction model created using a mean value of the angles of the ankle joint in two periods than in a prediction model created using the mean value of the angles of the ankle joint in one period in isolation.

Subsequently, the walking parameters in the ninth modification of the present embodiment will be described.

The walking parameter in the ninth modification of the present embodiment may be the stride distance of one leg.

In the ninth modification of the present embodiment, similar to the above experiment, the stride distance of one leg of each of the plurality of subjects was detected from the skeleton data of a plurality of subjects including a sarcopenia subject, a pre-sarcopenia subject, and a healthy subject. The stride distance is the distance from a point where the heel of one foot touches the ground to a point where the heel of the one foot touches the ground again. In a case where one walking cycle is a period from when the right foot of the subject touches the ground to when the right foot touches the ground again, the stride distance is the distance from a point where the heel of the right foot touches the ground to a point where the heel of the right foot touches the ground again.

In the experiment, the stride distance of one leg was calculated for each subject. Then, a prediction model was created with which of a sarcopenia subject, a pre-sarcopenia subject, and a healthy subject the subject is as an objective variable, and with the stride distance of one leg in one walking cycle as an explanatory variable. The prediction model was evaluated by cross validation. Leave-one-out cross validation was adopted as the cross validation. Then, the ROC curve of the prediction model in which a healthy subject and a sarcopenia subject were determined was calculated. Furthermore, the AUC value of the ROC curve of the prediction model was calculated.

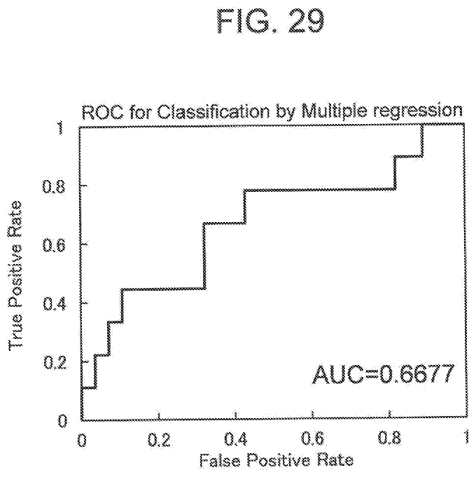

FIG. 29 is a view showing an ROC curve obtained as a result of determining a healthy subject and a sarcopenia subject using a prediction model in the ninth modification of the present embodiment.

The prediction model in the ninth modification of the present embodiment was created with which of a sarcopenia subject, a pre-sarcopenia subject, and a healthy subject the subject is as an objective variable, and with the stride distance of one leg in one walking cycle as an explanatory variable. In FIG. 29, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 29 was obtained by plotting the true positive rate and the false positive rate of the prediction model created with the stride distance of one leg in one walking cycle as an explanatory variable. The AUC value of the ROC curve shown in FIG. 29 was 0.6677.

In the ninth modification of the present embodiment, the stride distance of one leg in one walking cycle is determined as the walking parameter. In addition, the prediction model created with the stride distance of one leg in one walking cycle as an explanatory variable is determined as a prediction model to be used by the sarcopenia determination unit 113.

The walking parameter detection unit 112 detects, from walking data, the stride distance of one leg of the subject. The walking parameter detection unit 112 detects the stride distance of one leg of the subject from the time series skeleton data corresponding to the one walking cycle having been clipped.

It is to be noted that in the ninth modification of the present embodiment, since the one walking cycle is a period from when the right foot of the subject touches the ground to when the right foot of the subject touches the ground again, the walking parameter detection unit 112 detects the stride distance from the point where the heel of the right foot touches the ground to the point where the heel of the right foot touches the ground again. In a case where one walking cycle is a period from when the left foot of the subject touches the ground to when the left foot touches the ground again, the walking parameter detection unit 112 may detect the stride distance from the point where the heel of the left foot touches the ground to the point where the heel of the left foot touches the ground again. In addition, the walking parameter detection unit 112 may detect a plurality of stride distances in a plurality of walking cycles and calculate the mean value of the plurality of stride distances having been detected.

The memory 12 stores in advance a prediction model generated with the stride distance of one leg in one walking cycle as an input value and with which of a sarcopenia subject, a pre-sarcopenia subject, and a healthy subject the subject is as an output value. The prediction model is a regression model with which of a sarcopenia subject, a pre-sarcopenia subject, and a healthy subject the subject is as an objective variable, and with the stride distance of one leg in one walking cycle as an explanatory variable.

The sarcopenia determination unit 113 determines whether or not the subject has sarcopenia using the stride distance of one leg in one walking cycle. The sarcopenia determination unit 113 determines whether or not the subject has sarcopenia by inputting the stride distance of one leg in one walking cycle detected by the walking parameter detection unit 112 to a prediction model generated with the stride distance of one leg in one walking cycle as an input value and with whether or not the subject has sarcopenia as an output value. In addition, by inputting the stride distance of one leg in one walking cycle to the prediction model, the sarcopenia determination unit 113 acquires, from the prediction model, a determination result indicating whether or not the subject has sarcopenia.

In addition, the sarcopenia determination unit 113 determines which of a sarcopenia subject, a pre-sarcopenia subject, and a healthy subject the subject is by using the stride distance of one leg in one walking cycle. By inputting the stride distance of one leg in one walking cycle to the prediction model, the sarcopenia determination unit 113 acquires, from the prediction model, a determination result indicating which of a sarcopenia subject, a pre-sarcopenia subject, or a healthy subject the subject is.

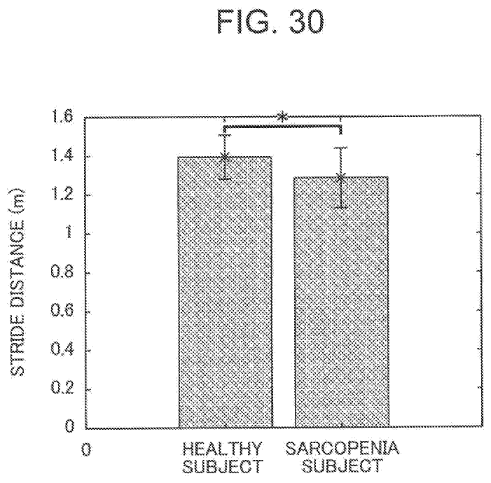

FIG. 30 is a view showing an average of the stride distances of one leg of the sarcopenia subjects and an average of the stride distances of one leg of the healthy subjects in the ninth modification of the present embodiment.

As shown in FIG. 30, the average of the stride distances of one leg of the sarcopenia subjects in one walking cycle was 1.28 m, and the average of the stride distances of one leg of the healthy subjects in one walking cycle was 1.39 m.

Thus, in one walking cycle, the average of the stride distances of one leg of the sarcopenia subjects is smaller than the average of the stride distances of one leg of the healthy subjects. Therefore, a value between an average of the stride distance of one leg in one walking cycle of the sarcopenia subjects and an average of the stride distance of one leg in one walking cycle of the healthy subjects, having been experimentally obtained, may be stored in the memory 12 as a threshold value. The sarcopenia determination unit 113 may determine whether or not the subject has sarcopenia by comparing the stride distance of one leg of the subject in one walking cycle with the threshold value stored in advance. When the stride distance of one leg is smaller than the threshold value, the sarcopenia determination unit 113 may determine that the subject has sarcopenia.

Subsequently, the walking parameters in the tenth modification of the present embodiment will be described.

The walking parameter in the tenth modification of the present embodiment may be a mean value of time series data of the vertical displacement of the toe of one foot in the first period of the stance phase of one leg, a mean value of time series data of the angle of the knee joint of one leg in the second period of the stance phase of one leg, a mean value of time series data of the angle of the knee joint of one leg in the third period of the swing phase of one leg, and a mean value of time series data of the angle of the knee joint of one leg in the fourth period of the swing phase of one leg.

In the tenth modification of the present embodiment, similar to the above experiment, time series data of the vertical displacement of the toe of one foot of each of the plurality of subjects and time series data of the angle of the knee joint of one leg of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the vertical displacements of the toe of one foot and the mean value of the angles of the knee joint of one leg in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the vertical displacement of the toe of one foot in the first period of the stance phase, the mean value of time series data of the angle of the knee joint of one leg in the second period of the stance phase, the mean value of time series data of the angle of the knee joint of one leg in the third period of the swing phase, and the mean value of time series data of the angle of the knee joint of one leg in the fourth period of the swing phase as explanatory variables. The first period is a period of 1% to 30% of one walking cycle, the second period is a period of 1% to 50% of one walking cycle, the third period is a period of 61% to 70% of one walking cycle, and the fourth period is a period of 81% to 100% of one walking cycle. The prediction model was evaluated by cross validation. Leave-one-out cross validation was adopted as the cross validation. Then, the ROC curve of the prediction model in which a healthy subject and a sarcopenia subject were determined was calculated. Furthermore, the AUC value of the ROC curve of the prediction model was calculated.

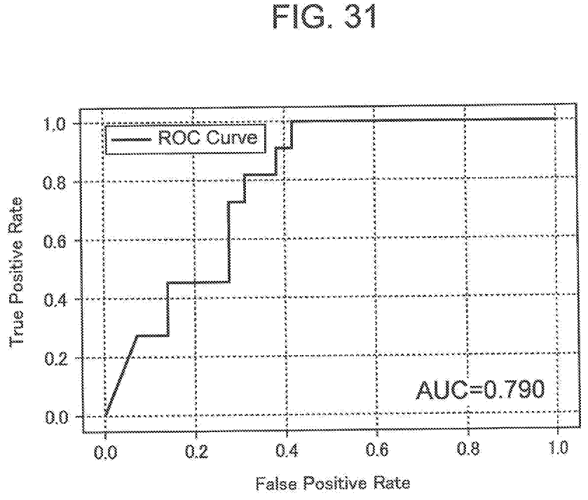

FIG. 31 is a view showing an ROC curve obtained as a result of determining a healthy subject and a sarcopenia subject using a prediction model in the tenth modification of the present embodiment.

The prediction model in the tenth modification of the present embodiment was created with which of a sarcopenia subject, a pre-sarcopenia subject, and a healthy subject the subject is as an objective variable, and with the mean value of the vertical displacements of the toe of one foot in a period of 1% to 30% of one walking cycle and each of the mean values of the angles of one knee joint in each of periods of 1% to 50%, 61% to 70%, and 81% to 100% of one walking cycle as explanatory variables. In FIG. 31, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 31 was obtained by plotting the true positive rate and the false positive rate of the prediction model created with the mean value of the vertical displacements of the toe of one foot in a period of 1% to 30% of one walking cycle, the mean value of the angles of one knee joint in a period of 1% to 50% of one walking cycle, the mean value of the angles of one knee joint in a period of 61% to 70% of one walking cycle, and the mean value of the angles of one knee joint in a period of 81% to 100% of one walking cycle as explanatory variables. The AUC value of the ROC curve shown in FIG. 31 was 0.790.

In the tenth modification of the present embodiment, the mean value of the vertical displacements of the toe of one foot in a period of 1% to 30% of one walking cycle, the mean value of the angles of one knee joint in a period of 1% to 50% of one walking cycle, the mean value of the angles of one knee joint in a period of 61% to 70% of one walking cycle, and the mean value of the angles of one knee joint in a period of 81% to 100% of one walking cycle are determined as walking parameters. In addition, the prediction model created with the mean value of the vertical displacements of the toe of one foot in a period of 1% to 30% of one walking cycle, the mean value of the angles of one knee joint in a period of 1% to 50% of one walking cycle, the mean value of the angles of one knee joint in a period of 61% to 70% of one walking cycle, and the mean value of the angles of one knee joint in a period of 81% to 100% of one walking cycle as explanatory variables is determined as a prediction model to be used by the sarcopenia determination unit 113.

The walking parameter detection unit 112 detects time series data of the vertical displacement of the toe of one foot in the first period of the stance phase, time series data of the angle of the knee joint of one leg in the second period of the stance phase, time series data of the angle of the knee joint of one leg in the third period of the swing phase, and time series data of the angle of the knee joint of one leg in the fourth period of the swing phase. The first period is a period of 1% to 30% of one walking cycle, the second period is a period of 1% to 50% of one walking cycle, the third period is a period of 61% to 70% of one walking cycle, and the fourth period is a period of 81% to 100% of one walking cycle. The walking parameter detection unit 112 detects time series data of the vertical displacement of the toe of one foot in a period of 1% to 30% of one walking cycle, time series data of the angle of one knee joint in a period of 1% to 50% of one walking cycle, time series data of the angle of one knee joint in a period of 61% to 70% of one walking cycle, and time series data of the angle of one knee joint in a period of 81% to 100% of one walking cycle. In addition, the walking parameter detection unit 112 calculates the mean value of time series data of the vertical displacement of the toe of one foot in a period of 1% to 30% of one walking cycle, the mean value of time series data of the angle of one knee joint in a period of 1% to 50% of one walking cycle, the mean value of time series data of the angle of one knee joint in a period of 61% to 70% of one walking cycle, and the mean value of time series data of the angle of one knee joint in a period of 81% to 100% of one walking cycle.

The memory 12 stores in advance a prediction model generated with the mean value of time series data of the vertical displacement of the toe of one foot in the first period of the stance phase, the mean value of time series data of the angle of the knee joint of the one leg in the second period of the stance phase, the mean value of time series data of the angle of the knee joint of one leg in the third period of the swing phase, and the mean value of time series data of the angle of the knee joint of the one leg in the fourth period of the swing phase as input values and with which of a sarcopenia subject, a pre-sarcopenia subject, and a healthy subject the subject is as an output value. The memory 12 stores in advance a prediction model generated with the mean value of time series data of the vertical displacement of the toe of one foot in a period of 1% to 30% of one walking cycle, the mean value of time series data of the angle of one knee joint in a period of 1% to 50% of one walking cycle, the mean value of time series data of the angle of one knee joint in a period of 61% to 70% of one walking cycle, and the mean value of time series data of the angle of one knee joint in a period of 81% to 100% of one walking cycle as input values and with which of a sarcopenia subject, a pre-sarcopenia subject, and a healthy subject the subject is as an output value.

The sarcopenia determination unit 113 determines whether or not the subject has sarcopenia using the mean value of time series data of the vertical displacement of the toe of one foot in the first period and each of the mean values of time series data of the angle of the knee joint of one leg in each of the second period, the third period, and the fourth period.

The sarcopenia determination unit 113 determines which of a sarcopenia subject, a pre-sarcopenia subject, and a healthy subject the subject is by using the mean value of time series data of the vertical displacement of the toe of one foot in a period of 1% to 30% of one walking cycle, the mean value of time series data of the angle of one knee joint in a period of 1% to 50% of one walking cycle, the mean value of time series data of the angle of one knee joint in a period of 61% to 70% of one walking cycle, and the mean value of time series data of the angle of one knee joint in a period of 81% to 100% of one walking cycle. By inputting the mean value of time series data of the vertical displacement of the toe of one foot in a period of 1% to 30% of one walking cycle, the mean value of time series data of the angle of one knee joint in a period of 1% to 50% of one walking cycle, the mean value of time series data of the angle of one knee joint in a period of 61% to 70% of one walking cycle, and the mean value of time series data of the angle of one knee joint in a period of 81% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining sarcopenia by the prediction model created using the vertical displacement of the toe of one foot in the stance phase in isolation was 0.636, the AUC value obtained as a result of determining sarcopenia by the prediction model created using the angle of the knee joint of one leg in the stance phase in isolation was 0.586, and the AUC value obtained as a result of determining sarcopenia by the prediction model created using the angle of the knee joint of one leg in the swing phase in isolation was 0.699. On the other hand, the AUC value obtained as a result of determining sarcopenia by the prediction model created using the vertical displacement of the toe of one foot in the stance phase, the angle of the knee joint of one leg in the stance phase, and the angle of the knee joint of one leg in the swing phase was 0.790.

Accordingly, it is possible to determine sarcopenia more accurately in a prediction model created using a vertical displacement of the toe of one foot in the stance phase, an angle of the knee joint of one leg in the stance phase, and an angle of the knee joint of one leg in the swing phase than in a prediction model created using each of the vertical displacement of the toe of one foot in the stance phase, the angle of the knee joint of one leg in the stance phase, and the angle of the knee joint of one leg in the swing phase in isolation.

Subsequently, the walking parameters in the eleventh modification of the present embodiment will be described.

The walking parameter in the eleventh modification of the present embodiment may be a mean value of time series data of the vertical displacement of the toe of one foot in the first period of the stance phase of one leg, a mean value of time series data of the angle of the knee joint of one leg in the second period of the stance phase of one leg, a mean value of time series data of the angle of the knee joint of one leg in the third period of the stance phase of one leg, and a mean value of time series data of the angle of the knee joint of one leg in the fourth period of the swing phase of one leg.

In the eleventh modification of the present embodiment, similar to the above experiment, time series data of the vertical displacement of the toe of one foot of each of the plurality of subjects and time series data of the angle of the knee joint of one leg of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the vertical displacements of the toe of one foot and the mean value of the angles of the knee joint of one leg in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the vertical displacement of the toe of one foot in the first period of the stance phase, the mean value of time series data of the angle of the knee joint of one leg in the second period of the stance phase, the mean value of time series data of the angle of the knee joint of one leg in the third period of the stance phase, and the mean value of time series data of the angle of the knee joint of one leg in the fourth period of the swing phase as explanatory variables. The first period is a period of 31% to 40% of one walking cycle, the second period is a period of 1% to 10% of one walking cycle, the third period is a period of 41% to 50% of one walking cycle, and the fourth period is a period of 71% to 100% of one walking cycle. The prediction model was evaluated by cross validation. Leave-one-out cross validation was adopted as the cross validation. Then, the ROC curve of the prediction model in which a healthy subject and a pre-sarcopenia subject were determined was calculated. Furthermore, the AUC value of the ROC curve of the prediction model was calculated.

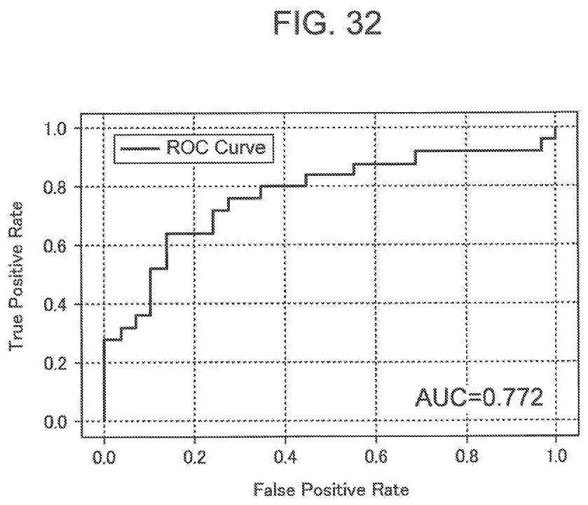

FIG. 32 is a view showing an ROC curve obtained as a result of determining a healthy subject and a pre-sarcopenia subject using the prediction model in the eleventh modification of the present embodiment.

The prediction model in the eleventh modification of the present embodiment was created with which of a sarcopenia subject, a pre-sarcopenia subject, and a healthy subject the subject is as an objective variable, and with the mean value of the vertical displacements of the toe of one foot in a period of 31% to 40% of one walking cycle and each of the mean values of the angles of one knee joint in each of periods of 1% to 10%, 41% to 50%, and 71% to 100% of one walking cycle as explanatory variables. In FIG. 32, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 32 was obtained by plotting the true positive rate and the false positive rate of the prediction model created with the mean value of the vertical displacements of the toe of one foot in a period of 31% to 40% of one walking cycle, the mean value of the angles of one knee joint in a period of 1% to 10% of one walking cycle, the mean value of the angles of one knee joint in a period of 41% to 50% of one walking cycle, and the mean value of the angles of one knee joint in a period of 71% to 100% of one walking cycle as explanatory variables. The AUC value of the ROC curve shown in FIG. 32 was 0.772.

In the eleventh modification of the present embodiment, the mean value of the vertical displacements of the toe of one foot in a period of 31% to 40% of one walking cycle, the mean value of the angles of one knee joint in a period of 1% to 10% of one walking cycle, the mean value of the angles of one knee joint in a period of 41% to 50% of one walking cycle, and the mean value of the angles of one knee joint in a period of 71% to 100% of one walking cycle are determined as walking parameters. In addition, the prediction model created with the mean value of the vertical displacements of the toe of one foot in a period of 31% to 40% of one walking cycle, the mean value of the angles of one knee joint in a period of 1% to 10% of one walking cycle, the mean value of the angles of one knee joint in a period of 41% to 50% of one walking cycle, and the mean value of the angles of one knee joint in a period of 71% to 100% of one walking cycle as explanatory variables is determined as a prediction model to be used by the sarcopenia determination unit 113.

The walking parameter detection unit 112 detects time series data of the vertical displacement of the toe of one foot in the first period of the stance phase, time series data of the angle of the knee joint of one leg in the second period of the stance phase, time series data of the angle of the knee joint of one leg in the third period of the stance phase, and time series data of the angle of the knee joint of one leg in the fourth period of the swing phase. The first period is a period of 31% to 40% of one walking cycle, the second period is a period of 1% to 10% of one walking cycle, the third period is a period of 41% to 50% of one walking cycle, and the fourth period is a period of 71% to 100% of one walking cycle.

The walking parameter detection unit 112 detects time series data of the vertical displacement of the toe of one foot in a period of 31% to 40% of one walking cycle, time series data of the angle of one knee joint in a period of 1% to 10% of one walking cycle, time series data of the angle of one knee joint in a period of 41% to 50% of one walking cycle, and time series data of the angle of one knee joint in a period of 71% to 100% of one walking cycle.

In addition, the walking parameter detection unit 112 calculates the mean value of time series data of the vertical displacement of the toe of one foot in a period of 31% to 40% of one walking cycle, the mean value of time series data of the angle of one knee joint in a period of 1% to 10% of one walking cycle, the mean value of time series data of the angle of one knee joint in a period of 41% to 50% of one walking cycle, and the mean value of time series data of the angle of one knee joint in a period of 71% to 100% of one walking cycle.

The memory 12 stores in advance a prediction model generated with the mean value of time series data of the vertical displacement of the toe of one foot in the first period of the stance phase, the mean value of time series data of the angle of the knee joint of one leg in the second period of the stance phase, the mean value of time series data of the angle of the knee joint of one leg in the third period of the stance phase, and the mean value of time series data of the angle of the knee joint of the one leg in the fourth period of the swing phase as input values and with which of a sarcopenia subject, a pre-sarcopenia subject, and a healthy subject the subject is as an output value.

The memory 12 stores in advance a prediction model generated with the mean value of time series data of the vertical displacement of the toe of one foot in a period of 31% to 40% of one walking cycle, the mean value of time series data of the angle of one knee joint in a period of 1% to 10% of one walking cycle, the mean value of time series data of the angle of one knee joint in a period of 41% to 50% of one walking cycle, and the mean value of time series data of the angle of one knee joint in a period of 71% to 100% of one walking cycle as input values and with which of a sarcopenia subject, a pre-sarcopenia subject, and a healthy subject the subject is as an output value.

The sarcopenia determination unit 113 determines whether or not the subject has sarcopenia using the mean value of time series data of the vertical displacement of the toe of one foot in the first period and each of the mean values of time series data of the angle of the knee joint of one leg in each of the second period, the third period, and the fourth period.

The sarcopenia determination unit 113 determines which of a sarcopenia subject, a pre-sarcopenia subject, and a healthy subject the subject is by using the mean value of time series data of the vertical displacement of the toe of one foot in a period of 31% to 40% of one walking cycle, the mean value of time series data of the angle of one knee joint in a period of 1% to 10% of one walking cycle, the mean value of time series data of the angle of one knee joint in a period of 41% to 50% of one walking cycle, and the mean value of time series data of the angle of one knee joint in a period of 71% to 100% of one walking cycle.

By inputting the mean value of time series data of the vertical displacement of the toe of one foot in a period of 31% to 40% of one walking cycle, the mean value of time series data of the angle of one knee joint in a period of 1% to 10% of one walking cycle, the mean value of time series data of the angle of one knee joint in a period of 41% to 50% of one walking cycle, and the mean value of time series data of the angle of one knee joint in a period of 71% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining a pre-sarcopenia subject by the prediction model created using the vertical displacement of the toe of one foot in the stance phase in isolation was 0.560, the AUC value obtained as a result of determining a pre-sarcopenia subject by the prediction model created using the angle of the knee joint of one leg in the stance phase in isolation was 0.537, and the AUC value obtained as a result of determining a pre-sarcopenia subject by the prediction model created using the angle of the knee joint of one leg in the swing phase in isolation was 0.604. On the other hand, the AUC value obtained as a result of determining a pre-sarcopenia subject by the prediction model created using the vertical displacement of the toe of one foot in the stance phase, the angle of the knee joint of one leg in the stance phase, and the angle of the knee joint of one leg in the swing phase was 0.772.

Accordingly, it is possible to determine a pre-sarcopenia subject more accurately in a prediction model created using a vertical displacement of the toe of one foot in the stance phase, an angle of the knee joint of one leg in the stance phase, and an angle of the knee joint of one leg in the swing phase than in a prediction model created using each of the vertical displacement of the toe of one foot in the stance phase, the angle of the knee joint of one leg in the stance phase, and the angle of the knee joint of one leg in the swing phase in isolation.

Subsequently, the walking parameters in the twelfth modification of the present embodiment will be described.

The walking parameter in the twelfth modification of the present embodiment may be a mean value of time series data of the vertical displacement of the toe of one foot in the first period of the swing phase of one leg and a mean value of time series data of the angle of the knee joint of one leg in the second period of the stance phase of one leg.

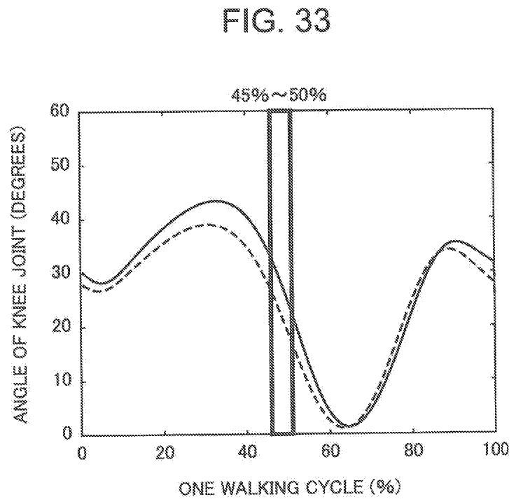

FIG. 33 is a view showing a change in the angle of the knee joint of one leg in one walking cycle in the twelfth modification of the present embodiment. In FIG. 33, the vertical axis represents the angle of the knee joint, and the horizontal axis represents one normalized walking cycle. In addition, in FIG. 33, the dashed line represents an average waveform of the angles of the knee joint of one leg of the healthy subjects, and the solid line represents an average waveform of the angles of the knee joint of one leg of the sarcopenia subjects or the pre-sarcopenia subjects.

It is to be noted that in the twelfth modification of the present embodiment, it is determined whether or not the subject is a sarcopenia subject or a pre-sarcopenia subject. It is to be noted that a subject who is neither a sarcopenia subject nor a pre-sarcopenia subject is determined to be a healthy subject.

In addition, the average waveform of the vertical displacements of the toe of one foot of the healthy subjects, and the average waveform of the vertical displacements of the toe of one foot of the sarcopenia subjects or the pre-sarcopenia subjects are shown in FIG. 20.

In the twelfth modification of the present embodiment, similar to the above experiment, time series data of the vertical displacement of the toe of one foot of each of the plurality of subjects and time series data of the angle of the knee joint of one leg of each of the plurality of subjects were detected from the skeleton data of a plurality of subjects including a sarcopenia subject, a pre-sarcopenia subject, and a healthy subject. In addition, a prediction model was created with whether or not the subject is a sarcopenia subject or a pre-sarcopenia subject as an objective variable, and with the mean value of time series data of the vertical displacement of the toe of one foot in the first period of the swing phase and the mean value of time series data of the angle of the knee joint of one leg in the second period of the stance phase as explanatory variables. The first period is a period of 65% to 70% of one walking cycle, and the second period is a period of 45% to 50% of one walking cycle. The prediction model was evaluated by cross validation. Leave-one-out cross validation was adopted as the cross validation. Then, the ROC curve of the prediction model in which whether or not to be a sarcopenia subject or a pre-sarcopenia subject was determined was calculated. Furthermore, the AUC value of the ROC curve of the prediction model was calculated.

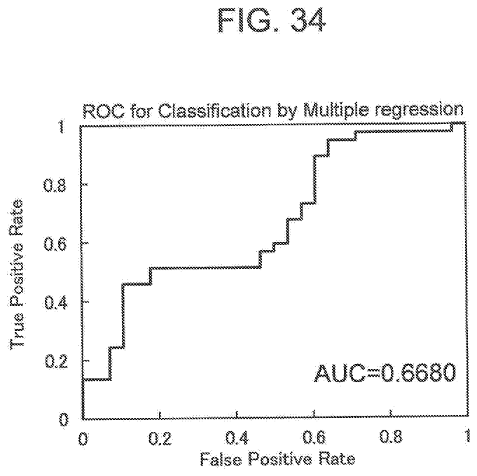

FIG. 34 is a view showing an ROC curve obtained as a result of determining whether or not to be a sarcopenia subject or a pre-sarcopenia subject using a prediction model in the twelfth modification of the present embodiment.

The prediction model in the twelfth modification of the present embodiment was created with whether or not the subject is a sarcopenia subject or a pre-sarcopenia subject as an objective variable, and with the mean value of the vertical displacements of the toe of one foot in a period of 65% to 70% of one walking cycle and the mean value of the angles of one knee joint in a period of 45% to 50% of one walking cycle as explanatory variables. In FIG. 34, the vertical axis represents the true positive rate, and the horizontal axis represents the false positive rate. The true positive rate indicates a ratio at which the prediction model has correctly determined a sarcopenia subject or a pre-sarcopenia subject as a sarcopenia subject or a pre-sarcopenia subject, and the false positive rate indicates a ratio at which the prediction model has incorrectly determined a healthy subject as a sarcopenia subject or a pre-sarcopenia subject.

The ROC curve shown in FIG. 34 was obtained by plotting the true positive rate and the false positive rate of the prediction model created with the mean value of the vertical displacements of the toe of one foot in a period of 65% to 70% of one walking cycle and the mean value of the angles of one knee joint in a period of 45% to 50% of one walking cycle as explanatory variables. The AUC value of the ROC curve shown in FIG. 34 was 0.6680.

In the twelfth modification of the present embodiment, the mean value of the vertical displacements of the toe of one foot in a period of 65% to 70% of one walking cycle and the mean value of the angles of one knee joint in a period of 45% to 50% of one walking cycle are determined as walking parameters. In addition, the prediction model created with the mean value of the vertical displacements of the toe of one foot in a period of 65% to 70% of one walking cycle and the mean value of the angles of one knee joint in a period of 45% to 50% of one walking cycle as explanatory variables is determined as a prediction model to be used by the sarcopenia determination unit 113.

The walking parameter detection unit 112 detects time series data of the vertical displacement of the toe of one foot in the first period of the swing phase and time series data of the angle of the knee joint of one leg in the second period of the stance phase. The first period is a period of 65% to 70% of one walking cycle, and the second period is a period of 45% to 50% of one walking cycle. The walking parameter detection unit 112 detects time series data of the vertical displacement of the toe of one foot in a period of 65% to 70% of one walking cycle and time series data of the angle of one knee joint in a period of 45% to 50% of one walking cycle. In addition, the walking parameter detection unit 112 calculates the mean value of time series data of the vertical displacement of the toe of one foot in a period of 65% to 70% of one walking cycle, the mean value of time series data of the angle of one knee joint in a period of 45% to 50% of one walking cycle.

The memory 12 stores in advance a prediction model generated with the mean value of time series data of the vertical displacement of the toe of one foot in the first period of the swing phase and the mean value of time series data of the angle of the knee joint of one leg in the second period of the stance phase as input values and with whether or not the subject is a sarcopenia subject or a pre-sarcopenia subject as an output value. The memory 12 stores in advance a prediction model generated with the mean value of time series data of the vertical displacement of the toe of one foot in a period of 65% to 70% of one walking cycle and the mean value of time series data of the angle of one knee joint in a period of 45% to 50% of one walking cycle as input values and with whether or not the subject is a sarcopenia subject or a pre-sarcopenia subject as an output value.

The sarcopenia determination unit 113 determines whether or not the subject has sarcopenia using the mean value of time series data of the vertical displacement of the toe of one foot in the first period and the mean value of time series data of the angle of the knee joint of one leg in the second period.

The sarcopenia determination unit 113 determines whether or not the subject is a sarcopenia subject or a pre-sarcopenia subject by using the mean value of time series data of the vertical displacement of the toe of one foot in a period of 65% to 70% of one walking cycle and the mean value of time series data of the angle of one knee joint in a period of 45% to 50% of one walking cycle. By inputting the mean value of time series data of the vertical displacement of the toe of one foot in a period of 65% to 70% of one walking cycle and the mean value of time series data of the angle of one knee joint in a period of 45% to 50% of one walking cycle to the prediction model, the sarcopenia determination unit 113 acquires, from the prediction model, a determination result indicating whether or not the subject is a sarcopenia subject or a pre-sarcopenia subject.

Subsequently, the walking parameters in the thirteenth modification of the present embodiment will be described.

The walking parameter in the thirteenth modification of the present embodiment may be a mean value of time series data of the vertical displacement of the toe of one foot in the first period of the stance phase of one leg, a mean value of time series data of the angle of the ankle joint of one foot in the second period of the stance phase of one leg, a mean value of time series data of the angle of the ankle joint of one foot in the third period of the stance phase of one leg, a mean value of time series data of the angle of the ankle joint of one foot in the fourth period of the swing phase of one leg, and a mean value of time series data of the angle of the ankle joint of one foot in the fifth period of the swing phase of one leg.

In the thirteenth modification of the present embodiment, similar to the above experiment, time series data of the vertical displacement of the toe of one foot of each of the plurality of subjects and time series data of the angle of the ankle joint of one foot of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the vertical displacements of the toe of one foot and the mean value of the angles of the ankle joint of one foot in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the vertical displacement of the toe of one foot in the first period of the stance phase, the mean value of time series data of the angle of the ankle joint of one foot in the second period of the stance phase, the mean value of time series data of the angle of the ankle joint of one foot in the third period of the stance phase, the mean value of time series data of the angle of the ankle joint of one foot in the fourth period of the swing phase, and the mean value of time series data of the angle of the ankle joint of one foot in the fifth period of the swing phase as explanatory variables. The first period is a period of 1% to 50% of one walking cycle, the second period is a period of 1% to 10% of one walking cycle, the third period is a period of 21% to 60% of one walking cycle, the fourth period is a period of 71% to 80% of one walking cycle, and the fifth period is a period of 91% to 100% of one walking cycle. The prediction model was evaluated by cross validation. Leave-one-out cross validation was adopted as the cross validation. Then, the ROC curve of the prediction model in which a healthy subject and a sarcopenia subject were determined was calculated. Furthermore, the AUC value of the ROC curve of the prediction model was calculated.

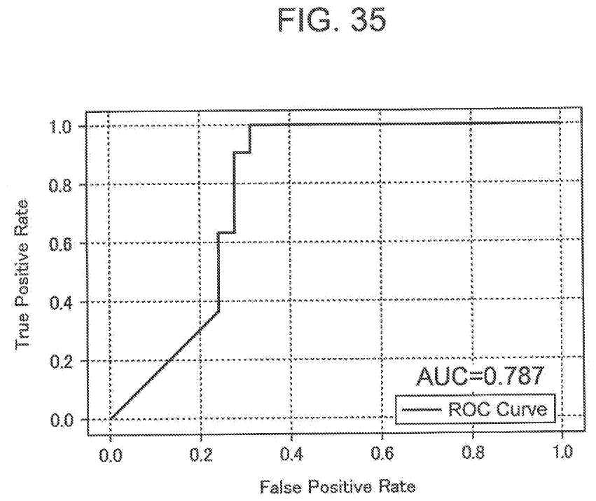

FIG. 35 is a view showing an ROC curve obtained as a result of determining a healthy subject and a sarcopenia subject using a prediction model in the thirteenth modification of the present embodiment.

The prediction model in the thirteenth modification of the present embodiment was created with which of a sarcopenia subject, a pre-sarcopenia subject, and a healthy subject the subject is as an objective variable, and with the mean value of the vertical displacements of the toe of one foot in a period of 1% to 50% of one walking cycle and each of the mean values of the angles of one ankle joint in each of periods of 1% to 10%, 21% to 60%, 71% to 80%, and 91% to 100% of one walking cycle as explanatory variables. In FIG. 35, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 35 was obtained by plotting the true positive rate and the false positive rate of the prediction model created with the mean value of the vertical displacements of the toe of one foot in a period of 1% to 50% of one walking cycle, the mean value of the angles of one ankle joint in a period of 1% to 10% of one walking cycle, the mean value of the angles of one ankle joint in a period of 21% to 60% of one walking cycle, the mean value of the angles of one ankle joint in a period of 71% to 80% of one walking cycle, and the mean value of the angles of one ankle joint in a period of 91% to 100% of one walking cycle as explanatory variables. The AUC value of the ROC curve shown in FIG. 35 was 0.787.

In the thirteenth modification of the present embodiment, the mean value of the vertical displacements of the toe of one foot in a period of 1% to 50% of one walking cycle, the mean value of the angles of one ankle joint in a period of 1% to 10% of one walking cycle, the mean value of the angles of one ankle joint in a period of 21% to 60% of one walking cycle, the mean value of the angles of one ankle joint in a period of 71% to 80% of one walking cycle, and the mean value of the angles of one ankle joint in a period of 91% to 100% of one walking cycle are determined as walking parameters. In addition, the prediction model created with the mean value of the vertical displacements of the toe of one foot in a period of 1% to 50% of one walking cycle, the mean value of the angles of one ankle joint in a period of 1% to 10% of one walking cycle, the mean value of the angles of one ankle joint in a period of 21% to 60% of one walking cycle, the mean value of the angles of one ankle joint in a period of 71% to 80% of one walking cycle, and the mean value of the angles of one ankle joint in a period of 91% to 100% of one walking cycle as explanatory variables is determined as a prediction model to be used by the sarcopenia determination unit 113.

The walking parameter detection unit 112 detects time series data of the vertical displacement of the toe of one foot in the first period of the stance phase, time series data of the angle of the ankle joint of one foot in the second period of the stance phase, time series data of the angle of the ankle joint of one foot in the third period of the stance phase, time series data of the angle of the ankle joint of one foot in the fourth period of the swing phase, and time series data of the angle of the ankle joint of one foot in the fifth period of the swing phase. The first period is a period of 1% to 50% of one walking cycle, the second period is a period of 1% to 10% of one walking cycle, the third period is a period of 21% to 60% of one walking cycle, the fourth period is a period of 71% to 80% of one walking cycle, and the fifth period is a period of 91% to 100% of one walking cycle.

The walking parameter detection unit 112 detects time series data of the vertical displacement of the toe of one foot in a period of 1% to 50% of one walking cycle, time series data of the angle of one ankle joint in a period of 1% to 10% of one walking cycle, time series data of the angle of one ankle joint in a period of 21% to 60% of one walking cycle, time series data of the angle of one ankle joint in a period of 71% to 80% of one walking cycle, and time series data of the angle of one ankle joint in a period of 91% to 100% of one walking cycle.

In addition, the walking parameter detection unit 112 calculates the mean value of time series data of the vertical displacement of the toe of one foot in a period of 1% to 50% of one walking cycle, the mean value of time series data of the angle of one ankle joint in a period of 1% to 10% of one walking cycle, the mean value of time series data of the angle of one ankle joint in a period of 21% to 60% of one walking cycle, the mean value of time series data of the angle of one ankle joint in a period of 71% to 80% of one walking cycle, and the mean value of time series data of the angle of one ankle joint in a period of 91% to 100% of one walking cycle.

The memory 12 stores in advance a prediction model generated with the mean value of time series data of the vertical displacement of the toe of one foot in the first period of the stance phase, the mean value of time series data of the angle of the ankle joint of the one foot in the second period of the stance phase, the mean value of time series data of the angle of the ankle joint of the one foot in the third period of the stance phase, the mean value of time series data of the angle of the ankle joint of the one foot in the fourth period of the swing phase, and the mean value of time series data of the angle of the ankle joint of the one foot in the fifth period of the swing phase as input values and with which of a sarcopenia subject, a pre-sarcopenia subject, and a healthy subject the subject is as an output value.

The memory 12 stores in advance a prediction model generated with the mean value of time series data of the vertical displacement of the toe of one foot in a period of 1% to 50% of one walking cycle, the mean value of time series data of the angle of one ankle joint in a period of 1% to 10% of one walking cycle, the mean value of time series data of the angle of one ankle joint in a period of 21% to 60% of one walking cycle, the mean value of time series data of the angle of one ankle joint in a period of 71% to 80% of one walking cycle, and the mean value of time series data of the angle of one ankle joint in a period of 91% to 100% of one walking cycle as input values and with which of a sarcopenia subject, a pre-sarcopenia subject, and a healthy subject the subject is as an output value.

The sarcopenia determination unit 113 determines whether or not the subject has sarcopenia using the mean value of time series data of the vertical displacement of the toe of one foot in the first period and each of the mean values of time series data of the angle of the ankle joint of one foot in each of the second period, the third period, the fourth period, and the fifth period.

The sarcopenia determination unit 113 determines which of a sarcopenia subject, a pre-sarcopenia subject, and a healthy subject the subject is by using the mean value of time series data of the vertical displacement of the toe of one foot in a period of 1% to 50% of one walking cycle, the mean value of time series data of the angle of one ankle joint in a period of 1% to 10% of one walking cycle, the mean value of time series data of the angle of one ankle joint in a period of 21% to 60% of one walking cycle, the mean value of time series data of the angle of one ankle joint in a period of 71% to 80% of one walking cycle, and the mean value of time series data of the angle of one ankle joint in a period of 91% to 100% of one walking cycle.

By inputting the mean value of time series data of the vertical displacement of the toe of one foot in a period of 1% to 50% of one walking cycle, the mean value of time series data of the angle of one ankle joint in a period of 1% to 10% of one walking cycle, the mean value of time series data of the angle of one ankle joint in a period of 21% to 60% of one walking cycle, the mean value of time series data of the angle of one ankle joint in a period of 71% to 80% of one walking cycle, and the mean value of time series data of the angle of one ankle joint in a period of 91% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining sarcopenia by the prediction model created using the vertical displacement of the toe of one foot in the stance phase in isolation was 0.636, the AUC value obtained as a result of determining sarcopenia by the prediction model created using the angle of the ankle joint in the stance phase in isolation was 0.498, and the AUC value obtained as a result of determining sarcopenia by the prediction model created using the angle of the ankle joint in the swing phase in isolation was 0.389. On the other hand, the AUC value obtained as a result of determining sarcopenia by the prediction model created using the vertical displacement of the toe of one foot in the stance phase, the angle of the ankle joint of one foot in the stance phase, and the angle of the ankle joint of one foot in the swing phase was 0.787.

Accordingly, it is possible to determine sarcopenia more accurately in a prediction model created using a vertical displacement of the toe of one foot in the stance phase, an angle of the ankle joint in the stance phase, and an angle of the ankle joint in the swing phase than in a prediction model created using each of the vertical displacement of the toe of one foot in the stance phase, the angle of the ankle joint in the stance phase, and the angle of the ankle joint in the swing phase in isolation.

Subsequently, the walking parameters in the fourteenth modification of the present embodiment will be described.

The walking parameter in the fourteenth modification of the present embodiment may be a mean value of time series data of the vertical displacement of the toe of one foot in the first period of the stance phase of one leg, a mean value of time series data of the vertical displacement of the toe of one foot in the second period of the swing phase of one leg, a mean value of time series data of the angle of the ankle joint of one foot in the third period of the stance phase of one leg, a mean value of time series data of the angle of the ankle joint of one foot in the fourth period of the stance phase and the swing phase of one leg, and a mean value of time series data of the angle of the ankle joint of one foot in the fifth period of the swing phase of one leg.

In the fourteenth modification of the present embodiment, similar to the above experiment, time series data of the vertical displacement of the toe of one foot of each of the plurality of subjects and time series data of the angle of the ankle joint of one foot of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the vertical displacements of the toe of one foot and the mean value of the angles of the ankle joint of one foot in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the vertical displacement of the toe of one foot in the first period of the stance phase, the mean value of time series data of the vertical displacement of the toe of one foot in the second period of the swing phase, the mean value of time series data of the angle of the ankle joint of one foot in the third period of the stance phase, the mean value of time series data of the angle of the ankle joint of one foot in the fourth period of the stance phase and the swing phase, and the mean value of time series data of the angle of the ankle joint of one foot in the fifth period of the swing phase as explanatory variables. The first period is a period of 1% to 50% of one walking cycle, the second period is a period of 71% to 100% of one walking cycle, the third period is a period of 1% to 10% of one walking cycle, the fourth period is a period of 51% to 70% of one walking cycle, and the fifth period is a period of 81% to 100% of one walking cycle. The prediction model was evaluated by cross validation. Leave-one-out cross validation was adopted as the cross validation. Then, the ROC curve of the prediction model in which a healthy subject and a pre-sarcopenia subject were determined was calculated. Furthermore, the AUC value of the ROC curve of the prediction model was calculated.

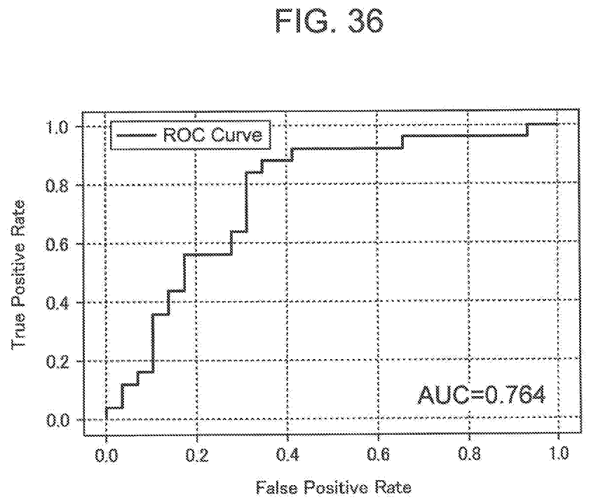

FIG. 36 is a view showing an ROC curve obtained as a result of determining a healthy subject and a pre-sarcopenia subject using the prediction model in the fourteenth modification of the present embodiment.

The prediction model in the fourteenth modification of the present embodiment was created with which of a sarcopenia subject, a pre-sarcopenia subject, and a healthy subject the subject is as an objective variable, and with each of the mean values of the vertical displacements of the toe of one foot in each of periods of 1% to 50% and 71% to 100% of one walking cycle and each of the mean values of the angles of one ankle joint in each of periods of 1% to 10%, 51% to 70%, and 81% to 100% of one walking cycle as explanatory variables. In FIG. 36, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 36 was obtained by plotting the true positive rate and the false positive rate of the prediction model created with the mean value of the vertical displacements of the toe of one foot in a period of 1% to 50% of one walking cycle, the mean value of the vertical displacements of the toe of one foot in a period of 71% to 100% of one walking cycle, the mean value of the angles of one ankle joint in a period of 1% to 10% of one walking cycle, the mean value of the angles of one ankle joint in a period of 51% to 70% of one walking cycle, and the mean value of the angles of one ankle joint in a period of 81% to 100% of one walking cycle as explanatory variables. The AUC value of the ROC curve shown in FIG. 36 was 0.764.

In the fourteenth modification of the present embodiment, the mean value of the vertical displacements of the toe of one foot in a period of 1% to 50% of one walking cycle, the mean value of the vertical displacements of the toe of one foot in a period of 71% to 100% of one walking cycle, the mean value of the angles of one ankle joint in a period of 1% to 10% of one walking cycle, the mean value of the angles of one ankle joint in a period of 51% to 70% of one walking cycle, and the mean value of the angles of one ankle joint in a period of 81% to 100% of one walking cycle are determined as walking parameters. In addition, the prediction model created with the mean value of the vertical displacements of the toe of one foot in a period of 1% to 50% of one walking cycle, the mean value of the vertical displacements of the toe of one foot in a period of 71% to 100% of one walking cycle, the mean value of the angles of one ankle joint in a period of 1% to 10% of one walking cycle, the mean value of the angles of one ankle joint in a period of 51% to 70% of one walking cycle, and the mean value of the angles of one ankle joint in a period of 81% to 100% of one walking cycle as explanatory variables is determined as a prediction model to be used by the sarcopenia determination unit 113.

The walking parameter detection unit 112 detects time series data of the vertical displacement of the toe of one foot in the first period of the stance phase, time series data of the vertical displacement of the toe of one foot in the second period of the swing phase, time series data of the angle of the ankle joint of one foot in the third period of the stance phase, time series data of the angle of the ankle joint of one foot in the fourth period of the stance phase and the swing phase, and time series data of the angle of the ankle joint of one foot in the fifth period of the swing phase. The first period is a period of 1% to 50% of one walking cycle, the second period is a period of 71% to 100% of one walking cycle, the third period is a period of 1% to 10% of one walking cycle, the fourth period is a period of 51% to 70% of one walking cycle, and the fifth period is a period of 81% to 100% of one walking cycle.

The walking parameter detection unit 112 detects time series data of the vertical displacement of the toe of one foot in a period of 1% to 50% of one walking cycle, time series data of the vertical displacement of the toe of one foot in a period of 71% to 100% of one walking cycle, time series data of the angle of one ankle joint in a period of 1% to 10% of one walking cycle, time series data of the angle of one ankle joint in a period of 51% to 70% of one walking cycle, and time series data of the angle of one ankle joint in a period of 81% to 100% of one walking cycle.

In addition, the walking parameter detection unit 112 calculates the mean value of time series data of the vertical displacement of the toe of one foot in a period of 1% to 50% of one walking cycle, the mean value of time series data of the vertical displacement of the toe of one foot in a period of 71% to 100% of one walking cycle, the mean value of time series data of the angle of one ankle joint in a period of 1% to 10% of one walking cycle, the mean value of time series data of the angle of one ankle joint in a period of 51% to 70% of one walking cycle, and the mean value of time series data of the angle of one ankle joint in a period of 81% to 100% of one walking cycle.

The memory 12 stores in advance a prediction model generated with the mean value of time series data of the vertical displacement of the toe of one foot in the first period of the stance phase, the mean value of time series data of the vertical displacement of the toe of the one foot in the second period of the swing phase, the mean value of time series data of the angle of the ankle joint of the one foot in the third period of the stance phase, the mean value of time series data of the angle of the ankle joint of the one foot in the fourth period of the stance phase and the swing phase, and the mean value of time series data of the angle of the ankle joint of the one foot in the fifth period of the swing phase as input values and with which of a sarcopenia subject, a pre-sarcopenia subject, and a healthy subject the subject is as an output value.

The memory 12 stores in advance a prediction model generated with the mean value of time series data of the vertical displacement of the toe of one foot in a period of 1% to 50% of one walking cycle, the mean value of time series data of the vertical displacement of the toe of the one foot in a period of 71% to 100% of one walking cycle, the mean value of time series data of the angle of one ankle joint in a period of 1% to 10% of one walking cycle, the mean value of time series data of the angle of one ankle joint in a period of 51% to 70% of one walking cycle, and the mean value of time series data of the angle of one ankle joint in a period of 81% to 100% of one walking cycle as input values and with which of a sarcopenia subject, a pre-sarcopenia subject, and a healthy subject the subject is as an output value.

The sarcopenia determination unit 113 determines whether or not the subject has sarcopenia using the each of mean values of time series data of the vertical displacement of the toe of one foot in each of the first period and the second period, and each of the mean values of time series data of the angle of the ankle joint of one foot in each of the third period, the fourth period, and the fifth period.

The sarcopenia determination unit 113 determines which of a sarcopenia subject, a pre-sarcopenia subject, and a healthy subject the subject is by using the mean value of time series data of the vertical displacement of the toe of one foot in a period of 1% to 50% of one walking cycle, the mean value of time series data of the vertical displacement of the toe of one foot in a period of 71% to 100% of one walking cycle, the mean value of time series data of the angle of one ankle joint in a period of 1% to 10% of one walking cycle, the mean value of time series data of the angle of one ankle joint in a period of 51% to 70% of one walking cycle, and the mean value of time series data of the angle of one ankle joint in a period of 81% to 100% of one walking cycle.

By inputting the mean value of time series data of the vertical displacement of the toe of one foot in a period of 1% to 50% of one walking cycle, the mean value of time series data of the vertical displacement of the toe of one foot in a period of 71% to 100% of one walking cycle, the mean value of time series data of the angle of one ankle joint in a period of 1% to 10% of one walking cycle, the mean value of time series data of the angle of one ankle joint in a period of 51% to 70% of one walking cycle, and the mean value of time series data of the angle of one ankle joint in a period of 81% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining a pre-sarcopenia subject by the prediction model created using the vertical displacement of the toe of one foot in the stance phase in isolation was 0.560, the AUC value obtained as a result of determining a pre-sarcopenia subject by the prediction model created using the vertical displacement of the toe of one foot in the swing phase in isolation was 0.626, the AUC value obtained as a result of determining a pre-sarcopenia subject by the prediction model created using the angle of the ankle joint in the stance phase in isolation was 0.610, and the AUC value obtained as a result of determining a pre-sarcopenia subject by the prediction model created using the angle of the ankle joint in the swing phase in isolation was 0.622. On the other hand, the AUC value obtained as a result of determining a pre-sarcopenia subject by the prediction model created using the vertical displacement of the toe of one foot in the stance phase, the vertical displacement of the toe of one foot in the swing phase, the angle of the ankle joint of one foot in the stance phase, and the angle of the ankle joint of one foot in the swing phase was 0.764.

Accordingly, it is possible to determine a pre-sarcopenia subject more accurately in a prediction model created using a vertical displacement of the toe of one foot in the stance phase, a vertical displacement of the toe of one foot in the swing phase, an angle of the ankle joint in the stance phase, and an angle of the ankle joint in the swing phase than in a prediction model created using each of the vertical displacement of the toe of one foot in the stance phase, the vertical displacement of the toe of one foot in the swing phase, the angle of the ankle joint in the stance phase, and the angle of the ankle joint in the swing phase in isolation.

Subsequently, the walking parameter in the fifteenth modification of the present embodiment will be described.

The walking parameter in the fifteenth modification of the present embodiment may be a mean value of time series data of the angle of the knee joint of one leg in the first period of the stance phase of one leg, a mean value of time series data of the angle of the knee joint of one leg in the second period of the swing phase of one leg, a mean value of time series data of the angle of the knee joint of one leg in the third period of the swing phase of one leg, a mean value of time series data of the angle of the ankle joint of one foot in the fourth period of the stance phase of one leg, a mean value of time series data of the angle of the ankle joint of one foot in the fifth period of the swing phase of one leg, and a mean value of time series data of the angle of the ankle joint of one foot in the sixth period of the swing phase of one leg.

In the fifteenth modification of the present embodiment, similar to the above experiment, time series data of the angle of the knee joint of one leg of each of the plurality of subjects and time series data of the angle of the ankle joint of one foot of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the angles of the knee joint of one leg and the mean value of the angles of the ankle joint of one foot in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the angle of the knee joint of one leg in the first period of the stance phase, the mean value of time series data of the angle of the knee joint of one leg in the second period of the swing phase, the mean value of time series data of the angle of the knee joint of one leg in the third period of the swing phase, the mean value of time series data of the angle of the ankle joint of one foot in the fourth period of the stance phase, the mean value of time series data of the angle of the ankle joint of one foot in the fifth period of the swing phase, and the mean value of time series data of the angle of the ankle joint of one foot in the sixth period of the swing phase as explanatory variables. The first period is a period of 1% to 40% of one walking cycle, the second period is a period of 61% to 70% of one walking cycle, the third period is a period of 81% to 100% of one walking cycle, the fourth period is a period of 1% to 50% of one walking cycle, the fifth period is a period of 61% to 70% of one walking cycle, and the sixth period is a period of 91% to 100% of one walking cycle. The prediction model was evaluated by cross validation. Leave-one-out cross validation was adopted as the cross validation. Then, the ROC curve of the prediction model in which a healthy subject and a sarcopenia subject were determined was calculated. Furthermore, the AUC value of the ROC curve of the prediction model was calculated.

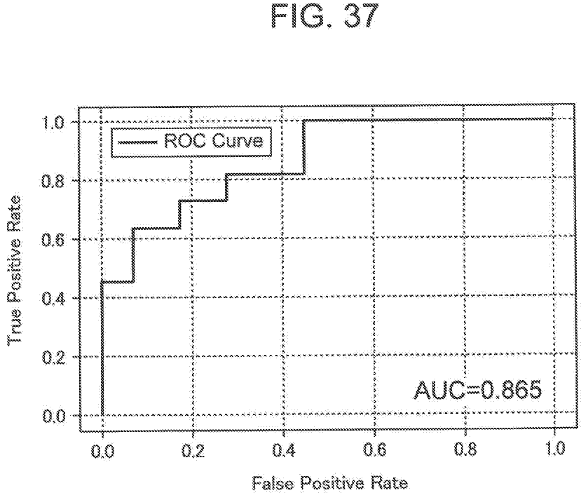

FIG. 37 is a view showing an ROC curve obtained as a result of determining a healthy subject and a sarcopenia subject using a prediction model in the fifteenth modification of the present embodiment.

The prediction model in the fifteenth modification of the present embodiment was created with which of a sarcopenia subject, a pre-sarcopenia subject, and a healthy subject the subject is as an objective variable, and with each of the mean values of the angles of one knee joint in each of periods of 1% to 40%, 61% to 70%, and 81% to 100% of one walking cycle and each of the mean values of the angles of one ankle joint in each of periods of 1% to 50%, 61% to 70%, and 91% to 100% of one walking cycle as explanatory variables. In FIG. 37, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 37 was obtained by plotting the true positive rate and the false positive rate of the prediction model created with the mean value of the angles of the knee joint of one leg in a period of 1% to 40% of one walking cycle, the mean value of the angles of the knee joint of one leg in a period of 61% to 70% of one walking cycle, the mean value of the angles of the knee joint of one leg in a period of 81% to 100% of one walking cycle, the mean value of the angles of one ankle joint in a period of 1% to 50% of one walking cycle, the mean value of the angles of one ankle joint in a period of 61% to 70% of one walking cycle, and the mean value of the angles of one ankle joint in a period of 91% to 100% of one walking cycle as explanatory variables. The AUC value of the ROC curve shown in FIG. 37 was 0.865.

In the fifteenth modification of the present embodiment, the mean value of the angles of the knee joint of one leg in a period of 1% to 40% of one walking cycle, the mean value of the angles of the knee joint of one leg in a period of 61% to 70% of one walking cycle, the mean value of the angles of the knee joint of one leg in a period of 81% to 100% of one walking cycle, the mean value of the angles of one ankle joint in a period of 1% to 50% of one walking cycle, the mean value of the angles of one ankle joint in a period of 61% to 70% of one walking cycle, and the mean value of the angles of one ankle joint in a period of 91% to 100% of one walking cycle are determined as walking parameters. In addition, the prediction model created with the mean value of the angles of the knee joint of one leg in a period of 1% to 40% of one walking cycle, the mean value of the angles of the knee joint of one leg in a period of 61% to 70% of one walking cycle, the mean value of the angles of the knee joint of one leg in a period of 81% to 100% of one walking cycle, the mean value of the angles of one ankle joint in a period of 1% to 50% of one walking cycle, the mean value of the angles of one ankle joint in a period of 61% to 70% of one walking cycle, and the mean value of the angles of one ankle joint in a period of 91% to 100% of one walking cycle as explanatory variables is determined as a prediction model to be used by the sarcopenia determination unit 113.

The walking parameter detection unit 112 detects time series data of the angle of the knee joint of one leg in the first period of the stance phase, time series data of the angle of the knee joint of one leg in the second period of the swing phase, time series data of the angle of the knee joint of one leg in the third period of the swing phase, time series data of the angle of the ankle joint of one foot in the fourth period of the stance phase, time series data of the angle of the ankle joint of one foot in the fifth period of the swing phase, and time series data of the angle of the ankle joint of one foot in the sixth period of the swing phase. The first period is a period of 1% to 40% of one walking cycle, the second period is a period of 61% to 70% of one walking cycle, the third period is a period of 81% to 100% of one walking cycle, the fourth period is a period of 1% to 50% of one walking cycle, the fifth period is a period of 61% to 70% of one walking cycle, and the sixth period is a period of 91% to 100% of one walking cycle.

The walking parameter detection unit 112 detects time series data of the angle of the knee joint of one leg in a period of 1% to 40% of one walking cycle, time series data of the angle of the knee joint of one leg in a period of 61% to 70% of one walking cycle, time series data of the angle of the knee joint of one leg in a period of 81% to 100% of one walking cycle, time series data of the angle of one ankle joint in a period of 1% to 50% of one walking cycle, time series data of the angle of one ankle joint in a period of 61% to 70% of one walking cycle, and time series data of the angle of one ankle joint in a period of 91% to 100% of one walking cycle.

In addition, the walking parameter detection unit 112 calculates the mean value of time series data of the angle of the knee joint of one leg in a period of 1% to 40% of one walking cycle, the mean value of time series data of the angle of the knee joint of one leg in a period of 61% to 70% of one walking cycle, the mean value of time series data of the angle of the knee joint of one leg in a period of 81% to 100% of one walking cycle, the mean value of time series data of the angle of one ankle joint in a period of 1% to 50% of one walking cycle, the mean value of time series data of the angle of one ankle joint in a period of 61% to 70% of one walking cycle, and the mean value of time series data of the angle of one ankle joint in a period of 91% to 100% of one walking cycle.

The memory 12 stores in advance a prediction model generated with the mean value of time series data of the angle of the knee joint of one leg in the first period of the stance phase, the mean value of time series data of the angle of the knee joint of the one leg in the second period of the swing phase, the mean value of time series data of the angle of the knee joint of the one leg in the third period of the swing phase, the mean value of time series data of the angle of the ankle joint of one foot in the fourth period of the stance phase, the mean value of time series data of the angle of the ankle joint of the one foot in the fifth period of the swing phase, and the mean value of time series data of the angle of the ankle joint of the one foot in the sixth period of the swing phase as input values and with which of a sarcopenia subject, a pre-sarcopenia subject, and a healthy subject the subject is as an output value.

The memory 12 stores in advance a prediction model generated with the mean value of time series data of the angle of the knee joint of one leg in a period of 1% to 40% of one walking cycle, the mean value of time series data of the angle of the knee joint of the one leg in a period of 61% to 70% of one walking cycle, the mean value of time series data of the angle of the knee joint of the one leg in a period of 81% to 100% of one walking cycle, the mean value of time series data of the angle of one ankle joint in a period of 1% to 50% of one walking cycle, the mean value of time series data of the angle of one ankle joint in a period of 61% to 70% of one walking cycle, and the mean value of time series data of the angle of one ankle joint in a period of 91% to 100% of one walking cycle as input values and with which of a sarcopenia subject, a pre-sarcopenia subject, and a healthy subject the subject is as an output value.

The sarcopenia determination unit 113 determines whether or not the subject has sarcopenia using each of the means value of time series data of the angle of the knee joint of one leg in each of the first period, the second period, and the third period, and each of the mean values of time series data of the angle of the ankle joint of one foot in each of the fourth period, the fifth period, and the sixth period.

The sarcopenia determination unit 113 determines which of a sarcopenia subject, a pre-sarcopenia subject, and a healthy subject the subject is by using the mean value of time series data of the angle of the knee joint of one leg in a period of 1% to 40% of one walking cycle, the mean value of time series data of the angle of the knee joint of one leg in a period of 61% to 70% of one walking cycle, the mean value of time series data of the angle of the knee joint of one leg in a period of 81% to 100% of one walking cycle, the mean value of time series data of the angle of one ankle joint in a period of 1% to 50% of one walking cycle, the mean value of time series data of the angle of one ankle joint in a period of 61% to 70% of one walking cycle, and the mean value of time series data of the angle of one ankle joint in a period of 91% to 100% of one walking cycle.

By inputting the mean value of time series data of the angle of the knee joint of one leg in a period of 1% to 40% of one walking cycle, the mean value of time series data of the angle of the knee joint of the one leg in a period of 61% to 70% of one walking cycle, the mean value of time series data of the angle of one ankle joint in a period of 81% to 100% of one walking cycle, the mean value of time series data of the angle of one ankle joint in a period of 1% to 50% of one walking cycle, the mean value of time series data of the angle of one ankle joint in a period of 61% to 70% of one walking cycle, and the mean value of time series data of the angle of one ankle joint in a period of 91% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining sarcopenia by the prediction model created using the angle of the knee joint of one leg in the stance phase in isolation was 0.586, the AUC value obtained as a result of determining sarcopenia by the prediction model created using the angle of the knee joint of one leg in the swing phase in isolation was 0.699, the AUC value obtained as a result of determining sarcopenia by the prediction model created using the angle of the ankle joint in the stance phase in isolation was 0.498, and the AUC value obtained as a result of determining sarcopenia by the prediction model created using the angle of the ankle joint in the swing phase in isolation was 0.389. On the other hand, the AUC value obtained as a result of determining sarcopenia by the prediction model created using the angle of the knee joint of one leg in the stance phase, the angle of the knee joint of one leg in the swing phase, the angle of the ankle joint of one foot in the stance phase, and the angle of the ankle joint of one foot in the swing phase was 0.865.

Accordingly, it is possible to determine sarcopenia more accurately in a prediction model created using an angle of the knee joint of one leg in the stance phase, an angle of the knee joint of one leg in the swing phase, an angle of the ankle joint in the stance phase, and an angle of the ankle joint in the swing phase than in a prediction model created using each of the angle of the knee joint of one leg in the stance phase, the angle of the knee joint of one leg in the swing phase, the angle of the ankle joint in the stance phase, and the angle of the ankle joint in the swing phase in isolation.

Subsequently, the walking parameters in the sixteenth modification of the present embodiment will be described.

The walking parameter in the sixteenth modification of the present embodiment may be a mean value of time series data of the angle of the knee joint of one leg in the first period of the stance phase of one leg, a mean value of time series data of the angle of the knee joint of one leg in the second period of the swing phase of one leg, a mean value of time series data of the angle of the ankle joint of one foot in the third period of the stance phase of one leg, and a mean value of time series data of the angle of the ankle joint of one foot in the fourth period of the stance phase and the swing phase of one leg.

In the sixteenth modification of the present embodiment, similar to the above experiment, time series data of the angle of the knee joint of one leg of each of the plurality of subjects and time series data of the angle of the ankle joint of one foot of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the angles of the knee joint of one leg and the mean value of the angles of the ankle joint of one foot in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the angle of the knee joint of one leg in the first period of the stance phase, the mean value of time series data of the angle of the knee joint of one leg in the second period of the swing phase, the mean value of time series data of the angle of the ankle joint of one foot in the third period of the stance phase, and the mean value of time series data of the angle of the ankle joint of one foot in the fourth period of the stance phase and the swing phase as explanatory variables. The first period is a period of 1% to 10% of one walking cycle, the second period is a period of 81% to 100% of one walking cycle, the third period is a period of 1% to 10% of one walking cycle, and the fourth period is a period of 21% to 70% of one walking cycle. The prediction model was evaluated by cross validation. Leave-one-out cross validation was adopted as the cross validation. Then, the ROC curve of the prediction model in which a healthy subject and a pre-sarcopenia subject were determined was calculated. Furthermore, the AUC value of the ROC curve of the prediction model was calculated.

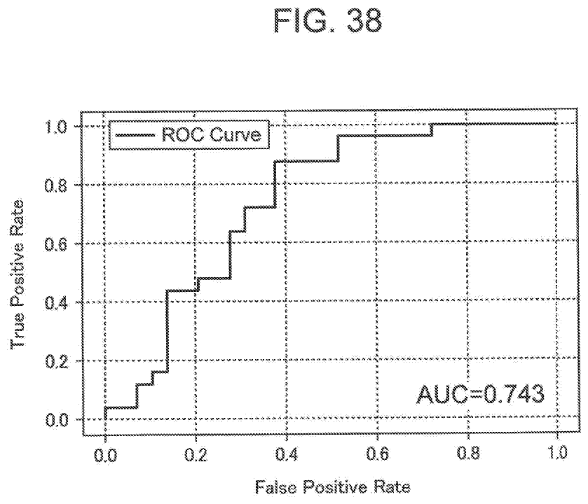

FIG. 38 is a view showing an ROC curve obtained as a result of determining a healthy subject and a pre-sarcopenia subject using the prediction model in the sixteenth modification of the present embodiment.

The prediction model in the sixteenth modification of the present embodiment was created with which of a sarcopenia subject, a pre-sarcopenia subject, and a healthy subject the subject is as an objective variable, and with each of the mean values of the angles of one knee joint in each of periods of 1% to 10% and 81% to 100% of one walking cycle and each of the mean values of the angles of one ankle joint in each of periods of 1% to 10% and 21% to 70% of one walking cycle as explanatory variables. In FIG. 38, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 38 was obtained by plotting the true positive rate and the false positive rate of the prediction model created with the mean value of the angles of the knee joint of one leg in a period of 1% to 10% of one walking cycle, the mean value of the angles of the knee joint of one leg in a period of 81% to 100% of one walking cycle, the mean value of the angles of one ankle joint in a period of 1% to 10% of one walking cycle, and the mean value of the angles of one ankle joint in a period of 21% to 70% of one walking cycle as explanatory variables. The AUC value of the ROC curve shown in FIG. 38 was 0.743.

In the sixteenth modification of the present embodiment, the mean value of the angles of the knee joint of one leg in a period of 1% to 10% of one walking cycle, the mean value of the angles of the knee joint of one leg in a period of 81% to 100% of one walking cycle, the mean value of the angles of one ankle joint in a period of 1% to 10% of one walking cycle, and the mean value of the angles of one ankle joint in a period of 21% to 70% of one walking cycle are determined as walking parameters. In addition, the prediction model created with the mean value of the angles of the knee joint of one leg in a period of 1% to 10% of one walking cycle, the mean value of the angles of the knee joint of one leg in a period of 81% to 100% of one walking cycle, the mean value of the angles of one ankle joint in a period of 1% to 10% of one walking cycle, and the mean value of the angles of one ankle joint in a period of 21% to 70% of one walking cycle as explanatory variables is determined as a prediction model to be used by the sarcopenia determination unit 113.

The walking parameter detection unit 112 detects time series data of the angle of the knee joint of one leg in the first period of the stance phase, time series data of the angle of the knee joint of one leg in the second period of the swing phase, time series data of the angle of the ankle joint of one foot in the third period of the stance phase, and time series data of the angle of the ankle joint of one foot in the fourth period of the stance phase and the swing phase. The first period is a period of 1% to 10% of one walking cycle, the second period is a period of 81% to 100% of one walking cycle, the third period is a period of 1% to 10% of one walking cycle, and the fourth period is a period of 21% to 70% of one walking cycle.

The walking parameter detection unit 112 detects time series data of the angle of the knee joint of one leg in a period of 1% to 10% of one walking cycle, time series data of the angle of the knee joint of one leg in a period of 81% to 100% of one walking cycle, time series data of the angle of one ankle joint in a period of 1% to 10% of one walking cycle, and time series data of the angle of one ankle joint in a period of 21% to 70% of one walking cycle.

In addition, the walking parameter detection unit 112 calculates the mean value of time series data of the angle of the knee joint of one leg in a period of 1% to 10% of one walking cycle, the mean value of time series data of the angle of the knee joint of one leg in a period of 81% to 100% of one walking cycle, the mean value of time series data of the angle of one ankle joint in a period of 1% to 10% of one walking cycle, and the mean value of time series data of the angle of one ankle joint in a period of 21% to 70% of one walking cycle.

The memory 12 stores in advance a prediction model generated with the mean value of time series data of the angle of the knee joint of one leg in the first period of the stance phase, the mean value of time series data of the angle of the knee joint of the one leg in the second period of the swing phase, the mean value of time series data of the angle of the ankle joint of one foot in the third period of the stance phase, and the mean value of time series data of the angle of the ankle joint of the one foot in the fourth period of the stance phase and the swing phase as input values and with which of a sarcopenia subject, a pre-sarcopenia subject, and a healthy subject the subject is as an output value.

The memory 12 stores in advance a prediction model generated with the mean value of time series data of the angle of the knee joint of one leg in a period of 1% to 10% of one walking cycle, the mean value of time series data of the angle of the knee joint of the one leg in a period of 81% to 100% of one walking cycle, the mean value of time series data of the angle of one ankle joint in a period of 1% to 10% of one walking cycle, and the mean value of time series data of the angle of one ankle joint in a period of 21% to 70% of one walking cycle as input values and with which of a sarcopenia subject, a pre-sarcopenia subject, and a healthy subject the subject is as an output value.

The sarcopenia determination unit 113 determines whether or not the subject has sarcopenia using each of the mean values of time series data of the angle of the knee joint of one leg in each of the first period and the second period, and each of the mean values of time series data of the angle of the ankle joint of one foot in each of the third period and the fourth period.

The sarcopenia determination unit 113 determines which of a sarcopenia subject, a pre-sarcopenia subject, and a healthy subject the subject is by using the mean value of time series data of the angle of the knee joint of one leg in a period of 1% to 10% of one walking cycle, the mean value of time series data of the angle of the knee joint of one leg in a period of 81% to 100% of one walking cycle, the mean value of time series data of the angle of one ankle joint in a period of 1% to 10% of one walking cycle, and the mean value of time series data of the angle of one ankle joint in a period of 21% to 70% of one walking cycle.

By inputting the mean value of time series data of the angle of the knee joint of one leg in a period of 1% to 10% of one walking cycle, the mean value of time series data of the angle of the knee joint of the one leg in a period of 81% to 100% of one walking cycle, the mean value of time series data of the angle of one ankle joint in a period of 1% to 10% of one walking cycle, and the mean value of time series data of the angle of one ankle joint in a period of 21% to 70% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining a pre-sarcopenia subject by the prediction model created using the angle of the knee joint of one leg in the stance phase in isolation was 0.537, the AUC value obtained as a result of determining a pre-sarcopenia subject by the prediction model created using the angle of the knee joint of one leg in the swing phase in isolation was 0.604, the AUC value obtained as a result of determining a pre-sarcopenia subject by the prediction model created using the angle of the ankle joint in the stance phase in isolation was 0.610, and the AUC value obtained as a result of determining a pre-sarcopenia subject by the prediction model created using the angle of the ankle joint in the swing phase in isolation was 0.622. On the other hand, the AUC value obtained as a result of determining a pre-sarcopenia subject by the prediction model created using the angle of the knee joint of one leg in the stance phase, the angle of the knee joint of one leg in the swing phase, the angle of the ankle joint of one foot in the stance phase, and the angle of the ankle joint of one foot in the swing phase was 0.743.

Accordingly, it is possible to determine a pre-sarcopenia subject more accurately in a prediction model created using an angle of the knee joint of one leg in the stance phase, an angle of the knee joint of one leg in the swing phase, an angle of the ankle joint in the stance phase, and an angle of the ankle joint in the swing phase than in a prediction model created using each of the angle of the knee joint of one leg in the stance phase, the angle of the knee joint of one leg in the swing phase, the angle of the ankle joint in the stance phase, and the angle of the ankle joint in the swing phase in isolation.

Subsequently, the walking parameters in the seventeenth modification of the present embodiment will be described.

The walking parameter in the seventeenth modification of the present embodiment may be a mean value of time series data of the vertical displacement of the toe of one foot in the first period of the stance phase of one leg, a mean value of time series data of the vertical displacement of the toe of one foot in the second period of the stance phase of one leg, a mean value of time series data of the vertical displacement of the toe of one foot in the third period of the swing phase of one leg, a mean value of time series data of the angle of the knee joint of one leg in the fourth period of the stance phase of one leg, a mean value of time series data of the angle of the knee joint of one leg in the fifth period of the stance phase and the swing phase of one leg, a mean value of time series data of the angle of the ankle joint of one foot in the sixth period of the stance phase of one leg, and a mean value of time series data of the angle of the ankle joint of one foot in the seventh period of the stance phase and the swing phase of one leg.

In the seventeenth modification of the present embodiment, similar to the above experiment, time series data of the vertical displacement of the toe of one foot of each of the plurality of subjects, time series data of the angle of the knee joint of one leg of each of the plurality of subjects, and time series data of the angle of the ankle joint of one foot of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the vertical displacements of the toe of one foot, the mean value of the angles of the knee joint of one leg, and the mean value of the angles of the ankle joint of one foot in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the vertical displacement of the toe of one foot in the first period of the stance phase, the mean value of time series data of the vertical displacement of the toe of one foot in the second period of the stance phase, the mean value of time series data of the vertical displacement of the toe of one foot in the third period of the swing phase, the mean value of time series data of the angle of the knee joint of one leg in the fourth period of the stance phase, the mean value of time series data of the angle of the knee joint of one leg in the fifth period of the stance phase and the swing phase, the mean value of time series data of the angle of the ankle joint of one foot in the sixth period of the stance phase, and the mean value of time series data of the angle of the ankle joint of one foot in the seventh period of the stance phase and the swing phase as explanatory variables. The first period is a period of 21% to 30% of one walking cycle, the second period is a period of 51% to 60% of one walking cycle, the third period is a period of 81% to 100% of one walking cycle, the fourth period is a period of 11% to 20% of one walking cycle, the fifth period is a period of 41% to 80% of one walking cycle, the sixth period is a period of 1% to 20% of one walking cycle, and the seventh period is a period of 51% to 80% of one walking cycle. The prediction model was evaluated by cross validation. Leave-one-out cross validation was adopted as the cross validation. Then, the ROC curve of the prediction model in which a healthy subject and a sarcopenia subject were determined was calculated. Furthermore, the AUC value of the ROC curve of the prediction model was calculated.

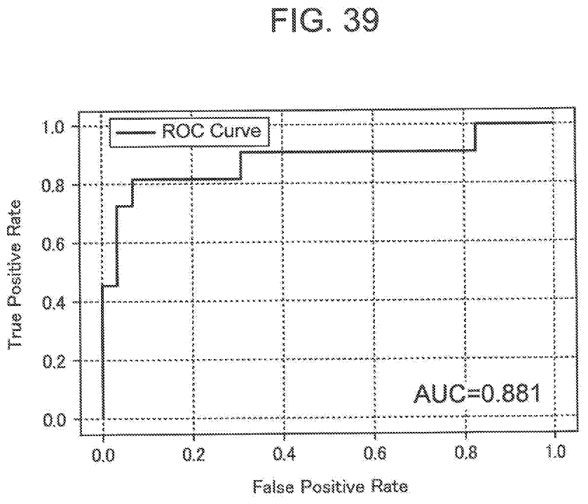

FIG. 39 is a view showing an ROC curve obtained as a result of determining a healthy subject and a sarcopenia subject using a prediction model in the seventeenth modification of the present embodiment.

The prediction model in the seventeenth modification of the present embodiment was created with which of a sarcopenia subject, a pre-sarcopenia subject, and a healthy subject the subject is as an objective variable, and with each of the mean values of the vertical displacements of the toe of one foot in each of periods of 21% to 30%, 51% to 60%, and 81% to 100% of one walking cycle, each of the mean values of the angles of one knee joint in each of periods of 11% to 20% and 41% to 80% of one walking cycle, and each of the mean values of the angles of one ankle joint in each of periods of 1% to 20% and 51% to 80% of one walking cycle as explanatory variables. In FIG. 39, the vertical axis represents the true positive rate, and the horizontal axis represents the false positive rate. The true positive rate indicates a ratio at which the prediction model has correctly determined a sarcopenia subject as having sarcopenia, and the false positive rate indicates a ratio at which the prediction model has incorrectly determined a healthy subject as having sarcopenia.

The ROC curve shown in FIG. 39 was obtained by plotting the true positive rate and the false positive rate of the prediction model created with the mean value of the vertical displacements of the toe of one foot in a period of 21% to 30% of one walking cycle, the mean value of the vertical displacements of the toe of one foot in a period of 51% to 60% of one walking cycle, the mean value of the vertical displacements of the toe of one foot in a period of 81% to 100% of one walking cycle, the mean value of the angles of one knee joint in a period of 11% to 20% of one walking cycle, the mean value of the angles of one knee joint in a period of 41% to 80% of one walking cycle, the mean value of the angles of one ankle joint in a period of 1% to 20% of one walking cycle, and the mean value of the angles of one ankle joint in a period of 51% to 80% of one walking cycle as explanatory variables. The AUC value of the ROC curve shown in FIG. 39 was 0.881.

In the seventeenth modification of the present embodiment, the mean value of the vertical displacements of the toe of one foot in a period of 21% to 30% of one walking cycle, the mean value of the vertical displacements of the toe of one foot in a period of 51% to 60% of one walking cycle, the mean value of the vertical displacements of the toe of one foot in a period of 81% to 100% of one walking cycle, the mean value of the angles of one knee joint in a period of 11% to 20% of one walking cycle, the mean value of the angles of one knee joint in a period of 41% to 80% of one walking cycle, the mean value of the angles of one ankle joint in a period of 1% to 20% of one walking cycle, and the mean value of the angles of one ankle joint in a period of 51% to 80% of one walking cycle are determined as walking parameters.

In addition, the prediction model created with the mean value of the vertical displacements of the toe of one foot in a period of 21% to 30% of one walking cycle, the mean value of the vertical displacements of the toe of one foot in a period of 51% to 60% of one walking cycle, the mean value of the vertical displacements of the toe of one foot in a period of 81% to 100% of one walking cycle, the mean value of the angles of one knee joint in a period of 11% to 20% of one walking cycle, the mean value of the angles of one knee joint in a period of 41% to 80% of one walking cycle, the mean value of the angles of one ankle joint in a period of 1% to 20% of one walking cycle, and the mean value of the angles of one ankle joint in a period of 51% to 80% of one walking cycle as explanatory variables is determined as a prediction model to be used by the sarcopenia determination unit 113.

The walking parameter detection unit 112 detects time series data of the vertical displacement of the toe of one foot in the first period of the stance phase, time series data of the vertical displacement of the toe of one foot in the second period of the stance phase, time series data of the vertical displacement of the toe of one foot in the third period of the swing phase, time series data of the angle of the knee joint of one leg in the fourth period of the stance phase, time series data of the angle of the knee joint of one leg in the fifth period of the stance phase and the swing phase, time series data of the angle of the ankle joint of one foot in the sixth period of the stance phase, and time series data of the angle of the ankle joint of one foot in the seventh period of the stance phase and the swing phase. The first period is a period of 21% to 30% of one walking cycle, the second period is a period of 51% to 60% of one walking cycle, the third period is a period of 81% to 100% of one walking cycle, the fourth period is a period of 11% to 20% of one walking cycle, the fifth period is a period of 41% to 80% of one walking cycle, the sixth period is a period of 1% to 20% of one walking cycle, and the seventh period is a period of 51% to 80% of one walking cycle.

The walking parameter detection unit 112 detects time series data of the vertical displacement of the toe of one foot in a period of 21% to 30% of one walking cycle, time series data of the vertical displacement of the toe of one foot in a period of 51% to 60% of one walking cycle, time series data of the vertical displacement of the toe of one foot in a period of 81% to 100% of one walking cycle, time series data of the angle of one knee joint in a period of 11% to 20% of one walking cycle, time series data of the angle of one knee joint in a period of 41% to 80% of one walking cycle, time series data of the angle of one ankle joint in a period of 1% to 20% of one walking cycle, and time series data of the angle of one ankle joint in a period of 51% to 80% of one walking cycle.

In addition, the walking parameter detection unit 112 calculates the mean value of time series data of the vertical displacement of the toe of one foot in a period of 21% to 30% of one walking cycle, the mean value of time series data of the vertical displacement of the toe of one foot in a period of 51% to 60% of one walking cycle, the mean value of time series data of the vertical displacement of the toe of one foot in a period of 81% to 100% of one walking cycle, the mean value of time series data of the angle of one knee joint in a period of 11% to 20% of one walking cycle, the mean value of time series data of the angle of one knee joint in a period of 41% to 80% of one walking cycle, the mean value of time series data of the angle of one ankle joint in a period of 1% to 20% of one walking cycle, and the mean value of time series data of the angle of one ankle joint in a period of 51% to 80% of one walking cycle.

The memory 12 stores in advance a prediction model generated with the mean value of time series data of the vertical displacement of the toe of one foot in the first period of the stance phase, the mean value of time series data of the vertical displacement of the toe of the one foot in the second period of the stance phase, the mean value of time series data of the vertical displacement of the toe of the one foot in the third period of the swing phase, the mean value of time series data of the angle of the knee joint of one leg in the fourth period of the stance phase, the mean value of time series data of the angle of the knee joint of the one leg in the fifth period of the stance phase and the swing phase, the mean value of time series data of the angle of the ankle joint of one foot in the sixth period of the stance phase, and the mean value of time series data of the angle of the ankle joint of the one foot in the seventh period of the stance phase and the swing phase as input values and with which of a sarcopenia subject, a pre-sarcopenia subject, and a healthy subject the subject is as an output value.

The memory 12 stores in advance a prediction model generated with the mean value of time series data of the vertical displacement of the toe of one foot in a period of 21% to 30% of one walking cycle, the mean value of time series data of the vertical displacement of the toe of the one foot in a period of 51% to 60% of one walking cycle, the mean value of time series data of the vertical displacement of the toe of the one foot in a period of 81% to 100% of one walking cycle, the mean value of time series data of the angle of one knee joint in a period of 11% to 20% of one walking cycle, the mean value of time series data of the angle of the one knee joint in a period of 41% to 80% of one walking cycle, the mean value of time series data of the angle of one ankle joint in a period of 1% to 20% of one walking cycle, and the mean value of time series data of the angle of the one ankle joint in a period of 51% to 80% of one walking cycle as input values and with which of a sarcopenia subject, a pre-sarcopenia subject, and a healthy subject the subject is as an output value.

The sarcopenia determination unit 113 determines whether or not the subject has sarcopenia using each of the mean values of time series data of the vertical displacement of the toe of one foot in each of the first period, the second period, and the third period, each of the mean values of time series data of the angle of the knee joint of one leg in each of the fourth period and the fifth period, and each of the mean values of time series data of the angle of the ankle joint of one foot in each of the sixth period and the seventh period.

The sarcopenia determination unit 113 determines which of a sarcopenia subject, a pre-sarcopenia subject, and a healthy subject the subject is by using the mean value of time series data of the vertical displacement of the toe of one foot in a period of 21% to 30% of one walking cycle, the mean value of time series data of the vertical displacement of the toe of one foot in a period of 51% to 60% of one walking cycle, the mean value of time series data of the vertical displacement of the toe of one foot in a period of 81% to 100% of one walking cycle, the mean value of time series data of the angle of one knee joint in a period of 11% to 20% of one walking cycle, the mean value of time series data of the angle of one knee joint in a period of 41% to 80% of one walking cycle, the mean value of time series data of the angle of one ankle joint in a period of 1% to 20% of one walking cycle, and the mean value of time series data of the angle of one ankle joint in a period of 51% to 80% of one walking cycle.

By inputting the mean value of time series data of the vertical displacement of the toe of one foot in a period of 21% to 30% of one walking cycle, the mean value of time series data of the vertical displacement of the toe of one foot in a period of 51% to 60% of one walking cycle, the mean value of time series data of the vertical displacement of the toe of one foot in a period of 81% to 100% of one walking cycle, the mean value of time series data of the angle of one knee joint in a period of 11% to 20% of one walking cycle, the mean value of time series data of the angle of one knee joint in a period of 41% to 80% of one walking cycle, the mean value of time series data of the angle of one ankle joint in a period of 1% to 20% of one walking cycle, and the mean value of time series data of the angle of one ankle joint in a period of 51% to 80% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining sarcopenia by the prediction model created using the vertical displacement of the toe of one foot in the stance phase in isolation was 0.636, the AUC value obtained as a result of determining sarcopenia by the prediction model created using the vertical displacement of the toe of one foot in the swing phase in isolation was 0.514, the AUC value obtained as a result of determining sarcopenia by the prediction model created using the angle of the knee joint in the stance phase in isolation was 0.586, the AUC value obtained as a result of determining sarcopenia by the prediction model created using the angle of the knee joint in the swing phase in isolation was 0.699, the AUC value obtained as a result of determining sarcopenia by the prediction model created using the angle of the ankle joint in the stance phase in isolation was 0.498, and the AUC value obtained as a result of determining sarcopenia by the prediction model created using the angle of the ankle joint in the swing phase in isolation was 0.389.

On the other hand, the AUC value obtained as a result of determining sarcopenia by the prediction model created using the vertical displacement of the toe of one foot in the first period and the second period of the stance phase, the vertical displacement of the toe of one foot in the third period of the swing phase, the angle of the knee joint of one leg in the fourth period of the stance phase, the angle of the knee joint of one leg in the fifth period of the stance phase and the swing phase, the angle of the ankle joint of one foot in the sixth period of the stance phase, and the angle of the ankle joint of one foot in the seventh period of the stance phase and the swing phase was 0.881.

Accordingly, it is possible to determine sarcopenia more accurately in a prediction model created using a vertical displacement of the toe of one foot in the first period and the second period of the stance phase, a vertical displacement of the toe of one foot in the third period of the swing phase, an angle of the knee joint of one leg in the fourth period of the stance phase, an angle of the knee joint of one leg in the fifth period of the stance phase and the swing phase, an angle of the ankle joint of one foot in the sixth period of the stance phase, and an angle of the ankle joint of one foot in the seventh period of the stance phase and the swing phase than in a prediction model created using each of the vertical displacement of the toe of one foot in the stance phase, the vertical displacement of the toe of one foot in the swing phase, the angle of the knee joint of one leg in the stance phase, the angle of the knee joint of one leg in the swing phase, the angle of the ankle joint of one foot in the stance phase, and the angle of the ankle joint of one foot in the swing phase in isolation.

Subsequently, the walking parameters in the eighteenth modification of the present embodiment will be described.

The walking parameter in the eighteenth modification of the present embodiment may be a mean value of time series data of the vertical displacement of the toe of one foot in the first period of the stance phase of one leg, a mean value of time series data of the vertical displacement of the toe of one foot in the second period of the stance phase of one leg, a mean value of time series data of the angle of the knee joint of one leg in the third period of the stance phase of one leg, a mean value of time series data of the angle of the knee joint of one leg in the fourth period of the swing phase of one leg, a mean value of time series data of the angle of the ankle joint of one foot in the fifth period of the stance phase of one leg, a mean value of time series data of the angle of the ankle joint of one foot in the sixth period of the stance phase and the swing phase of one leg, and a mean value of time series data of the angle of the ankle joint of one foot in the seventh period of the swing phase of one leg.

In the eighteenth modification of the present embodiment, similar to the above experiment, time series data of the vertical displacement of the toe of one foot of each of the plurality of subjects, time series data of the angle of the knee joint of one leg of each of the plurality of subjects, and time series data of the angle of the ankle joint of one foot of each of the plurality of subjects were detected from the skeleton data of a plurality of subjects including a sarcopenia subject, a pre-sarcopenia subject, and a healthy subject. In addition, in the experiment, one normalized walking cycle was divided into ten intervals, and the mean value of the vertical displacements of the toe of one foot, the mean value of the angles of the knee joint of one leg, and the mean value of the angles of the ankle joint of one foot in one interval or two or more consecutive intervals were calculated for each subject.

Then, a prediction model was created with which of a sarcopenia subject, a pre-sarcopenia subject, and a healthy subject the subject is as an objective variable, and with the mean value of time series data of the vertical displacement of the toe of one foot in the first period of the stance phase, the mean value of time series data of the vertical displacement of the toe of one foot in the second period of the stance phase, the mean value of time series data of the angle of the knee joint of one leg in the third period of the stance phase, the mean value of time series data of the angle of the knee joint of one leg in the fourth period of the swing phase, the mean value of time series data of the angle of the ankle joint of one foot in the fifth period of the stance phase, the mean value of time series data of the angle of the ankle joint of one foot in the sixth period of the stance phase and the swing phase, and the mean value of time series data of the angle of the ankle joint of one foot in the seventh period of the swing phase as explanatory variables. The first period is a period of 11% to 20% of one walking cycle, the second period is a period of 41% to 60% of one walking cycle, the third period is a period of 41% to 50% of one walking cycle, the fourth period is a period of 61% to 100% of one walking cycle, the fifth period is a period of 11% to 20% of one walking cycle, the sixth period is a period of 31% to 70% of one walking cycle, and the seventh period is a period of 91% to 100% of one walking cycle. The prediction model was evaluated by cross validation. Leave-one-out cross validation was adopted as the cross validation. Then, the ROC curve of the prediction model in which a healthy subject and a pre-sarcopenia subject were determined was calculated. Furthermore, the AUC value of the ROC curve of the prediction model was calculated.

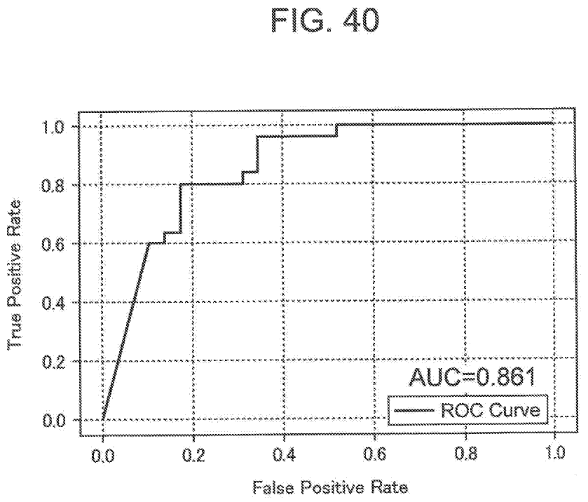

FIG. 40 is a view showing an ROC curve obtained as a result of determining a healthy subject and a pre-sarcopenia subject using the prediction model in the eighteenth modification of the present embodiment.

The prediction model in the eighteenth modification of the present embodiment was created with which of a sarcopenia subject, a pre-sarcopenia subject, and a healthy subject the subject is as an objective variable, and with each of the mean values of the vertical displacements of the toe of one foot in each of periods of 11% to 20% and 41% to 60% of one walking cycle, each of the mean values of the angles of one knee joint in each of periods of 41% to 50% and 61% to 100% of one walking cycle, and each of the mean values of the angles of one ankle joint in each of periods of 11% to 20%, 31% to 70%, and 91% to 100% of one walking cycle as explanatory variables. In FIG. 40, the vertical axis represents the true positive rate, and the horizontal axis represents the false positive rate. The true positive rate indicates a ratio at which the prediction model has correctly determined a pre-sarcopenia subject as a pre-sarcopenia subject, and the false positive rate indicates a ratio at which the prediction model has incorrectly determined a healthy subject as a pre-sarcopenia subject.

The ROC curve shown in FIG. 40 was obtained by plotting the true positive rate and the false positive rate of the prediction model created with the mean value of the vertical displacements of the toe of one foot in a period of 11% to 20% of one walking cycle, the mean value of the vertical displacements of the toe of one foot in a period of 41% to 60% of one walking cycle, the mean value of the angles of one knee joint in a period of 41% to 50% of one walking cycle, the mean value of the angles of one knee joint in a period of 61% to 100% of one walking cycle, the mean value of the angles of one ankle joint in a period of 11% to 20% of one walking cycle, the mean value of the angles of one ankle joint in a period of 31% to 70% of one walking cycle, and the mean value of the angles of one ankle joint in a period of 91% to 100% of one walking cycle as explanatory variables. The AUC value of the ROC curve shown in FIG. 40 was 0.861.

In the eighteenth modification of the present embodiment, the mean value of the vertical displacements of the toe of one foot in a period of 11% to 20% of one walking cycle, the mean value of the vertical displacements of the toe of one foot in a period of 41% to 60% of one walking cycle, the mean value of the angles of one knee joint in a period of 41% to 50% of one walking cycle, the mean value of the angles of one knee joint in a period of 61% to 100% of one walking cycle, the mean value of the angles of one ankle joint in a period of 11% to 20% of one walking cycle, the mean value of the angles of one ankle joint in a period of 31% to 70% of one walking cycle, and the mean value of the angles of one ankle joint in a period of 91% to 100% of one walking cycle are determined as walking parameters.

In addition, the prediction model created with the mean value of the vertical displacements of the toe of one foot in a period of 11% to 20% of one walking cycle, the mean value of the vertical displacements of the toe of one foot in a period of 41% to 60% of one walking cycle, the mean value of the angles of one knee joint in a period of 41% to 50% of one walking cycle, the mean value of the angles of one knee joint in a period of 61% to 100% of one walking cycle, the mean value of the angles of one ankle joint in a period of 11% to 20% of one walking cycle, the mean value of the angles of one ankle joint in a period of 31% to 70% of one walking cycle, and the mean value of the angles of one ankle joint in a period of 91% to 100% of one walking cycle as explanatory variables is determined as a prediction model to be used by the sarcopenia determination unit 113.

The walking parameter detection unit 112 detects time series data of the vertical displacement of the toe of one foot in the first period of the stance phase, time series data of the vertical displacement of the toe of one foot in the second period of the stance phase, time series data of the angle of the knee joint of one leg in the third period of the stance phase, time series data of the angle of the knee joint of one leg in the fourth period of the swing phase, time series data of the angle of the ankle joint of one foot in the fifth period of the stance phase, time series data of the angle of the ankle joint of one foot in the sixth period of the stance phase and the swing phase, and time series data of the angle of the ankle joint of one foot in the seventh period of the swing phase. The first period is a period of 11% to 20% of one walking cycle, the second period is a period of 41% to 60% of one walking cycle, the third period is a period of 41% to 50% of one walking cycle, the fourth period is a period of 61% to 100% of one walking cycle, the fifth period is a period of 11% to 20% of one walking cycle, the sixth period is a period of 31% to 70% of one walking cycle, and the seventh period is a period of 91% to 100% of one walking cycle.

The walking parameter detection unit 112 detects time series data of the vertical displacement of the toe of one foot in a period of 11% to 20% of one walking cycle, time series data of the vertical displacement of the toe of one foot in a period of 41% to 60% of one walking cycle, time series data of the angle of one knee joint in a period of 41% to 50% of one walking cycle, time series data of the angle of one knee joint in a period of 61% to 100% of one walking cycle, time series data of the angle of one ankle joint in a period of 11% to 20% of one walking cycle, time series data of the angle of one ankle joint in a period of 31% to 70% of one walking cycle, and time series data of the angle of one ankle joint in a period of 91% to 100% of one walking cycle.

In addition, the walking parameter detection unit 112 calculates the mean value of time series data of the vertical displacement of the toe of one foot in a period of 11% to 20% of one walking cycle, the mean value of time series data of the vertical displacement of the toe of one foot in a period of 41% to 60% of one walking cycle, the mean value of time series data of the angle of one knee joint in a period of 41% to 50% of one walking cycle, the mean value of time series data of the angle of one knee joint in a period of 61% to 100% of one walking cycle, the mean value of time series data of the angle of one ankle joint in a period of 11% to 20% of one walking cycle, the mean value of time series data of the angle of one ankle joint in a period of 31% to 70% of one walking cycle, and the mean value of time series data of the angle of one ankle joint in a period of 91% to 100% of one walking cycle.

The memory 12 stores in advance a prediction model generated with the mean value of time series data of the vertical displacement of the toe of one foot in the first period of the stance phase, the mean value of time series data of the vertical displacement of the toe of the one foot in the second period of the stance phase, the mean value of time series data of the angle of the knee joint of one leg in the third period of the stance phase, the mean value of time series data of the angle of the knee joint of the one leg in the fourth period of the swing phase, the mean value of time series data of the angle of the ankle joint of one foot in the fifth period of the stance phase, the mean value of time series data of the angle of the ankle joint of the one foot in the sixth period of the stance phase and the swing phase, and the mean value of time series data of the angle of the ankle joint of the one foot in the seventh period of the swing phase as input values and with which of a sarcopenia subject, a pre-sarcopenia subject, and a healthy subject the subject is as an output value.

The memory 12 stores in advance a prediction model generated with the mean value of time series data of the vertical displacement of the toe of one foot in a period of 11% to 20% of one walking cycle, the mean value of time series data of the vertical displacement of the toe of the one foot in a period of 41% to 60% of one walking cycle, the mean value of time series data of the angle of one knee joint in a period of 41% to 50% of one walking cycle, the mean value of time series data of the angle of the one knee joint in a period of 61% to 100% of one walking cycle, the mean value of time series data of the angle of one ankle joint in a period of 11% to 20% of one walking cycle, the mean value of time series data of the angle of the one ankle joint in a period of 31% to 70% of one walking cycle, and the mean value of time series data of the angle of the one ankle joint in a period of 91% to 100% of one walking cycle as input values and with which of a sarcopenia subject, a pre-sarcopenia subject, and a healthy subject the subject is as an output value.

The sarcopenia determination unit 113 determines whether or not the subject has sarcopenia using each of the mean values of time series data of the vertical displacement of the toe of one foot in each of the first period and the second period, each of the mean values of time series data of the angle of the knee joint of one leg in each of the third period and the fourth period, and each of the mean values of time series data of the angle of the ankle joint of one foot in each of the fifth period, the sixth period, and the seventh period.

The sarcopenia determination unit 113 determines which of a sarcopenia subject, a pre-sarcopenia subject, and a healthy subject the subject is by using the mean value of time series data of the vertical displacement of the toe of one foot in a period of 11% to 20% of one walking cycle, the mean value of time series data of the vertical displacement of the toe of one foot in a period of 41% to 60% of one walking cycle, the mean value of time series data of the angle of one knee joint in a period of 41% to 50% of one walking cycle, the mean value of time series data of the angle of one knee joint in a period of 61% to 100% of one walking cycle, the mean value of time series data of the angle of one ankle joint in a period of 11% to 20% of one walking cycle, the mean value of time series data of the angle of one ankle joint in a period of 31% to 70% of one walking cycle, and the mean value of time series data of the angle of one ankle joint in a period of 91% to 100% of one walking cycle.

By inputting the mean value of time series data of the vertical displacement of the toe of one foot in a period of 11% to 20% of one walking cycle, the mean value of time series data of the vertical displacement of the toe of one foot in a period of 41% to 60% of one walking cycle, the mean value of time series data of the angle of one knee joint in a period of 41% to 50% of one walking cycle, the mean value of time series data of the angle of one knee joint in a period of 61% to 100% of one walking cycle, the mean value of time series data of the angle of one ankle joint in a period of 11% to 20% of one walking cycle, the mean value of time series data of the angle of one ankle joint in a period of 31% to 70% of one walking cycle, and the mean value of time series data of the angle of one ankle joint in a period of 91% to 100% of one walking cycle to the prediction model, the sarcopenia determination unit 113 acquires, from the prediction model, a determination result indicating which of a sarcopenia subject, a pre-sarcopenia subject, or a healthy subject the subject is.

Thus, the AUC value obtained as a result of determining a pre-sarcopenia subject by the prediction model created using the vertical displacement of the toe of one foot in the stance phase in isolation was 0.560, the AUC value obtained as a result of determining a pre-sarcopenia subject by the prediction model created using the vertical displacement of the toe of one foot in the swing phase in isolation was 0.626, the AUC value obtained as a result of determining a pre-sarcopenia subject by the prediction model created using the angle of the knee joint of one leg in the stance phase in isolation was 0.537, the AUC value obtained as a result of determining a pre-sarcopenia subject by the prediction model created using the angle of the knee joint of one leg in the swing phase in isolation was 0.604, the AUC value obtained as a result of determining a pre-sarcopenia subject by the prediction model created using the angle of the ankle joint of one foot in the stance phase in isolation was 0.610, and the AUC value obtained as a result of determining a pre-sarcopenia subject by the prediction model created using the angle of the ankle joint of one foot in the swing phase in isolation was 0.622.

On the other hand, the AUC value obtained as a result of determining a pre-sarcopenia subject by the prediction model created using the vertical displacement of the toe of one foot in the first period and the second period of the stance phase, the angle of the knee joint of one leg in the third period of the stance phase, the angle of the knee joint of one leg in the fourth period of the swing phase, the angle of the ankle joint of one foot in the fifth period of the stance phase, the angle of the ankle joint of one foot in the sixth period of the stance phase and the swing phase, and the angle of the ankle joint of one foot in the seventh period of the swing phase was 0.861.

Accordingly, it is possible to determine a pre-sarcopenia subject more accurately in a prediction model created using a vertical displacement of the toe of one foot in the first period and the second period of the stance phase, an angle of the knee joint of one leg in the third period of the stance phase, an angle of the knee joint of one leg in the fourth period of the swing phase, an angle of the ankle joint of one foot in the fifth period of the stance phase, an angle of the ankle joint of one foot in the sixth period of the stance phase and the swing phase, and an angle of the ankle joint of one foot in the seventh period of the swing phase than in a prediction model created using each of the vertical displacement of the toe of one foot in the stance phase, the vertical displacement of the toe of one foot in the swing phase, the angle of the knee joint of one leg in the stance phase, the angle of the knee joint of one leg in the swing phase, the angle of the ankle joint of one foot in the stance phase, and the angle of the ankle joint of one foot in the swing phase in isolation.

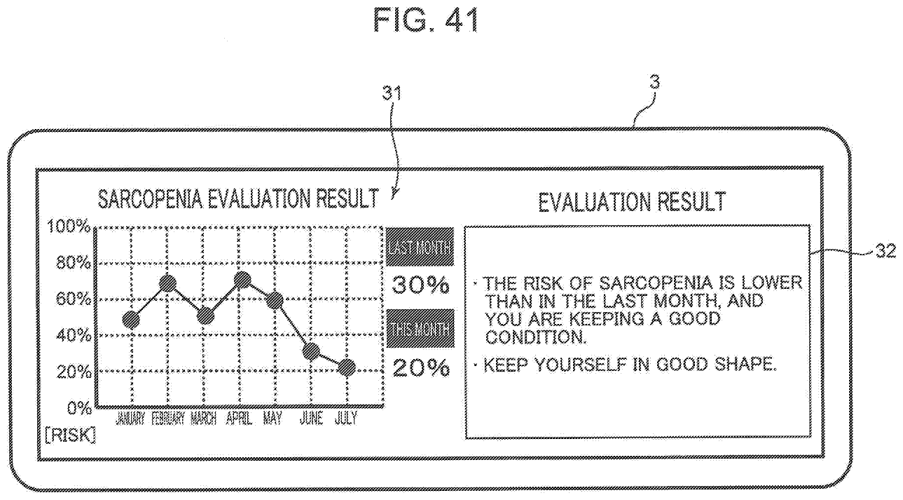

FIG. 41 is a view showing an example of an evaluation result screen displayed in the present embodiment.

The display unit 3 displays the evaluation result screen shown in FIG. 41. The evaluation result screen includes a sarcopenia evaluation presentation region 31 showing a past evaluation value of sarcopenia and a current evaluation value of sarcopenia, and an evaluation message 32. In the sarcopenia evaluation presentation region 31 of FIG. 41, evaluation of sarcopenia is performed once a month, and the evaluation values of sarcopenia for the past six months and the evaluation value of sarcopenia for this month are displayed.

The evaluation value of sarcopenia is a value indicating the possibility that the subject has sarcopenia, calculated by the prediction model. The value indicating the possibility that the subject has sarcopenia is represented by 0.0 to 2.0, for example. The evaluation result presentation unit 114 converts a value indicating the possibility that the subject has sarcopenia into a percentage and presents it as an evaluation value of sarcopenia.

In addition, in the second modification of the present embodiment, an average of the mean values of time series data of the angle of the knee joint of one leg of the sarcopenia subjects in a period of 50% to 60% of one walking cycle was 15.3 degrees, and an average of the mean values of time series data of the angle of the knee joint of one leg of the healthy subjects in a period of 50% to 60% of one walking cycle was 9.3 degrees. Therefore, the evaluation result presentation unit 114 may perform normalization such that 9.3 degrees becomes the minimum value of 0 and 15.3 degrees becomes the maximum value of 1, and convert, into a value between 0 and 1, the mean value of time series data of the angle of the knee joint of one leg of the subject in a period of 50% to 60% of one walking cycle calculated by the walking parameter detection unit 112. Then, the evaluation result presentation unit 114 may convert the converted value into a percentage and present it as an evaluation value of sarcopenia.

It is to be noted that in a case where the past evaluation value of sarcopenia is displayed together with the current evaluation value of sarcopenia, the sarcopenia determination unit 113 stores the evaluation value of the sarcopenia in the memory 12.

In addition, the sarcopenia evaluation presentation region 31 may display, as an evaluation result, whether or not the subject has sarcopenia. In addition, the sarcopenia evaluation presentation region 31 may display, as an evaluation result, which of a sarcopenia subject, a pre-sarcopenia subject, or a healthy subject the subject is.

In addition, the evaluation message 32 of “The risk of sarcopenia is lower than in the last month, and you are keeping a good condition. Keep yourself in good shape.” is displayed. When the evaluation value of sarcopenia of this month is lower than the evaluation value of sarcopenia of the last month and the evaluation value of sarcopenia of this month is lower than 0.5, the evaluation result presentation unit 114 reads the evaluation message 32 shown in FIG. 41 from the memory 12 and outputs it to the display unit 3.

It is to be noted that while in the present embodiment, the past evaluation values of sarcopenia are displayed together with the current evaluation value of sarcopenia, the present disclosure is not particularly limited to this, and the current evaluation value of sarcopenia may be displayed alone. In this case, the sarcopenia determination unit 113 is not required to store the evaluation value of sarcopenia in the memory 12.

In addition, the camera 2 in the present embodiment may be a security camera provided in front of the entrance, a camera slave machine of a video intercom, or a monitoring camera provided in a room. In addition, the display unit 3 may be a display of a smartphone, a tablet computer, or a video intercom.

It is to be noted that while in the present embodiment, the walking parameter detection unit 112 extracts skeleton data based on the moving image data acquired from the camera 2, the present disclosure is not particularly limited thereto, and skeleton data may be extracted using a motion capture system. The motion capture system may be optical, magnetic, mechanical, or inertial sensor based. For example, in an optical motion capture system, a camera captures an image of a subject with a marker attached to a joint and detects the position of the marker from the captured image. The walking parameter detection unit 112 acquires the skeleton data of the subject from the position data detected by the motion capture system. As the optical motion capture system, for example, a three-dimensional motion analysis device manufactured by Inter Reha Co., Ltd. is available.

In addition, the motion capture system may include a depth sensor and a color camera, and the motion capture system may automatically extract position information of a joint point of the subject from an image and detect the attitude of the subject. In this case, the subject does not need to attach the marker. As such a motion capture system, for example, Kinect manufactured by Microsoft Corporation is available.

In measurement of walking motion using a motion capture system, it is preferable that the angle of the ankle joint, the angle of the knee joint, or the vertical displacement of the toe in the walking motion is extracted from the position coordinates, and the feature amount of the walking motion is detected from the extracted angle or displacement.

It is to be noted that, in each of the above embodiments, each component may be configured by dedicated hardware or may be realized by executing a software program suitable for each component. Each component may be realized by a program execution unit such as a CPU or a processor reading and executing a software program recorded in a recording medium such as a hard disk or a semiconductor memory.

Some or all of the functions of the device according to the embodiment of the present disclosure are realized as a large scale integration (LSI), which is typically an integrated circuit. These may be individually integrated into one chip, or may be integrated into one chip so as to include some or all of them. In addition, the integrated circuit is not limited to LSI, and may be realized by a dedicated circuit or a general-purpose processor. A field programmable gate array (FPGA), which can be programmed after manufacturing the LSI, or a reconfigurable processor, which can reconfigure the connection and setting of the circuit cell inside the LSI, may be used.

In addition, some or all of the functions of the device according to the embodiment of the present disclosure may be realized by a processor such as a CPU executing a program.

In addition, all of the numerals used above are merely examples for specifically describing the present disclosure, and the present disclosure is not limited to the exemplified numerals.

In addition, the order of executing the steps shown in the flowchart is an example for the purpose of specifically describing the present disclosure, and may be any order other than the above as long as a similar effect is obtained. In addition, some of the above steps may be executed simultaneously (parallel) with other steps.

Since the technology according to the present disclosure can simply and highly accurately evaluate sarcopenia, it is useful for the technology of evaluating sarcopenia based on the walking motion of a subject.

This application is based on U.S. Provisional application No. 62/893,294 filed in United States Patent and Trademark Office on Aug. 29, 2019 and Japanese Patent application No. 2020-023431 filed in Japan Patent Office on Feb. 14, 2020, the contents of which are hereby incorporated by reference.

Although the present invention has been fully described by way of example with reference to the accompanying drawings, it is to be understood that various changes and modifications will be apparent to those skilled in the art. Therefore, unless otherwise such changes and modifications depart from the scope of the present invention hereinafter defined, they should be construed as being included therein.

1. A sarcopenia evaluation method in a sarcopenia evaluation device that evaluates sarcopenia based on a walking motion of a subject, the sarcopenia evaluation method comprising:
acquiring walking data related to walking of the subject;
detecting, from the walking data, at least one of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of an ankle joint of the one foot in the stance phase, and an angle of the ankle joint of the one foot in the swing phase; and
determining whether or not the subject has sarcopenia using at least one of the angle of the knee joint in the stance phase, the angle of the knee joint in the swing phase, the vertical displacement of the toe in the stance phase, the vertical displacement of the toe in the swing phase, the angle of the ankle joint in the stance phase, and the angle of the ankle joint in the swing phase.

2. The sarcopenia evaluation method according to claim 1, wherein
in the detection, time series data of an angle of the knee joint in a predetermined period of the swing phase is detected, and
in the determination, whether or not the subject has the sarcopenia is determined by using a mean value of the time series data of the angle of the knee joint.

3. The sarcopenia evaluation method according to claim 2, wherein
on a condition that a period from when one foot of the subject touches a ground to when the one foot touches the ground again is expressed as one walking cycle and the one walking cycle is expressed by 1% to 100%, the predetermined period is a period of 61% to 100% of the one walking cycle.

4. The sarcopenia evaluation method according to claim 1, wherein
in the detection, time series data of an angle of the knee joint in a predetermined period of the stance phase is detected, and
in the determination, whether or not the subject has the sarcopenia is determined by using a mean value of the time series data of the angle of the knee joint.

5. The sarcopenia evaluation method according to claim 4, wherein
on a condition that a period from when one foot of the subject touches a ground to when the one foot touches the ground again is expressed as one walking cycle and the one walking cycle is expressed by 1% to 100%, the predetermined period is a period of 50% to 60% of the one walking cycle.

6. The sarcopenia evaluation method according to claim 1, wherein
in the detection, time series data of the vertical displacement of the toe in a predetermined period of the stance phase is detected, and
in the determination, whether or not the subject has the sarcopenia is determined by using a mean value of the time series data of the vertical displacement of the toe.

7. The sarcopenia evaluation method according to claim 6, wherein
on a condition that a period from when one foot of the subject touches a ground to when the one foot touches the ground again is expressed as one walking cycle and the one walking cycle is expressed by 1% to 100%, the predetermined period is a period of 1% to 60% of the one walking cycle.

8. The sarcopenia evaluation method according to claim 1, wherein
in the detection, time series data of the vertical displacement of the toe in a predetermined period of the swing phase is detected, and
in the determination, whether or not the subject has the sarcopenia is determined by using a mean value of the time series data of the vertical displacement of the toe.

9. The sarcopenia evaluation method according to claim 8, wherein
on a condition that a period from when one foot of the subject touches a ground to when the one foot touches the ground again is expressed as one walking cycle and the one walking cycle is expressed by 1% to 100%, the predetermined period is a period of 65% to 70% of the one walking cycle.

10. The sarcopenia evaluation method according to claim 1, wherein
in the detection, time series data of a first angle of the ankle joint in a first period of the stance phase and time series data of a second angle of the ankle joint in a second period of the swing phase are detected, and
in the determination, whether or not the subject has the sarcopenia is determined by using a mean value of the time series data of the first angle of the ankle joint and a mean value of the time series data of the second angle of the ankle joint.

11. The sarcopenia evaluation method according to claim 1, wherein
in the detection, time series data of the vertical displacement of the toe in a first period of the stance phase, time series data of the angle of the knee joint in a second period of the stance phase, time series data of the angle of the knee joint in a third period of the swing phase, and time series data of the angle of the knee joint in a fourth period of the swing phase are detected, and
in the determination, whether or not the subject has the sarcopenia is determined by using a mean value of the time series data of the vertical displacement of the toe in the first period and each of mean values of the time series data of the angles of the knee joint in each of the second period, the third period, and the fourth period.

12. The sarcopenia evaluation method according to claim 1, wherein
in the detection, time series data of the vertical displacement of the toe in a first period of the stance phase, time series data of the angle of the ankle joint in a second period of the stance phase, time series data of the angle of the ankle joint in a third period of the stance phase, time series data of the angle of the ankle joint in a fourth period of the swing phase, and time series data of the angle of the ankle joint in a fifth period of the swing phase are detected, and
in the determination, whether or not the subject has the sarcopenia is determined by using a mean value of the time series data of the vertical displacement of the toe in the first period and each of mean values of the time series data of the angles of the ankle joint in each of the second period, the third period, the fourth period, and the fifth period.

13. The sarcopenia evaluation method according to claim 1, wherein
in the detection, time series data of the angle of the knee joint in a first period of the stance phase, time series data of the angle of the knee joint in a second period of the swing phase, time series data of the angle of the knee joint in a third period of the swing phase, time series data of the angle of the ankle joint in a fourth period of the stance phase, time series data of the angle of the ankle joint in a fifth period of the swing phase, and time series data of the angle of the ankle joint in a sixth period of the swing phase are detected, and
in the determination, whether or not the subject has the sarcopenia is determined by using each of mean values of the time series data of the angles of the knee joint in each of the first period, the second period, and the third period, and each of mean values of the time series data of the angles of the ankle joint in each of the fourth period, the fifth period, and the sixth period.

14. The sarcopenia evaluation method according to claim 1, wherein
in the detection, time series data of the vertical displacement of the toe in a first period of the stance phase, time series data of the vertical displacement of the toe in a second period of the stance phase, time series data of the vertical displacement of the toe in a third period of the swing phase, time series data of the angle of the knee joint in a fourth period of the stance phase, time series data of the angle of the knee joint in a fifth period of the stance phase and the swing phase, time series data of the angle of the ankle joint in a sixth period of the stance phase, and time series data of the angle of the ankle joint in a seventh period of the stance phase and the swing phase are detected, and
in the determination, whether or not the subject has the sarcopenia is determined by using each of mean values of the time series data of the vertical displacements of the toe in each of the first period, the second period, and the third period, each of mean values of the time series data of the angles of the knee joint in each of the fourth period and the fifth period, and each of mean values of the time series data of the angles of the ankle joint in each of the sixth period and the seventh period.

15. The sarcopenia evaluation method according to claim 1, further comprising:
determining whether or not the subject is a pre-sarcopenia subject, who will potentially have sarcopenia in future, using at least one of an angle of the knee joint in the stance phase, an angle of the knee joint in the swing phase, the vertical displacement of the toe in the stance phase, the vertical displacement of the toe in the swing phase, an angle of the ankle joint in the stance phase, and an angle of the ankle joint in the swing phase.

16. The sarcopenia evaluation method according to claim 1, wherein
in the determination, when an angle of the knee joint in the stance phase is larger than a threshold value, when an angle of the knee joint in the swing phase is larger than a threshold value, when the vertical displacement of the toe in the stance phase is larger than a threshold value, when the vertical displacement of the toe in the swing phase is larger than a threshold value, when an angle of the ankle joint in the stance phase is larger than a threshold value, or when an angle of the ankle joint in the swing phase is larger than a threshold value, it is determined that the subject has the sarcopenia.

17. The sarcopenia evaluation method according to claim 1, wherein
in the determination, whether or not the subject has the sarcopenia is determined by inputting at least one of an angle of the knee joint in the stance phase, an angle of the knee joint in the swing phase, the vertical displacement of the toe in the stance phase, the vertical displacement of the toe in the swing phase, an angle of the ankle joint in the stance phase, and an angle of the ankle joint in the swing phase that has been detected into a prediction model generated with at least one of an angle of the knee joint in the stance phase, an angle of the knee joint in the swing phase, the vertical displacement of the toe in the stance phase, the vertical displacement of the toe in the swing phase, an angle of the ankle joint in the stance phase, and an angle of the ankle joint in the swing phase as an input value, and with whether or not the subject has the sarcopenia as an output value.

18. A sarcopenia evaluation device that evaluates sarcopenia based on a walking motion of a subject, the sarcopenia evaluation device comprising:
an acquisition unit that acquires walking data related to walking of the subject;
a detection unit that detects, from the walking data, at least one of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of an ankle joint of the one foot in the stance phase, and an angle of the ankle joint of the one foot in the swing phase; and
a determination unit that determines whether or not the subject has sarcopenia using at least one of the angle of the knee joint in the stance phase, the angle of the knee joint in the swing phase, the vertical displacement of the toe in the stance phase, the vertical displacement of the toe in the swing phase, the angle of the ankle joint in the stance phase, and the angle of the ankle joint in the swing phase.

19. A non-transitory computer-readable recording medium in which a sarcopenia evaluation program that evaluates sarcopenia based on a walking motion of a subject is recorded, wherein
the non-transitory computer-readable recording medium causes a computer to function so as to acquire walking data related to walking of the subject,
so as to detect, from the walking data, at least one of an angle of a knee joint of one leg in a stance phase of the one leg of the subject, an angle of the knee joint of the one leg in a swing phase of the one leg, a vertical displacement of a toe of one foot in the stance phase, a vertical displacement of the toe of the one foot in the swing phase, an angle of an ankle joint of the one foot in the stance phase, and an angle of the ankle joint of the one foot in the swing phase, and
so as to determine whether or not the subject has sarcopenia using at least one of the angle of the knee joint in the stance phase, the angle of the knee joint in the swing phase, the vertical displacement of the toe in the stance phase, the vertical displacement of the toe in the swing phase, the angle of the ankle joint in the stance phase, and the angle of the ankle joint in the swing phase.
